(19) United States
(12) Patent Application Publication
(10) Pub. No.: US 2014/0242077 A1
(43) Pub. Date: Aug. 28, 2014

(54) METHODS AND COMPOSITIONS FOR MODULATING AN IMMUNE RESPONSE

(71) Applicant: AbbVie, Inc., North Chicago, IL (US)

(72) Inventors: Chee-Ho CHOI, Jefferson, MA (US); Qingfeng TAO, Newton, MA (US); Philip BARDWELL, Boston, MA (US); Tariq GHAYUR, Holliston, MA (US)

(73) Assignee: AbbVie, Inc., North Chicago, IL (US)

(21) Appl. No.: 14/162,716

(22) Filed: Jan. 23, 2014

Related U.S. Application Data
(60) Provisional application No. 61/755,835, filed on Jan. 23, 2013.

(51) Int. Cl. C07K 16/46; C07K 16/28; A61K 39/395

(52) U.S. Cl. CPC C07K 16/468; A61K 39/3955; C07K 16/2863; C07K 16/2896
USPC 424/136.1; 530/387.3; 536/23.53; 435/375; 435/320.1; 435/69.6; 435/7.24; 435/258.1; 435/254.11; 435/419; 435/328; 435/252.33; 435/254.21

(57) ABSTRACT

Provided are multispecific binding proteins and methods for using these multispecific binding proteins to modulate the activation state of immune cells, such as T-cells (e.g., cytotoxic T-cells). Also provided are methods of modulating an immune response (e.g., cell killing by cytotoxic T-cells) in a subject (e.g., a human subject). Also provided are nucleic acids, expression vectors and host cells encoding the multispecific binding proteins.

### RELATED APPLICATIONS

This application claims priority to U.S. Provisional Patent Applications 61/755,835, filed Jan. 23, 2013, which is incorporated herein by reference in its entirety.

### BACKGROUND

Immunotherapy is the treatment of a disease or condition through the induction, enhancement, or suppression of an immune response in a subject. The use of immunotherapy (also called biologic therapy or biotherapy) for specific treatment of diseases and conditions is an avenue that has been pursued for many years with variable amounts of success.

Immunotherapeutics (also known as immunomodulators) are now used to treat a wide variety of disorders including, for example, Alzheimer disease (AD), immune disorders, and various cancers (including leukemia, lymphoma, and multiple myeloma). Certain kinds of immunotherapeutics function through modulation of effector cell activation and recruitment (e.g. cytotoxic T-cells). This type of effector cell modulation may include, for example, “redirected cytotoxicity” (rCTL) in order to destroy target cells. However, modulation of the immune response in order to engineer these types of effects has proven difficult to control.

Subjects treated with current immunotherapeutics can suffer side effects arising from modulation of the immune system. These side effects (also called “immunotoxicities”) are reactions against normal tissues and can range from relatively minor conditions to serious toxicities involving major organs such as the lung and liver. At their most severe, these immunotoxic effects can result in cytokine release syndrome (CRS), also called “cytokine storm,” in which release of proinflammatory cytokines result in multi-organ failure and death. Further complicating matters, certain immunotherapeutics have proven to be ineffective, possibly because they only target one molecule or cell type in a complex condition requiring a more robust approach.

Accordingly, there is a need in the art for novel compositions and methods that allow for the efficient and effective modulation of an immune response in order to treat a disease or condition. In particular, these compositions and methods should have reduced immunotoxicities, thereby mitigating the potentially lethal effects of cytokine release in the subject being treated.

### SUMMARY

The present disclosure provides multispecific binding proteins and methods for using these multispecific binding proteins to modulate the activation state of immune cells, such as T-cells (e.g., cytotoxic T-cells). Also provided are methods of modulating an immune response (e.g., cell killing by effector cells, such as cytotoxic T-cells) in a subject (e.g., a human subject). Also provided are nucleic acids, expression vectors and host cells encoding the multispecific binding proteins. The methods and compositions disclosed herein are particularly useful for treating immune disorders or cancer in that they allow for precise control of the immunological effect of an immunomodulatory therapeutic. For example, the multispecific binding proteins and methods disclosed herein allow for directed lysis of a target cell (e.g., a tumor cell) in a subject by redirecting CTLs (cytotoxic T lymphocytes) to the target cell, whilst at the same time reducing the cytokine burst of the redirected T-cells, thereby reducing the side effects of the treatment. Additionally or alternatively, the multispecific binding proteins and methods disclosed herein can also be used to reduce inflammation in a subject by, e.g., reducing the activation state of immune cells such as T-cells (e.g., cytotoxic T-cells).

The methods and compositions disclosed herein are also particularly advantageous for the elucidation of one or more inter and intra immune/cellular targets that are required for modulating an immune response. Specifically, these methods and compositions provide: 1) a discovery engine for elucidating intra and inter immune/cellular targets for modulating an immune response; 2) immunotherapeutics (for example, DVD-Ig, or related molecules) for modulating an immune response; 3) methods for treating a disorder or disease, including, e.g., an immune response, a cancer, a neurological condition, a pain condition, a pathogenic condition, or a congenital condition.

Accordingly, in one aspect the present disclosure provides a multispecific binding protein comprising a multispecific binding protein comprising a first binding site that specifically binds to a target cell antigen, a second binding site that specifically binds to a cell surface receptor on an immune cell, and a third binding site that specifically binds to cell surface modulator on the immune cell.

In certain embodiments, the immune cell is a myeloid cell or a lymphoid cell. In certain embodiments, the immune cell is an effector cell. In certain embodiments, the immune cell is a T-cell, macrophage, dendritic cell, natural killer cell or eosinophil. In certain embodiments, the immune cell is a cytotoxic T-cell. In certain embodiments, the cell surface receptor on the immune cell is a T-cell receptor (TCR) complex component.

In certain embodiments, the multispecific binding protein comprises a first binding site that specifically binds to a target cell antigen, a second binding site that specifically binds to a T-cell receptor (TCR) complex component on a T-cell, and a third binding site that specifically binds to a T-cell modulator on the T-cell.

In certain embodiments, the target cell antigen is a disease-associated antigen. In certain embodiments, the target cell antigen is tumor-associated antigen. In certain embodiments, the target cell antigen is CD3, CD19, CD20, CD80; CD22, CD30, CD40, EGFR, HER2, HER3, HER4, IGF1, IGF12, IGF1R, RON, HGF, c-MET, VEGF, DLL4, NRP1, PLGF, EpCAM, CEA, PSMA, or TRAIL-R.

In certain embodiments, the TCR complex component is CD3γ, CD3δ, or CD3ε. In certain embodiments, the TCR complex component is CD3ε.

In certain embodiments, the modulator is a stimulator of T-cell activation. In certain embodiments, the modulator is an inhibitor of T-cell activation. In certain embodiments, the modulator is CD2, 4-1BB, PD1, LAG3, CTLA4, GITR CD80, CD86, PD-L1, PD-L2, B7-H1, B7-H3, B7-H4, HVEM, ILT3, ILT4, BTLA, CD160, MHC-1, CD40, ICOSL, CD70, OX40L, 4-1BBL, GITRL, LIGHT, TIM3, TIM4, ICAM1, LFA3, CD28, CD40L, ICOS, CD27, OX40, Galectin 9, TIM1, DR3, CD30, SLAM, 2B4, TIGIT, CD226, CD160, LAIR1, CD96, CRTAM, CD226, CD352, CD319, BTN1, BTN2, BTN3, CD120a, CD120b, TNFRSF3, TNFRSF6B, TNFRSF10A, TNFRSF10B, TNFRSF10C, TNFRSF10D, TNFRSF19, TNFRSF19L, TNFRSF25, TNFRSF27, EDAR, LFA1, CD95, CD265, CD267, CD268, CD269, CD358, or CD271.

In certain embodiments, the target cell antigen is EGFR, the TCR complex component is CD3ε, and T-cell modulator is CD2. In certain embodiments, the target cell antigen is EGFR, the TCR complex component is CD3ε, and T-cell modulator is 41BB.

In certain embodiments, the multispecific binding protein is a DVD-Ig, half-DVD-Ig, a scDVD-Ig, an fDVD-Ig, an rDVD-Ig, a pDVD-Ig, an mDVD-Ig or a coDVD-Ig. In certain embodiments, the multispecific is a pDVD-Ig.

In certain embodiments, the pDVD-Ig comprises first, second, third and fourth polypeptide chains, wherein said first polypeptide chain comprises VD1-(X1)n-VD2-C-(X2)n, wherein VD1 is a first heavy chain variable domain, VD2 is a second heavy chain variable domain, C is a constant domain, X1 is a linker with the proviso that it is not a constant domain, and X2 is an Fc region; wherein said second polypeptide chain comprises VD3-(X1)n-VD4-C-(X2)n, wherein VD3 is a first light chain variable domain, VD4 is a second light chain variable domain, C is a constant domain, X1 is a linker with the proviso that it is not a constant domain, and X2 does not comprise an Fc region; wherein said third polypeptide chain comprises VD5-C-(X3)n, wherein VD5 is a third heavy chain variable domain, C is a constant domain, and X3 is an Fc region; wherein said fourth polypeptide chain comprises VD6-C-(X3)n, wherein VD6 is a third light chain variable domain, C is a constant domain, and X4 does not comprise an Fc region; wherein n is 0 or 1, and wherein the VD1 and VD3 domains on the first and second polypeptide chains form one functional binding site for a first antigen, the VD2 and VD4 domains on the first and second polypeptide chains form one functional binding site for a second antigen, and the VD5 and VD6 domains on the third and fourth polypeptide chains form one functional binding site for a third antigen. In certain embodiments of the pDVD-Ig, the Fc region of the first and third polypeptide chains each comprises a mutation, wherein said mutations on the two Fc regions enhance heterodimerization of the first and third polypeptide chains.

In certain embodiments, the first antigen is the target cell antigen. In certain embodiments, the second antigen is the TCR complex component. In certain embodiments, the third antigen is the T-cell modulator.

In certain embodiments of the pDVD-Ig, VD1, VD2, or VD5 comprises a CDR region amino acid sequence selected from SEQ ID NO: 88-111. In certain embodiments, the pDVD-Ig multispecific binding protein comprises: (a) VD1, VD2, or VD5 comprises HCDR1-3 regions selected from the group consisting of SEQ ID NO: 88, 89, and 90; 94, 95, and 96; 100, 101, and 102; and 106, 107, and 108; (b) VD1, VD2, and VD5 comprise HCDR1-3 regions selected from the group consisting of SEQ ID NO: 88, 89, and 90; 94, 95, and 96; 100, 101, and 102; and 106, 107, and 108; (c) VD1, VD2, or VD5 comprise a VH amino acid sequence selected from the group consisting of SEQ ID NO: 80, 82, 84, and 86; (d) VD2 and VD5 comprise SEQ ID NO: 86 and 80, respectively; (e) VD2 and VD5 comprise SEQ ID NO: 86 and 82, respectively; (f) VD1, VD2 and VD5 comprise SEQ ID NO: 84, 86 and 80, respectively; or (g) VD1, VD2 and VD5 comprise SEQ ID NO: 84, 86 and 82, respectively.

In certain embodiments, the pDVD-Ig multispecific binding protein comprises: (a) VD3, VD4, or VD6 comprise LCDR1-3 regions selected from the group consisting of SEQ ID NO: 91, 92, and 93; 97, 98, and 99; 103, 104, and 105; and 109, 110, and 111; (b) VD3, VD4, and VD6 comprise LCDR1-3 regions selected from the group consisting of SEQ ID NO: 91, 92, and 93; 97, 98, and 99; 103, 104, and 105; and 109, 110, and 111; (c) VD3, VD4, or VD6 comprise a VL amino acid sequence selected from the group consisting of SEQ ID NO: 81, 83, 85, and 87; (d) VD3 and VD6 comprise SEQ ID NO: 81 and 87, respectively; or (e) VD3 and VD6 comprise SEQ ID NO: 83 and 87, respectively; (f) VD3, VD4, and VD6 comprise SEQ ID NO: 84, 87 and 81, respectively; or (g) VD3, VD4, and VD6 comprise SEQ ID NO: 84, 87 and 83, respectively.

In certain embodiments, the pDVD-Ig multispecific binding protein comprises a polypeptide sequence selected from the group consisting of SEQ ID NO: 32-79.

In certain embodiments, the pDVD-Ig multispecific binding protein comprises first, second, third and fourth polypeptide chains comprise the amino acid sequences set forth in SEQ ID NO: 32, 33, 34, and 35; 36, 37, 38, and 39; 40, 41, 42, and 43; 44, 45, 46, and 47; 48, 49, 50, and 51; 52, 53, 54, and 55; 56, 57, 58, and 59; 60, 61, 62, and 63; 64, 65, 66, and 67; 68, 69, 70, and 71; 72, 73, 74, and 75; 76, 77, 78, and 79, respectively.

In another aspect, the present disclosure provides a therapeutic combination comprising: a first multispecific binding protein comprising a first binding site that specifically binds to a target cell antigen, and a second binding site that specifically binds to a T-cell receptor (TCR) complex component on a T-cell; and a second multispecific binding protein comprising comprising a first binding site that specifically binds to a target cell antigen, and a second binding site that specifically binds to a T-cell modulator on the T-cell.

In certain embodiments, the target cell antigen is a disease-associated antigen. In certain embodiments, the target cell antigen is tumor-associated antigen. In certain embodiments, the TCR complex component is CD3γ, CD3δ, or CD3ε. In certain embodiments, the TCR complex component is CD3ε. In certain embodiments, the T-cell modulator is a stimulator of T-cell activation. In certain embodiments, the T-cell modulator is an inhibitor of T-cell activation. In certain embodiments, the cell surface modulator is CD2, 4-1BB, PD1, LAG3, CTLA4, GITR CD80, CD86, PD-L1, PD-L2, B7-H3, B7-H4, HVEM, ILT3, ILT4, BTLA, CD160, MHC-1, CD40ICOSL, CD70, OX40L, 4-1BBL, GITRL, LIGHT, TIM3, TIM4, ICAM1, LFA3, CD28, CD40L, ICOS, CD27, OX40, Galectin 9, TIM1, or LFA1. In certain embodiments, the target cell antigen is EGFR, the TCR complex component is CD3ε, and T-cell modulator is CD2. In certain embodiments, the target cell antigen is EGFR, the TCR complex component is CD3ε, and T-cell modulator is 41BB.

In certain embodiments, the first and/or second multispecific binding protein is a DVD-Ig, half-DVD-Ig, a scDVD-Ig, an fDVD-Ig, an rDVD-Ig, a pDVD-Ig, an mDVD-Ig or a coDVD-Ig.

In certain embodiments of the therapeutic combination, the first multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 100, 101, 102; 106, 107, and 108; (b) VD1 or VD2 comprises HCDR1-3 regions selected from the group consisting of SEQ ID NO: 100, 101, 102; 106, 107, and 108; (c) VD1 and VD2 comprise HCDR1-3 regions selected from the group consisting of SEQ ID NO: 100, 101, 102; 106, 107, and 108; (d) VD1 or VD2 comprise a VH amino acid sequence selected from the group consisting of SEQ ID NO: 84 and 86; (e) VD1 and VD2 comprise SEQ ID NO: 84 and 86, respectively.

In certain embodiments of the therapeutic combination, the first multispecific binding protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 103, 104, 105, 109, 110, and 111; (b) VD1 or VD2 comprises LCDR1-3 regions selected from the group consisting of SEQ ID NO: 103, 104, and 105; and 109, 110, and 111; (c) VD1 and VD2 comprise LCDR1-3 regions selected from the group consisting of SEQ ID NO: 103, 104, and 105; and 109, 110, and 111; (d) VD1 or VD2 comprise a VL amino acid sequence selected from the group consisting of SEQ ID NO: 85, and 87; (e) VD1 and VD2 comprise SEQ ID NO: 85 and 87, respectively.

In certain embodiments of the therapeutic combination, the second multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 88, 89, 90; 94, 95, 96; 100, 101, and 102; (b) VD1 or VD2 comprises HCDR1-3 regions selected from the group consisting of SEQ ID NO: 88, 89, and 90; 94, 95, and 96; and 100, 101, and 102; (c) VD1 and VD2 comprise HCDR1-3 regions selected from the group consisting of SEQ ID NO: 88, 89, and 90; 94, 95, and 96; and 100, 101, and 102; (d) VD1 or VD2 comprise a VH amino acid sequence selected from the group consisting of SEQ ID NO: 80, 82, and 84; (e) VD1 and VD2 comprise SEQ ID NO: 80 and 84, respectively; or (f) VD2 and VD2 comprise SEQ ID NO: 82 and 84, respectively.

In certain embodiments of the therapeutic combination, the second multispecific binding protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 91, 92, 93; 97, 98, 99; 103, 104, and 105; (b) VD1 or VD2 comprises LCDR1-3 regions selected from the group consisting of SEQ ID NO: 91, 92, and 93; 97, 98, and 99; and 103, 104, and 105; (c) VD1 and VD2 comprise LCDR1-3 regions selected from the group consisting of SEQ ID NO: 91, 92, and 93; 97, 98, and 99; and 103, 104, and 105; (d) VD1 or VD2 comprise a VL amino acid sequence selected from the group consisting of SEQ ID NO: 81, 83, and 87; (e) VD1 and VD2 comprise SEQ ID NO: 81 and 85, respectively; or (f) VD2 and VD2 comprise SEQ ID NO: 83 and 85, respectively.

In another aspect, the present disclosure provides a method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein.

In another aspect, the instant disclosure provides a method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein, wherein the multispecific binding protein induces less cytokine production in the subject compared to administration of a equivalent effective amount of a therapeutic combination as disclosed herein.

In another aspect, the instant disclosure provides method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein, wherein the multispecific binding protein induces greater T-cell activation in the subject compared to administration of an equivalent effective amount of a therapeutic combination as disclosed herein.

In certain embodiments, the subject is a mammalian subject, e.g., mice, rats, gerbils, hamsters, rabbits, apes, monkeys, humans, dogs, cats, camels, llamas, cattle and horses. In certain embodiments, the subject is a human subject.

In another aspect, the instant disclosure provides a method of modulating the activation state of a T-cell, the method comprising contacting the T-cell with a multispecific comprising: a first binding site that specifically binds to a first T-cell modulator; and a second binding site that specifically binds to a second T-cell modulator, as disclosed herein. In certain embodiments, the first and second T-cell modulator is a stimulator of T-cell activation, wherein the multispecific binding protein inhibits T-cell activation. In certain embodiments, the first and second T-cell modulator is an inhibitor of T-cell activation, wherein the T-cell exhibits activation.

In another aspect, the instant disclosure provides a multispecific binding protein comprising: a first binding site that specifically binds to a first cell surface modulator; and a second binding site that specifically binds to a second cell surface modulator.

In certain embodiments, the first or second modulator is a stimulator of T-cell activation. In certain embodiments, the first and second modulator is a stimulator of T-cell activation. In certain embodiments, the first or second modulator is an inhibitor of T-cell activation. In certain embodiments, the first and second modulator is an inhibitor of T-cell activation.

In certain embodiments, the first or second cell surface modulator is CD2, 4-1BB, PD1, LAG3, CTLA4, GITR CD80, CD86, PD-L1, PD-L2, B7-H1, B7-H3, B7-H4, HVEM, ILT3, ILT4, BTLA, CD160, MHC-1, CD40, ICOSL, CD70, OX40L, 4-1BBL, GITRL, LIGHT, TIM3, TIM4, ICAM1, LFA3, CD28, CD40L, ICOS, CD27, OX40, Galectin 9, TIM1, DR3, CD30, SLAM, 2B4, TIGIT, CD226, CD160, LAIR1, CD96, CRTAM, CD226, CD352, CD319, BTN1, BTN2, BTN3, CD120a, CD120b, TNFRSF3, TNFRSF6B, TNFRSF10A, TNFRSF10B, TNFRSF10C, TNFRSF10D, TNFRSF19, TNFRSF19L, TNFRSF25, TNFRSF27, EDAR, LFA1, CD95, CD265, CD267, CD268, CD269, CD358, or CD271.

In certain embodiments, the first T-cell modulator is 41BB and the second T-cell modulator is LAG3. In certain embodiments, the first T-cell modulator is PD1 and the second T-cell modulator is LAG3. In certain embodiments, the first T-cell modulator is CTLA4 and the second T-cell modulator is LAG3. In certain embodiments, the first T-cell modulator is GITR and the second T-cell modulator is LAG3. In certain embodiments, the first T-cell modulator is PD1 and the second T-cell modulator is PD1, wherein the first and second binding site binds to different epitopes of PD1. In certain embodiments, the first T-cell modulator is PD1 and the second T-cell modulator is CTLA4.

In certain embodiments, the multispecific binding protein is a DVD-Ig, half-DVD-Ig, a scDVD-Ig, an fDVD-Ig, an rDVD-Ig, a pDVD-Ig, an mDVD-Ig or a coDVD-Ig. In certain embodiments, the multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 120, 121, 122, 126, 127, 128, 132, 134, 134, 138, 139, 140; (b) VD1 or VD2 comprises HCDR1-3 regions selected from the group consisting of SEQ ID NO: 120, 121, and 122; 126, 127, and 128; 132, 134, and 134, and 138, 139, and 140; (c) VD1 and VD2 comprise HCDR1-3 regions selected from the group consisting of SEQ ID NO: 120, 121, and 122; 126, 127, and 128; 132, 134, and 134, and 138, 139, and 140; (d) VD1 or VD2 comprise a VH amino acid sequence selected from the group consisting of SEQ ID NO: 112, 114, 115, 116, 118; (e) VD1 and VD2 comprise a VH amino acid sequence selected from the group consisting of SEQ ID NO: 112, 114, 115, 116, 118; or (f) the polypeptide chain comprises an amino acid sequence selected from the group consisting of SEQ ID NO: 144, 146, 148, 150, 152, 154, 156, and 158.

In certain embodiments, the multispecific binding protein comprises a polypeptide chain a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 123, 124, 125, 129, 130, 131, 135, 136, 137, 141, 142, 143; (b) VD1 or VD2 comprises HCDR1-3 regions selected from the group consisting of SEQ ID NO: 123, 124, and 125; 129, 130, and 131; 135, 136, and 137; and 141, 142, and 143; (c) VD1 and VD2 comprise HCDR1-3 regions selected from the group consisting of SEQ ID NO: 123, 124, and 125; 129, 130, and 131; 135, 136, and 137; and 141, 142, and 143; (d) VD1 or VD2 comprise a VL amino acid sequence selected from the group consisting of SEQ ID NO: 113, 115, 117, and 119; (e) VD1 and VD2 comprise a VL amino acid sequence selected from the group consisting of SEQ ID NO: 113, 115, 117, and 119; or (f) the polypeptide chain comprises an amino acid sequence selected from the group consisting of SEQ ID NO: 145, 147, 149, 151, 153, 155, 157, and 159.

In another aspect, the instant disclosure provides a method of modulating the activation state of a T-cell, the method comprising contacting the T-cell with a multispecific binding protein comprising a first binding site that specifically binds to a first T-cell modulator and a second binding site that specifically binds to a second T-cell modulator, as disclosed herein.

In certain embodiments, the first and second T-cell modulator is a stimulator of T-cell activation, and wherein the multispecific binding protein inhibits T-cell activation. In certain embodiments, the first and second T-cell modulator is an inhibitor of T-cell activation, and wherein the T-cell is activated.

In another aspect, the instant disclosure provides a method of directing a cytotoxic T-cell to lyse a target cell in a subject, the method comprising administering to the subject an effective amount of a multispecific binding protein comprising a first binding site that specifically binds to a first T-cell modulator and a second binding site that specifically binds to a second T-cell modulator, as disclosed herein. In certain embodiments, the target cell is a tumor cell.

In another aspect, the instant disclosure provides a method of reducing inflammation in a subject, the method comprising administering to the subject an effective amount of a multispecific binding protein comprising a first binding site that specifically binds to a first T-cell modulator and a second binding site that specifically binds to a second T-cell modulator, as disclosed herein. In certain embodiments, the activation state of T-cells in the subject are reduced.

In another aspect, the instant disclosure provides a multispecific binding protein as disclosed herein, wherein the binding protein is a crystallized binding protein.

In another aspect, the instant disclosure provides an isolated nucleic acid encoding the binding protein amino acid sequence of any of the preceding claims. In another aspect, the instant disclosure provides a vector comprising the isolated nucleic acid of claim. In another aspect, the instant disclosure provides host cell comprising the nucleic acid or vector. In another aspect, the instant disclosure provides a method of producing a multispecific binding protein, comprising culturing the host cell described of claim 67 in culture medium under conditions sufficient to produce the binding protein.

In another aspect, the instant disclosure provides pharmaceutical composition comprising a multispecific binding protein disclosed herein, and a pharmaceutically acceptable carrier.

In another aspect, the invention provides a method of identifying a multispecific binding protein that modulates a T-cell response, the method comprising: contacting a population of cytotoxic T-cells with a population of target cells in the presence and absence of a multispecific binding protein, wherein the multispecific binding protein simultaneously and specifically binds to a cell surface co-stimulator or co-repressor on T-cells and to a cell surface antigen on the target cells; and measuring the amount cell killing of the population of target cells, wherein an increase or decrease in cell killing in the presence of the multispecific binding, compared to the absence of the multispecific binding protein, identifies the multispecific binding protein as modulator of a T-cell response.

In another aspect, the invention provides a method of identifying a multispecific binding protein that modulates a T-cell response, the method comprising: contacting a population of cytotoxic T-cells with a population of antigen presenting cells in the presence and absence of a multispecific binding protein, wherein the multispecific binding protein simultaneously and specifically binds to two cell surface co-stimulators or co-repressors on T-cells; and measuring the amount of cytokine released from the population of T-cells, wherein an increase or decrease in amount of cytokine released in the presence of the multispecific binding, compared to the absence of the multispecific binding protein, identifies the multispecific binding protein as modulator of a T-cell response.

In another aspect, the invention provides a method of modulating an immune response in a subject, the method comprising: administering a therapeutic amount of a multispecific binding protein that simultaneously and specifically binds to a cell surface co-stimulator or co-repressor on an immune cell (e.g., a T-cell) and to a cell surface antigen on a target cell, wherein the multispecific binding protein modulates a normal response of the immune cell (e.g., T-cell) to target cell binding.

### BRIEF DESCRIPTION OF THE DRAWINGS

FIG. 1 depicts the results of experiments investigating the effect of T-cell co-stimulator X engagement on DVD-Ig induced tumor cell killing by human T-cells.

FIG. 2 depicts the results of experiments investigating the effect of T-cell co-stimulator Y engagement on DVD-Ig induced tumor cell killing by human T-cells.

FIG. 3 depicts the results of experiments investigating the effect of T-cell co-stimulator CD2 engagement upon human T-cell activity.

FIG. 4 depicts the results of experiments investigating the effect of T-cell co-stimulator 4-1BB engagement upon human T-cell activity.

FIG. 5 depicts a design schematic of tri-specific, monovalent poly-Ig proteins and their respective size exclusion chromatography (SEC) profiles.

FIG. 6 depicts the results of experiments measuring the affinity of Poly-Ig proteins for targets/target cells.

FIG. 7 depicts the results of experiments measuring the affinity of Poly-Ig protein binding domains for targets.

FIG. 8 depicts the results of experiments measuring the affinity of Poly-Ig protein binding domains for targets, and the SEC profiles of the Poly-Ig proteins.

FIG. 9 depicts the results of experiments measuring the affinity of Poly-Ig protein binding domains for targets, and the SEC profiles of the Poly-Ig proteins.

FIG. 10 depicts the results of experiments investigating the effect of Poly-Ig proteins comprising T-cell regulatory binding domains upon human T-cell activity.

FIG. 11 depicts the results of experiments investigating the effect of Poly-Ig proteins comprising T-cell regulatory binding domains upon human T-cell activity.

FIG. 12 depicts the results of experiments using FACS to determine the functional binding affinity of antibodies or DVD-Igs to co-stimulatory molecules on activated human T-cells.

FIG. 13 depicts the results of experiments investigating the effect of T-cell co-stimulator B and/or D engagement upon the release of cytokines by human T-cells.

FIG. 14 depicts the results of experiments investigating the effect of T-cell co-stimulator engagement upon the release of cytokines by human T-cells.

FIG. 15 depicts the results of experiments investigating the effect of T-cell co-stimulator engagement upon the proliferation of human T-cells.

FIG. 16 depicts the results of experiments investigating the effect of T-cell co-stimulator engagement upon the secretion/release of cytokines by human T-cells.

FIG. 17 depicts the results of experiments investigating the effect of T-cell co-stimulator engagement upon the secretion of cytokines by human T-cells.

### DETAILED DESCRIPTION

Provided are multispecific binding proteins and methods for using these multispecific binding proteins to modulate the activation state of immune cells, such as T-cells (e.g., cytotoxic T-cells). The invention also provides methods of modulating an immune response (e.g., cell killing by cytotoxic T-cells) in a subject (e.g., a human subject). Also provided are nucleic acids, expression vectors and host cells encoding the multispecific binding proteins.

### I. DEFINITIONS

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, genetics and protein and nucleic acid chemistry and hybridization described herein are those well known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The term “multispecific binding protein” is used throughout this specification to denote a binding protein comprising two or more antigen binding sites, each of which can bind independently bind to an antigen.

The terms “dual variable domain binding protein” and “dual variable domain immunoglobulin” refer to a binding protein that has two variable domains in each polypeptide chain of its binding arm(s) (e.g., a pair of HC/LC) (see PCT Publication No. WO 02/02773), each of which is able to bind to an antigen. In an embodiment, each variable domain binds different antigens or epitopes. In another embodiment, each variable domain binds the same antigen or epitope. In another embodiment, a dual variable domain binding protein has two identical antigen binding arms, with identical specificity and identical CDR sequences, and is bivalent for each antigen to which it binds. In an embodiment, the DVD binding proteins may be monospecific, i.e., capable of binding one antigen or multispecific, i.e., capable of binding two or more antigens. DVD binding proteins comprising two heavy chain DVD polypeptides and two light chain DVD polypeptides are referred to as a DVD-Ig™.

The terms “single chain dual variable domain immunoglobulin” or “scDVD-Ig™” or scFvDVDIg™ “refer to the antigen binding fragment of a DVD molecule that is analogous to an antibody single chain Fv fragment. scDVD-Ig™ are described in U.S. Ser. No. 61/746,659, incorporated herein by reference in its entirety. scDVD-Ig™ are generally of the formula VH1-(X1)n-VH2-X2-VL1-(X3)n-VL2, where VH1 is a first antibody heavy chain variable domain, X1 is a linker with the proviso that it is not a constant domain, VH2 is a second antibody heavy chain variable domain, X2 is a linker, VL1 is a first antibody light chain variable domain, X3 is a linker with the proviso that it is not a constant domain, VL2 is a second antibody light chain variable domain, and n is 0 or 1, where the VH1 and VL1, and the VH2 and VL2 respectively combine to form two functional antigen binding sites.

The terms “DVD-Fab” or fDVD-Ig™” refer to the antigen binding fragment of a DVD-Ig™ molecule that is analogous to an antibody Fab fragment. fDVD-Ig™ are described in U.S. Ser. No. 61/746,663, incorporated herein by reference in its entirety. In certain embodiments, fDVD-Ig™ include a first polypeptide chain having the general formula VH1-(X1)n-VH2-C-(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library. In certain embodiments, the fDVD-Ig™ also include a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites. In certain embodiments, the first and second polypeptide chains combine to form a fDVD-Ig™.

The terms “receptor DVD-Ig™” constructs, or “rDVD-Ig™” refer to DVD-Ig™ constructs comprising at least one receptor-like binding domain. rDVD-Ig™ are described in U.S. Ser. No. 61/746,616, incorporated herein by reference in its entirety. Variable domains of the rDVD-Ig™ molecule may include one immunoglobulin variable domain and one non-immunoglobulin variable domain such as a ligand binding domain of a receptor, or an active domain of an enzyme. rDVD-Ig™ molecules may also comprise two or more non-Ig domains (see PCT Publication No. WO 02/02773). In rDVD-Ig™ at least one of the variable domains comprises a ligand binding domain of a receptor (RD).

The terms multi-specific and multivalent IgG-like molecules or “pDVD-Ig™” are capable of binding two or more proteins (e.g., antigens). pDVD-Ig™ are described in U.S. Ser. No. 61/746,617, incorporated herein by reference in its entirety. In certain embodiments, pDVD-Ig™ are disclosed which are generated by specifically modifying and adapting several concepts. These concepts include but are not limited to: (1) forming Fc heterodimer using CH3 “knobs-into-holes” design, (2) reducing light chain missing pairing by using CH1/CL cross-over, and (3) pairing two separate half IgG molecules at protein production stage using “reduction then oxidation” approach.

In certain embodiments, the binding protein of the invention is a “half-DVD-Ig”™ derived from a DVD-Ig™. The half-DVD-Ig™ preferably does not promote cross-linking observed with naturally occurring antibodies which can result in antigen clustering and undesirable activities. See U.S. patent publication number 20120201746 published Aug. 9, 2012, and international publication number WO/2012/088302 published Jun. 28, 2012, each of which is incorporated by reference herein in its entirety.

In one embodiment, a pDVD-Ig™ construct may be created by combining two halves of different DVD-Ig™ molecules, or a half DVD-Ig™ and half IgG molecule. A pDVD-Ig™ construct may be expressed from four unique constructs to create a monovalent, multi-specific molecules through the use of heavy chain CH3 knobs-into-holes design. In another embodiment, a pDVD-Ig™ construct may contain two distinct light chains, and may utilize structural modifications on the Fc of one arm to ensure the proper pairing of the light chains with their respective heavy chains. In one aspect, the heavy chain constant region CH1 may be swapped with a light chain constant region hCk on one Fab. In another aspect, an entire light chain variable region, plus hCk, may be swapped with a heavy chain variable region, plus CH1. pDVD-Ig™ construct vectors that accommodate these unique structural requirements are also disclosed.

In some embodiments, pDVD-Ig™ contain four polypeptide chains, namely, first, second, third and fourth polypeptide chains. In one aspect, the first polypeptide chain may contain VD1-(X1)n-VD2-CH-(X2)n, wherein VD1 is a first heavy chain variable domain, VD2 is a second heavy chain variable domain, CH is a heavy chain constant domain, X1 is a linker with the proviso that it is not a constant domain, and X2 is an Fc region. In another aspect, the second polypeptide chain may contain VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region. In another aspect, the third polypeptide chain may contain VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region. In another aspect, the fourth polypeptide chain may contain VD3-(X3)n-VD4-CH-(X4)n, wherein VD3 is a third light chain variable domain, VD4 is a fourth light chain variable domain, CH is a heavy chain constant domain, X3 is a linker with the proviso that it is not a constant domain, and X4 does not comprise an Fc region. In another aspect, n is 0 or 1, and the VD1 domains on the first and second polypeptide chains form one functional binding site for antigen A, the VD2 domains on the first and second polypeptide chains form one functional binding site for antigen B, the VD3 domains on the third and fourth polypeptide chains form one functional binding site for antigen C, and the VD4 domains on the third and fourth polypeptide chains form one functional binding site for antigen D. In one embodiment, antigens A, B, C and D may be the same antigen, or they may each be a different antigen. In another embodiment, antigens A and B are the same antigen, and antigens C and D are the same antigen.

As used herein “monobody DVD-Ig™” or “mDVD-Ig™” refers to a class of binding molecules wherein one binding arm has been rendered non-functional. mDVD-Ig™ are described in U.S. Ser. No. 61/746,615, incorporated herein by reference in its entirety. In one aspect, mDVD-Ig™ possesses only one functional arm capable of binding a ligand. In another aspect, the one functional arm may have one or more binding domains for binding to different ligands. The ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and combinations thereof.

In one embodiment, mDVD-Ig™ contains four polypeptide chains, wherein two of the four polypeptide chains comprise VDH-(X1)n-C-(X2)n. In one aspect, VDH is a heavy chain variable domain, X1 is a linker with the proviso that it is not CH1, C is a heavy chain constant domain, X2 is an Fc region, and n is 0 or 1. The other two of the four polypeptide chains comprise VDL-(X3)n-C-(X4)n, wherein VDL is a light chain variable domain, X3 is a linker with the proviso that it is not CH1, C is a light chain constant domain, X4 does not comprise an Fc region, and n is 0 or 1. In another aspect, at least one of the four polypeptide chains comprises a mutation located in the variable domain, wherein the mutation inhibits the targeted binding between the specific antigen and the mutant binding domain. The Fc regions of the two polypeptide chains that have a formula of VDH-(X1)n-C-(X2)n may each contain a mutation, wherein the mutations on the two Fc regions enhance heterodimerization of the two polypeptide chains. In one aspect, knobs-into-holes mutations may be introduced into these Fc regions to achieve heterodimerization of the Fc regions. See Atwell et al. J. Mol. Biol. 1997, 270: 26-35.

As used herein “cross-over DVD-Ig™” or “coDVD-Ig™” refers to a DVD-Ig™ wherein the cross-over of variable domains is used to resolve the issue of affinity loss in the inner antigen-binding domains of some DVD-Ig™ molecules. coDVD-Ig™ are described in U.S. Ser. No. 61/746,619, incorporated herein by reference in its entirety. In certain specific embodiments, cross-over dual-variable-domain (DVD) Igs are generated by crossing over light chain and the heavy chain variable domains of a dual-variable-domain (DVD) Ig or Ig like protein. In another aspect, the length and sequence of the linkers linking the variable domains may be optimized for each format and antibody sequence/structure (frameworks) to achieve desirable properties. The disclosed concept and methodology may also be extended to Ig or Ig like proteins having more than two antigen binding domains.

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The term “linker” means an amino acid residue or a polypeptide comprising two or more amino acid residues joined by peptide bonds that are used to link two polypeptides (e.g., two VH or two VL domains). Such linker polypeptides are well known in the art (see, e.g., Holliger et al. (1993) Proc. Natl. Acad. Sci. USA 90:6444-6448; Poljak et al. (1994) Structure 2:1121-1123).

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). In addition single chain antibodies also include “linear antibodies” comprising a pair of tandem Fv segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As used herein, the terms “VH domain” and “VL domain” refer to single antibody variable heavy and light domains, respectively, comprising FR (Framework Regions) 1, 2, 3 and 4 and CDR (Complementary Determinant Regions) 1, 2 and 3 (see Kabat et al. (1991) Sequences of Proteins of Immunological Interest. (NIH Publication No. 91-3242, Bethesda).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a polypeptide and/or other determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (one prevents the binding or modulating effect of the other). In addition, structural definitions of epitopes (overlapping, similar, identical) are informative; and functional definitions encompass structural (binding) and functional (modulation, competition) parameters. Different regions of proteins may perform different functions. For example specific regions of a cytokine interact with its cytokine receptor to bring about receptor activation whereas other regions of the protein may be required for stabilizing the cytokine. To abrogate the negative effects of cytokine signaling, the cytokine may be targeted with a binding protein that binds specifically to the receptor interacting region(s), thereby preventing the binding of its receptor. Alternatively, a binding protein may target the regions responsible for cytokine stabilization, thereby designating the protein for degradation. The methods of visualizing and modeling epitope recognition are known to one skilled in the art (US 20090311253).

As used herein, the term “specifically binds to” refers to the ability of a binding polypeptide to bind to an antigen with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to an antigen with an affinity that is at least two-fold greater than its affinity for a nonspecific antigen. It shall be understood, however, that the binding polypeptide are capable of specifically binding to two or more antigens which are related in sequence. For example, the binding polypeptides of the invention can specifically bind to both human and a non-human (e.g., mouse or non-human primate) orthologos of an antigen.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “T-cell receptor (TCR) complex component” refers to cell surface molecule (e.g., a protein) that is a part of the T-cell receptor complex. Exemplary T-cell receptor (TCR) complex components include, without limitation, CD3γ, CD3δ, and CD3ε.

The term “target cell” refers to a cell that is recruited to an immune cell (e.g., a cytotoxic T-cell) using the binding proteins disclosed herein. In certain embodiments, the target cell is lysed by the recruited immune cell.

The term “cell surface modulator” refers to a cell surface molecule (e.g., a protein) on an immune cell that, when bound by a ligand (e.g., binding protein), can modulate (e.g., enhance or suppress) the activation of the immune cell (e.g., via immune cell ligation).

The term “T-cell modulator” refers to a cell surface molecule (e.g., a protein) on a T-cell that, when bound by a ligand (e.g., binding protein), can modulate (e.g., enhance or suppress) the activation of the T-cell (e.g., via TCR ligation).

As used herein, the term “co-stimulator”, refers to a cell surface molecule on an immune cell that, when engaged by a binding protein, enhances signaling through an immune receptor on the same immune cell.

As used herein, the term “co-repressor”, refers to a cell surface molecule on an immune cell that, when engaged by a binding protein, represses signaling through an immune receptor on the same immune cell.

The term “disease-associated antigen” refers to an antigen that is present on the surface of target cell that is associated with or causes the pathology of a disease or disorder, wherein the antigen can be used to recruit a T-cell using the binding proteins disclosed herein.

The term “tumor-associated antigen” refers to an antigen that is present on the surface of a tumor cell, wherein the antigen can be used to recruit a T-cell using the binding proteins disclosed herein.

The term “CD3γ” refers to the gamma chain of the surface glycoprotein CD3. Exemplary CD3γ proteins include the human CD3γ protein set forth in REFSEQ accession number NM_{—}000073.2.

The term “CD3δ” refers to the delta chain of the surface glycoprotein CD3. Exemplary CD3δ proteins include the human CD3δ protein set forth in REFSEQ accession number NM_{—}001040651.1.

The term “CD3ε” refers to the epsilon chain of the surface glycoprotein CD3. Exemplary CD3ε proteins include the human CD3ε protein set forth in REFSEQ accession number NM_{—}000733.3.

The term “CD2” refers to the cell adhesion molecule CD2, also referred to as “cluster of differentiation 2”, that is found on the surface of T cells and natural killer (NK) cells. Exemplary CD2 proteins include the human CD2 protein set forth in REFSEQ accession number NM_{—}001767.3.

The term “4-1BB” refers to a type 2 transmembrane glycoprotein belonging to the TNF superfamily (also referred to as TNFRSF9). Exemplary 4-1BB proteins include the human 4-1BB protein set forth in REFSEQ accession number NM_{—}001561.5.

The term “PD1” refers to “programmed cell death protein 1,” a cell surface protein encoded by the PDCD1 gene. Exemplary PD-1 proteins include the human PD-1 protein set forth in REFSEQ accession number NM_{—}005018.2.

The term “LAG3” refers to “lymphocyte-activation protein 3,” a protein that belongs to the immunoglobulin (Ig) superfamily. Exemplary LAG3 proteins include the human LAG3 protein set forth in REFSEQ accession number NM_{—}002286.5.

The term “CTLA4” refers to a protein receptor found on the surface of T cells. Exemplary CTLA4 proteins include the human CTLA4 protein set forth in REFSEQ accession number NM_{—}005214.4.

The term “GITR” refers to a receptor protein encoded by the TNFRSF18 gene. Exemplary GITR proteins include the human GITR protein set forth in REFSEQ accession number NM_{—}004195.2.

The term “EGFR” refers to a cell-surface receptor for members of the epidermal growth factor family (EGF-family) of extracellular protein ligands. Exemplary EGFR proteins include the human EGFR protein set forth in REFSEQ accession number NM_{—}005228.3.

The term “therapeutic combination” as used herein means a combination of one or more active drug substances, e.g., multispecific binding proteins. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

The terms “crystal” and “crystallized” refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, CRYSTALLIZATION OF NUCLEIC ACIDS AND PROTEINS, A PRACTICAL APPROACH, 2nd ea., pp. 20 1-16, Oxford University Press, New York, N.Y., (1999).

The term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (U.S. Patent Application Ser. No. 61/021,282) were used for parental antibody and DVD-binding protein cloning. V1, derived from pJP183; pHybE-hCg1,z,non-a V2, was used for cloning of antibody and DVD heavy chains with a wildtype constant region. V2, derived from pJP191; pHybE-hCk V3, was used for cloning of antibody and DVD light chains with a kappa constant region. V3, derived from pJP192; pHybE-hCl V2, was used for cloning of antibody and DVDs light chains with a lambda constant region. V4, built with a lambda signal peptide and a kappa constant region, was used for cloning of DVD light chains with a lambda-kappa hybrid V domain. V5, built with a kappa signal peptide and a lambda constant region, was used for cloning of DVD light chains with a kappa-lambda hybrid V domain. V7, derived from pJP183; pHybE-hCg1,z,non-a V2, was used for cloning of antibody and DVD heavy chains with a (234,235 AA) mutant constant region.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. Coli; mammalian cell lines CHO, HEK 293, COS, NS0, SP2 and PER.C6; the insect cell line Sf9; and the fungal cell Saccharomyces cerevisiae.

### II. MULTISPECIFIC BINDING PROTEINS

In one aspect the present disclosure provides a multispecific binding protein comprising a multispecific binding protein comprising a first binding site that specifically binds to a target cell antigen, a second binding site that specifically binds to a cell surface receptor on an immune cell, and a third binding site that specifically binds to cell surface modulator on the immune cell.

Any immune cell can be recruited to a target cell using the methods and compositions disclosed herein. In certain embodiments, the immune cell is a myeloid cell or a lymphoid cell. In certain embodiments, the immune cell is an effector cell. In certain embodiments, the immune cell is T-cell, macrophage, dendritic cell, natural killer cell or eosinophil. In certain embodiments, the immune cell is a cytotoxic T-cell. In certain embodiments, the cell surface receptor on the immune cell is a T-cell receptor (TCR) complex component.

In certain embodiments, the multispecific binding protein comprises a first binding site that specifically binds to a target cell antigen, a second binding site that specifically binds to a T-cell receptor (TCR) complex component on a T-cell, and a third binding site that specifically binds to a T-cell modulator on the T-cell.

Any cell-surface antigen on a target cell can be targeted in the methods and compositions disclosed herein. The target cell antigen can comprise a protein, carbohydrate or lipid, or combinations thereof. In certain embodiments, the target cell antigen is a cell surface protein. In certain embodiments, the antigen is a disease-associated antigen, e.g., a tumor-associated antigen. In certain embodiments, the target cell antigen is CD3, CD19, CD20, CD80; CD22, CD30, CD40, EGFR, HER2, HER3, HER4, IGF1, IGF12, IGF1R, RON, HGF, c-MET, VEGF, DLL4, NRP1, PLGF, EpCAM, CEA, PSMA, or TRAIL-R.

Any TCR complex component can be targeted in the methods and compositions disclosed herein. In certain embodiments, the TCR complex component is CD3γ, CD3δ, or CD3ε.

Any cell surface modulator (e.g., stimulator or inhibitor of immune cell activation) can be targeted in the methods and compositions disclosed herein. In certain embodiments, the modulator is CD2, 4-1BB, PD1, LAG3, CTLA4, GITR CD80, CD86, PD-L1, PD-L2, B7-H1, B7-H3, B7-H4, HVEM, ILT3, ILT4, BTLA, CD160, MHC-1, CD40, ICOSL, CD70, OX40L, 4-1BBL, GITRL, LIGHT, TIM3, TIM4, ICAM1, LFA3, CD28, CD40L, ICOS, CD27, OX40, Galectin 9, TIM1, DR3, CD30, SLAM, 2B4, TIGIT, CD226, CD160, LAIR1, CD96, CRTAM, CD226, CD352, CD319, BTN1, BTN2, BTN3, CD120a, CD120b, TNFRSF3, TNFRSF6B, TNFRSF10A, TNFRSF10B, TNFRSF10C, TNFRSF10D, TNFRSF19, TNFRSF19L, TNFRSF25, TNFRSF27, EDAR, LFA1, CD95, CD265, CD267, CD268, CD269, CD358, or CD271.

In certain embodiments, multispecific binding protein binds to EGFR on a target cell, and CD3ε and CD2 on a T-cell. In certain embodiments, multispecific binding protein binds to EGFR on a target cell, and CD3ε and 41BB on a T-cell.

In certain embodiments, multispecific binding protein comprises one or more of the CDR, VH, or VL sequences set forth in Tables 1-5 herein.

Any multispecific binding protein format can be employed. In certain embodiments, the multispecific binding protein is a DVD-Ig, half-DVD-Ig, a scDVD-Ig, an fDVD-Ig, an rDVD-Ig, a pDVD-Ig, an mDVD-Ig or a coDVD-Ig, as described herein.

In certain embodiments, the multispecific binding protein of is a pDVD-Ig having first, second, third and fourth polypeptide chains, wherein said first polypeptide chain comprises VD1-(X1)n-VD2-C-(X2)n, wherein VD1 is a first heavy chain variable domain, VD2 is a second heavy chain variable domain, C is a constant domain, X1 is a linker with the proviso that it is not a constant domain, and X2 is an Fc region; wherein said second polypeptide chain comprises VD3-(X1)n-VD4-C-(X2)n, wherein VD3 is a first light chain variable domain, VD4 is a second light chain variable domain, C is a constant domain, X1 is a linker with the proviso that it is not a constant domain, and X2 does not comprise an Fc region; wherein said third polypeptide chain comprises VD5-C-(X3)n, wherein VD5 is a third heavy chain variable domain, C is a constant domain, and X3 is an Fc region; wherein said fourth polypeptide chain comprises VD6-C-(X3)n, wherein VD6 is a third light chain variable domain, C is a constant domain, and X4 does not comprise an Fc region; wherein n is 0 or 1, and wherein the VD1 and VD3 domains on the first and second polypeptide chains form one functional binding site for a first antigen, the VD2 and VD4 domains on the first and second polypeptide chains form one functional binding site for a second antigen, and the VD5 and VD6 domains on the third and fourth polypeptide chains form one functional binding site for a third antigen. In certain embodiments, the Fc region of the first and third polypeptide chains each comprises a mutation, wherein said mutations on the two Fc regions enhance heterodimerization of the first and third polypeptide chains

In certain embodiments, the first antigen is the target cell antigen, the second antigen is the TCR complex component, and the third antigen is the T-cell modulator.

In certain embodiments, the pDVD-Ig multispecific binding protein comprises (a) VD1, VD2, or VD5 comprises HCDR1-3 regions selected from the group consisting of SEQ ID NO: 88, 89, and 90; 94, 95, and 96; 100, 101, and 102; and 106, 107, and 108; (b) VD1, VD2, and VD5 comprise HCDR1-3 regions selected from the group consisting of SEQ ID NO: 88, 89, and 90; 94, 95, and 96; 100, 101, and 102; and 106, 107, and 108; (c) VD1, VD2, or VD5 comprise a VH amino acid sequence selected from the group consisting of SEQ ID NO: 80, 82, 84, and 86; (d) VD2 and VD5 comprise SEQ ID NO: 86 and 80, respectively; (e) VD2 and VD5 comprise SEQ ID NO: 86 and 82, respectively; (f) VD1, VD2 and VD5 comprise SEQ ID NO: 84, 86 and 80, respectively; or (g) VD1, VD2 and VD5 comprise SEQ ID NO: 84, 86 and 82, respectively.

In certain embodiments, the pDVD-Ig multispecific binding protein comprises (a) VD3, VD4, or VD6 comprise LCDR1-3 regions selected from the group consisting of SEQ ID NO: 91, 92, and 93; 97, 98, and 99; 103, 104, and 105; and 109, 110, and 111; (b) VD3, VD4, and VD6 comprise LCDR1-3 regions selected from the group consisting of SEQ ID NO: 91, 92, and 93; 97, 98, and 99; 103, 104, and 105; and 109, 110, and 111; (c) VD3, VD4, or VD6 comprise a VL amino acid sequence selected from the group consisting of SEQ ID NO: 81, 83, 85, and 87; (d) VD3 and VD6 comprise SEQ ID NO: 87 and 81, respectively; (e) VD3 and VD6 comprise SEQ ID NO: 87 and 83, respectively; (f) VD3, VD4, and VD6 comprise SEQ ID NO: 84, 87 and 81, respectively; or (g) VD3, VD4, and VD6 comprise SEQ ID NO: 84, 87 and 83, respectively.

In certain embodiments, the pDVD-Ig multispecific binding protein comprises a polypeptide sequence selected from the group consisting of SEQ ID NO: 32-79.

In certain embodiments, the pDVD-Ig multispecific binding protein comprises the first, second, third and fourth polypeptide chains comprise the amino acid sequences set forth in SEQ ID NO: 32, 33, 34, and 35; 36, 37, 38, and 39; 40, 41, 42, and 43; 44, 45, 46, and 47; 48, 49, 50, and 51; 52, 53, 54, and 55; 56, 57, 58, and 59; 60, 61, 62, and 63; 64, 65, 66, and 67; 68, 69, 70, and 71; 72, 73, 74, and 75; 76, 77, 78, and 79, respectively.

In another aspect, provided is a therapeutic combination comprising: a first multispecific binding protein comprising a first binding site that specifically binds to a target cell antigen, and a second binding site that specifically binds to a T-cell receptor (TCR) complex component on a T-cell; and a second multispecific binding protein comprising a first binding site that specifically binds to a target cell antigen, and a second binding site that specifically binds to a T-cell modulator on the T-cell.

Any target cell antigen can be targeted in the methods and compositions disclosed herein. In certain embodiments, the antigen is a disease-associated antigen, e.g., a tumor-associated antigen (e.g., EGFR).

Any TCR complex component can be targeted in the methods and compositions disclosed herein. In certain embodiments, the TCR complex component is CD3γ, CD3δ, or CD3ε.

Any T-cell modulator (e.g., stimulator or inhibitor of T-cell activation) can be targeted in the methods and compositions disclosed herein. In certain embodiments, the T-cell modulator is CD2, 41BB, PD1, LAG3, CTLA4 and/or GITR

In certain embodiments, multispecific binding protein binds to EGFR on a target cell, and CD3ε and CD2 on a T-cell. In certain embodiments, multispecific binding protein binds to EGFR on a target cell, and CD3ε and 41BB on a T-cell.

Any multispecific binding protein format can be employed. In certain embodiments, the multispecific binding protein is a DVD-Ig, half-DVD-Ig, a scDVD-Ig, an fDVD-Ig, an rDVD-Ig, a pDVD-Ig, an mDVD-Ig or a coDVD-Ig, as described herein.

In certain embodiments of the therapeutic combination, the first multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 100, 101, 102; 106, 107, and 108; (b) VD1 or VD2 comprises HCDR1-3 regions selected from the group consisting of SEQ ID NO: 100, 101, 102; 106, 107, and 108; (c) VD1 and VD2 comprise HCDR1-3 regions selected from the group consisting of SEQ ID NO: 100, 101, 102; 106, 107, and 108; (d) VD1 or VD2 comprise a VH amino acid sequence selected from the group consisting of SEQ ID NO: 84 and 86; (e) VD1 and VD2 comprise SEQ ID NO: 84 and 86, respectively.

In certain embodiments of the therapeutic combination, the first multispecific binding protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 103, 104, 105, 109, 110, and 111; (b) VD1 or VD2 comprises LCDR1-3 regions selected from the group consisting of SEQ ID NO: 103, 104, and 105; and 109, 110, and 111; (c) VD1 and VD2 comprise LCDR1-3 regions selected from the group consisting of SEQ ID NO: 103, 104, and 105; and 109, 110, and 111; (d) VD1 or VD2 comprise a VL amino acid sequence selected from the group consisting of SEQ ID NO: 85, and 87; (e) VD1 and VD2 comprise SEQ ID NO: 85 and 87, respectively.

In certain embodiments of the therapeutic combination, the second multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 88, 89, 90; 94, 95, 96; 100, 101, and 102; (b) VD1 or VD2 comprises HCDR1-3 regions selected from the group consisting of SEQ ID NO: 88, 89, and 90; 94, 95, and 96; and 100, 101, and 102; (c) VD1 and VD2 comprise HCDR1-3 regions selected from the group consisting of SEQ ID NO: 88, 89, and 90; 94, 95, and 96; and 100, 101, and 102; (d) VD1 or VD2 comprise a VH amino acid sequence selected from the group consisting of SEQ ID NO: 80, 82, and 84; (e) VD1 and VD2 comprise SEQ ID NO: 80 and 84, respectively; or (f) VD2 and VD2 comprise SEQ ID NO: 82 and 84, respectively.

In certain embodiments of the therapeutic combination, the second multispecific binding protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 91, 92, 93; 97, 98, 99; 103, 104, and 105; (b) VD1 or VD2 comprises LCDR1-3 regions selected from the group consisting of SEQ ID NO: 91, 92, and 93; 97, 98, and 99; and 103, 104, and 105; (c) VD1 and VD2 comprise LCDR1-3 regions selected from the group consisting of SEQ ID NO: 91, 92, and 93; 97, 98, and 99; and 103, 104, and 105; (d) VD1 or VD2 comprise a VL amino acid sequence selected from the group consisting of SEQ ID NO: 81, 83, and 87; (e) VD1 and VD2 comprise SEQ ID NO: 81 and 85, respectively; or (f) VD2 and VD2 comprise SEQ ID NO: 83 and 85, respectively.

In another aspect, the instant disclosure provides a multispecific binding protein comprising: a first binding site that specifically binds to a first modulator; and a second binding site that specifically binds to a second modulator.

In certain embodiments, the first or second modulator is a stimulator of T-cell activation. In certain embodiments, the first and second modulator is a stimulator of T-cell activation. In certain embodiments, the first or second modulator is an inhibitor of T-cell activation. In certain embodiments, the first and second modulator is an inhibitor of T-cell activation. In certain embodiments, the first or second modulator is CD2, 4-1BB, PD1, LAG3, CTLA4, GITR CD80, CD86, PD-L1, PD-L2, B7-H1, B7-H3, B7-H4, HVEM, ILT3, ILT4, BTLA, CD160, MHC-1, CD40, ICOSL, CD70, OX40L, 4-1BBL, GITRL, LIGHT, TIM3, TIM4, ICAM1, LFA3, CD28, CD40L, ICOS, CD27, OX40, Galectin 9, TIM1, DR3, CD30, SLAM, 2B4, TIGIT, CD226, CD160, LAIR1, CD96, CRTAM, CD226, CD352, CD319, BTN1, BTN2, BTN3, CD120a, CD120b, TNFRSF3, TNFRSF6B, TNFRSF10A, TNFRSF10B, TNFRSF10C, TNFRSF10D, TNFRSF19, TNFRSF19L, TNFRSF25, TNFRSF27, EDAR, LFA1, CD95, CD265, CD267, CD268, CD269, CD358, or CD271.

In certain embodiments, the first T-cell modulator is 41BB and the second T-cell modulator is LAG3. In certain embodiments, the first T-cell modulator is PD1 and the second T-cell modulator is LAG3. In certain embodiments, the first T-cell modulator is CTLA4 and the second T-cell modulator is LAG3. In certain embodiments, the first T-cell modulator is GITR and the second T-cell modulator is LAG3. In certain embodiments, the first T-cell modulator is PD1 and the second T-cell modulator is PD1, wherein the first and second binding site binds to different epitopes of PD1. In certain embodiments, the first T-cell modulator is PD1 and the second T-cell modulator is CTLA4.

In certain embodiments, the multispecific binding protein is a DVD-Ig, half-DVD-Ig, a scDVD-Ig, an fDVD-Ig, an rDVD-Ig, a pDVD-Ig, an mDVD-Ig or a coDVD-Ig. In certain embodiments, the multispecific binding protein comprises a polypeptide chain having the formula VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; (X1)n is (X1)0 or (X1)1; (X2)n is (X2)0 or (X2)1; and wherein (a) VD1 or VD2 comprise a CDR region amino acid sequence selected from SEQ ID NO: 120, 121, 122, 126, 127, 128, 132, 134, 134, 138, 139, 140; (b) VD1 or VD2 comprises HCDR1-3 regions selected from the group consisting of SEQ ID NO: 120, 121, and 122; 126, 127, and 128; 132, 134, and 134, and 138, 139, and 140; (c) VD1 and VD2 comprise HCDR1-3 regions selected from the group consisting of SEQ ID NO: 120, 121, and 122; 126, 127, and 128; 132, 134, and 134, and 138, 139, and 140; (d) VD1 or VD2 comprise a VH amino acid sequence selected from the group consisting of SEQ ID NO: 112, 114, 115, 116, 118; (e) VD1 and VD2 comprise a VH amino acid sequence selected from the group consisting of SEQ ID NO: 112, 114, 115, 116, 118; or (f) the polypeptide chain comprises an amino acid sequence selected from the group consisting of SEQ ID NO: 144, 146, 148, 150, 152, 154, 156, and 158.

In certain embodiments, the multispecific binding protein comprises a polypeptide chain a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not CL; X2 does not comprise an Fc region; (X1)n is (X1)0 or (X1)1; (X2)n is (X2)0 or (X2)1; and wherein (a) VD1 or VD2 comprise a CDR region amino acid sequence selected from SEQ ID NO: 123, 124, 125, 129, 130, 131, 135, 136, 137, 141, 142, 143; (b) VD1 or VD2 comprises HCDR1-3 regions selected from the group consisting of SEQ ID NO: 123, 124, and 125; 129, 130, and 131; 135, 136, and 137; and 141, 142, and 143; (c) VD1 and VD2 comprise HCDR1-3 regions selected from the group consisting of SEQ ID NO: 123, 124, and 125; 129, 130, and 131; 135, 136, and 137; and 141, 142, and 143; (d) VD1 or VD2 comprise a VL amino acid sequence selected from the group consisting of SEQ ID NO: 113, 115, 117, and 119; (e) VD1 and VD2 comprise a VL amino acid sequence selected from the group consisting of SEQ ID NO: 113, 115, 117, and 119; or (f) the polypeptide chain comprises an amino acid sequence selected from the group consisting of SEQ ID NO: 145, 147, 149, 151, 153, 155, 157, and 159.

In certain embodiments, the multispecific binding protein embodiments disclosed herein comprise at least one linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); or G/S based sequences (e.g., G4S repeats; SEQ ID NO: 29). In an embodiment, X2 is an Fc region. In another embodiment, X2 is a variant Fc region.

In certain embodiments, the multispecific binding protein embodiments disclosed herein comprise a linker comprising GS-H10 (Chain H) GGGGSGGGGS (SEQ ID NO:30). In various embodiments, the linker comprises GS-L10 (Chain L) GGSGGGGSG (SEQ ID NO:31). In various embodiments, the linker comprises HG-short (Chain H) ASTKGP (SEQ ID NO:21). In various embodiments, the linker comprises LK-long (Chain L) TVAAPSVFIFPP (SEQ ID NO: 14). For example SEQ ID NOs: 21 and 30 are located on a variable heavy chain or domain of a DVD-Ig. For example SEQ ID NOs: 14 and 31 are located on a variable light chain or domain of a DVD-Ig.

In certain embodiments, the binding protein peptide is recombinantly produced. In certain embodiments, the recombinant binding protein is encoded by a nucleotide sequence or the binding protein includes an amino acid sequence that is substantially identical or homologous to the sequences described herein, for example a sequence shown in any of the Examples and Tables herein. For example, recombinant binding protein or peptide is engineered and constructed using any of the sequences described herein. In a related embodiment, the binding protein or peptide is administered to a subject using a vector carrying a nucleotide sequence that encodes the binding protein or peptide. In various embodiments, the binding protein or peptide (with or without an agent) is delivered for example using a liposome, a lipid/polycation (LPD), a peptide, a nanoparticle, a gold particle, and a polymer.

In certain embodiments, the binding protein or peptide includes an amino acid sequence having a conservative sequence modification from the sequences shown herein, e.g., Tables 1-5. The phrase “conservative sequence modifications” refers to amino acid modifications that do not significantly affect or alter the characteristics (e.g., binding, stability, and orientation) of the binding protein, e.g., amino acid sequences of binding protein that present a side chain at the same relative position to allow for function in a manner similar to an unmodified binding protein. A conservative modification includes for example a substitution, addition, or deletion in the amino acid sequence of the binding protein or peptide. Modification of the amino acid sequence of recombinant multimeric binding protein is achieved using any known technique in the art e.g., site-directed mutagenesis or PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. Conservative amino acid substitutions are modifications in which the amino acid residue is replaced with an amino acid residue having a similar side chain such as replacing a small amino acid with a different small amino acid, a hydrophilic amino acid with a different hydrophilic amino acid, etc.

In certain embodiments, the multivalent binding protein has a molecular weight of greater than 150 kilodaltons (kD). In other embodiments, the binding protein has a molecular weight between 150 kD and 1000 kD. In other embodiments, the binding protein has a molecular weight between 150 kD and 500 kD, 150kD and 350 kD, 150 kD and 250 kD and 150 kD and 750 kD. In other embodiments, the binding protein has a molecular weight of greater than 150, 200, 250, 300, 450, 500, 550, 600, 650, 700, 750, 800, 850, 900, 950, 1000, 1050, 1100, 1150, 1200, 1250, 1300, 1350, 1400, 1450 and 1500 kD.

### III. USES OF BINDING PROTEINS

The multispecific binding protein disclosed herein are particularly useful for modulating activation state of an effector cell (e.g., a cytotoxic T-cell). Accordingly, also provided are methods of modulating an immune response (e.g., cell killing by cytotoxic T-cells) in a subject (e.g., a human subject). Accordingly, in another aspect, the instant disclosure provides is a method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein.

In another aspect, the instant disclosure provides a method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein, wherein the multispecific binding protein induces less cytokine production in the subject compared to administration of a equivalent effective amount of a therapeutic combination as disclosed herein.

In another aspect, the instant disclosure provides method of killing a target cell in a subject comprising administering to the subject an effective amount of a multispecific binding protein comprising a first binding site that specifically binds to a target cell antigen, a second binding site that specifically binds to a T-cell receptor (TCR) complex component on a T-cell, and a third binding site that specifically binds to a T-cell modulator on the T-cell, as disclosed herein, wherein the multispecific binding protein induces greater T-cell activation in the subject compared to administration of a equivalent effective amount of a therapeutic combination as disclosed herein.

The methods and compositions disclosed herein are suitable for any mammalian subject e.g., mice, rats, gerbils, hamsters, rabbits, apes, monkeys, humans, dogs, cats, camels, llamas, cattle and horses. In certain embodiments, the subject is a human subject.

In another aspect, the instant disclosure provides a method of modulating the activation state of a T-cell, the method comprising contacting the T-cell with a multispecific comprising: a first binding site that specifically binds to a first T-cell modulator; and a second binding site that specifically binds to a second T-cell modulator, as disclosed herein. In certain embodiments, the first and second T-cell modulator is a stimulator of T-cell activation, wherein the multispecific binding protein inhibits T-cell activation. In certain embodiments, the first and second T-cell modulator is an inhibitor of T-cell activation, wherein the T-cell exhibits activation.

In another aspect, the instant disclosure provides a method of directing a cytotoxic T-cell to lyse a target cell in a subject, the method comprising administering to the subject an effective amount of a multispecific comprising a first binding site that specifically binds to a first T-cell modulator, and a second binding site that specifically binds to a second T-cell modulator, as disclosed herein. Any target cell can be lysed using the methods disclosed herein. In certain embodiments, the target cell is a tumor cell.

In another aspect, the instant disclosure provides a method of reducing an inflammatory state in a subject, the method comprising administering to the an effective amount of a multispecific comprising a first binding site that specifically binds to a first T-cell modulator, and a second binding site that specifically binds to a second T-cell modulator, as disclosed herein. Any an inflammatory state can be reduced using the methods disclosed herein. In certain embodiments, the inflammatory state is caused by effector cell (e.g., cytotoxic T-cell) activation.

In another aspect, the invention provides a method of identifying a multispecific binding protein that modulates a T-cell response, the method comprising: contacting a population of cytotoxic T-cells with a population of target cells in the presence and absence of a multispecific binding protein, wherein the multispecific binding protein simultaneously and specifically binds to a cell surface co-stimulator or co-repressor on T-cells and to a cell surface antigen on the target cells; and measuring the amount cell killing of the population of target cells, wherein an increase or decrease in cell killing in the presence of the multispecific binding, compared to the absence of the multispecific binding protein, identifies the multispecific binding protein as modulator of a T-cell response.

In another aspect, the invention provides a method of identifying a multispecific binding protein that modulates a T-cell response, the method comprising: contacting a population of cytotoxic T-cells with a population of antigen presenting cells in the presence and absence of a multispecific binding protein, wherein the multispecific binding protein simultaneously and specifically binds to two cell surface co-stimulators or co-repressors on T-cells; and measuring the amount of cytokine released from the population of T-cells, wherein an increase or decrease in amount of cytokine released in the presence of the multispecific binding, compared to the absence of the multispecific binding protein, identifies the multispecific binding protein as modulator of a T-cell response.

In another aspect, the invention provides a method of modulating an immune response in a subject, the method comprising: administering a therapeutic amount of a multispecific binding protein that simultaneously and specifically binds to a cell surface co-stimulator or co-repressor on an immune cell (e.g., a T-cell) and to a cell surface antigen on a target cell, wherein the multispecific binding protein modulates a normal response of the immune cell (e.g., T-cell) to target cell binding.

### IV. GENERATION OF BINDING PROTEINS

The multispecific binding protein can be generated using various techniques. Expression vectors, host cell and methods of generating the binding protein are provided and are well known in the art.

Generation of Parent Monoclonal Antibodies

The variable domains of the DVD binding protein can be obtained from parent antibodies, including polyclonal Abs and mAbs capable of binding antigens of interest. These antibodies may be naturally occurring or may be generated by recombinant technology. The person of ordinary skill in the art is well familiar with many methods for producing antibodies, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanized antibodies. See, e.g., US Patent Publication No. 20090311253 A1. Variable domains may also be prepared using affinity maturation techniques.

Criteria for Selecting Parent Monoclonal Antibodies

An embodiment is provided comprising selecting parent antibodies with at least one or more properties desired in the DVD binding protein molecule. In an embodiment, the desired property is one or more antibody parameters, such as, for example, antigen specificity, affinity to antigen, potency, biological function, epitope recognition, stability, solubility, production efficiency, immunogenicity, pharmacokinetics, bio availability, tissue cross reactivity, or orthologous antigen binding. See, e.g., US Patent Publication No. 20090311253.

Construction of Binding Protein Molecules

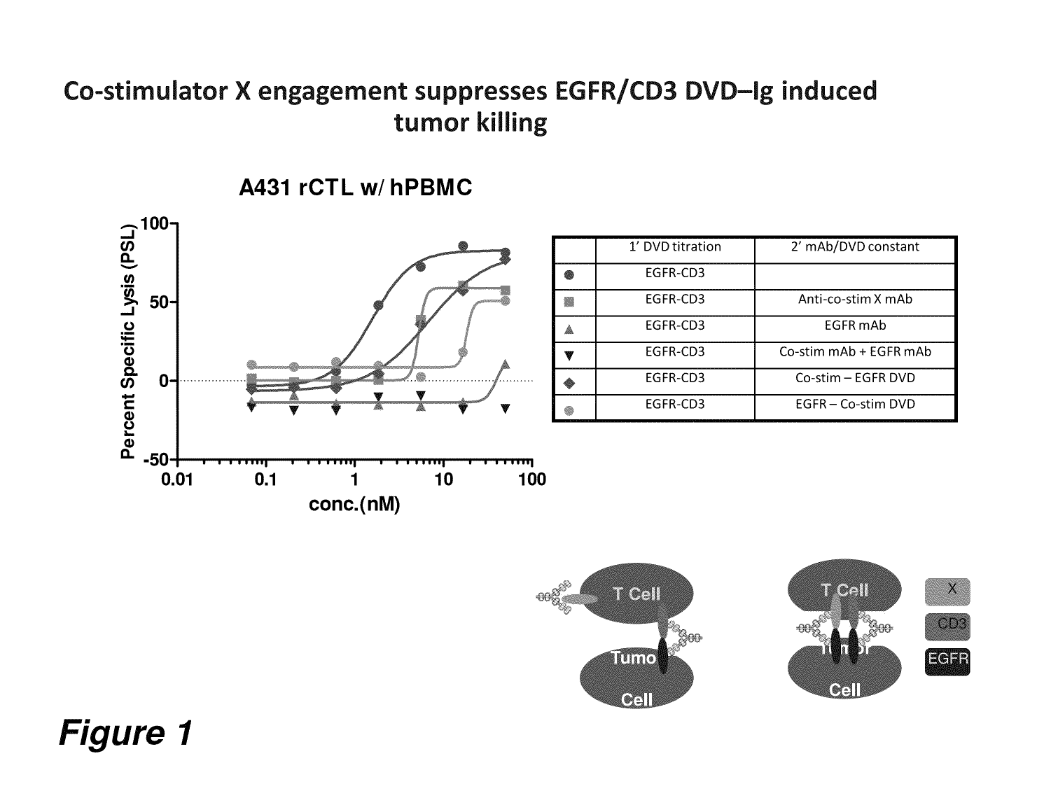

The binding protein may be designed such that two different light chain variable domains (VL) from the two different parent monoclonal antibodies are linked in tandem directly or via a linker by recombinant DNA techniques, followed by the light chain constant domain CL. Similarly, the heavy chain comprises two different heavy chain variable domains (VH) linked in tandem, directly or via a linker, followed by the constant domain CH1 and Fc region (FIG. 1).

The variable domains can be obtained using recombinant DNA techniques from parent antibodies generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

The linker sequence may be a single amino acid or a polypeptide sequence. In an embodiment, the choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from the C-terminus of a V domain and 4-6 residues from the N-terminus of a CL/CH1 domain. DVD binding proteins were generated using N-terminal 5-6 amino acid residues, or 11-12 amino acid residues, of CL or CH1 as a linker in the light chain and heavy chains, respectively. The N-terminal residues of CL or CH1 domains, particularly the first 5-6 amino acid residues, can adopt a loop conformation without strong secondary structures, and therefore can act as flexible linkers between the two variable domains. The N-terminal residues of CL or CH1 domains are natural extension of the variable domains, as they are part of the Ig sequences, and therefore their use minimizes to a large extent any immunogenicity potentially arising from the linkers and junctions.

In a further embodiment, of any of the heavy chain, light chain, two chain, or four chain embodiments, includes at least one linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); or G/S based sequences (e.g., G4S repeats; SEQ ID NO: 29). In an embodiment, X2 is an Fc region. In another embodiment, X2 is a variant Fc region.

In various embodiments, the linker comprises GS-H10 (Chain H) GGGGSGGGGS (SEQ ID NO:30). In various embodiments, the linker comprises GS-L10 (Chain L) GGSGGGGSG (SEQ ID NO:31). In various embodiments, the linker comprises HG-short (Chain H) ASTKGP (SEQ ID NO:21). In various embodiments, the linker comprises LK-long (Chain L) TVAAPSVFIFPP (SEQ ID NO: 14). For example SEQ ID NOs: 21 and 30 are located on a variable heavy chain or domain of a DVD-Ig. For example SEQ ID NOs: 14 and 31 are located on a variable light chain or domain of a DVD-Ig.

Other linker sequences may include any sequence of any length of a CL/CH1 domain but not all residues of a CL/CH1 domain; for example the first 5-12 amino acid residues of a CL/CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); G/S based sequences (e.g., G4S repeats; SEQ ID NO: 29); hinge region-derived sequences; and other natural sequences from other proteins.

In an embodiment, a constant domain is linked to the two linked variable domains using recombinant DNA techniques. In an embodiment, a sequence comprising linked heavy chain variable domains is linked to a heavy chain constant domain and a sequence comprising linked light chain variable domains is linked to a light chain constant domain. In an embodiment, the constant domains are human heavy chain constant domains and human light chain constant domains respectively. In an embodiment, the DVD heavy chain is further linked to an Fc region. The Fc region may be a native sequence Fc region or a variant Fc region. In another embodiment, the Fc region is a human Fc region. In another embodiment, the Fc region includes Fc region from IgG1, IgG2, IgG3, IgG4, IgA, IgM, IgE, or IgD.

In another embodiment, two heavy chain DVD polypeptides and two light chain DVD polypeptides are combined to form a DVD binding protein. Tables 1-5 list amino acid sequences of VH and VL regions of exemplary antibodies useful for treating disease. In an embodiment, a DVD comprising at least two of the VH and/or VL regions listed in Tables 1-5, in any orientation, is provided. In some embodiments, VD1 and VD2 are independently chosen. The VH and VL domain sequences provided below comprise complementarity determining regions (CDRs) and framework sequences that are either known in the art or readily discernible using methods known in the art. In some embodiments, one or more of these CDRs and/or framework sequences are replaced, without loss of function, by other CDRs and/or framework sequences from binding proteins that are known in the art to bind to the same antigen. Detailed description of specific DVD binding proteins capable of binding specific targets, and methods of making the same, is provided in the Examples section below.

Production of Binding Proteins

The binding proteins provided herein may be produced by any of a number of techniques known in the art. For example, expression from host cells, wherein expression vector(s) encoding the DVD heavy and DVD light chains is (are) transfected into a host cell by standard techniques. Although it is possible to express the DVD binding proteins provided herein in either prokaryotic or eukaryotic host cells, DVD binding proteins are expressed in eukaryotic cells, for example, mammalian host cells, because such eukaryotic cells (and in particular mammalian cells) are more likely than prokaryotic cells to assemble and secrete a properly folded and immunologically active DVD binding protein.

In an exemplary system for recombinant expression of DVD proteins, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. A method of synthesizing a DVD protein provided herein by culturing a host cell provided herein in a suitable culture medium until a DVD protein is synthesized is also provided. The method can further comprise isolating the DVD protein from the culture medium.

An important feature of DVD binding protein is that it can be produced and purified in a similar way as a conventional antibody. The production of DVD binding protein results in a homogeneous, single major product with desired dual-specific activity, without the need for sequence modification of the constant region or chemical modifications. Other previously described methods to generate “bi-specific”, “multi-specific”, and “multi-specific multivalent” full length binding proteins can lead to the intracellular or secreted production of a mixture of assembled inactive, mono-specific, multi-specific, multivalent, full length binding proteins, and multivalent full length binding proteins with a combination of different binding sites.

Surprisingly, the design of the “dual-specific multivalent full length binding proteins” provided herein leads to a dual variable domain light chain and a dual variable domain heavy chain that assemble primarily to the desired “dual-specific multivalent full length binding proteins”.

At least 50%, at least 75% and at least 90% of the assembled, and expressed dual variable domain immunoglobulin molecules are the desired dual-specific tetravalent protein, and therefore possess enhanced commercial utility. Thus, a method to express a dual variable domain light chain and a dual variable domain heavy chain in a single cell leading to a single primary product of a “dual-specific tetravalent full length binding protein” is provided.

Methods of expressing a dual variable domain light chain and a dual variable domain heavy chain in a single cell leading to a “primary product” of a “dual-specific tetravalent full length binding protein”, where the “primary product” is more than 50%, such as more than 75% and more than 90%, of all assembled protein, comprising a dual variable domain light chain and a dual variable domain heavy chain are provided.

DVD Cassettes

In certain embodiments, cassettes can be used to construct binding proteins that specifically bind to an antigen expressed on brain vascular epithelium of a subject that facilitates uptake of the binding protein into the brain of the subject. In some embodiments, the formula for these binding proteins is

Out1-(X1)m-In1-(X2)n (I)

According to Formula I, Out1 is a first outer binding domain and In1 is a first inner binding domain. In certain embodiments, the inner binding domain represents a binding domain positioned closer to the Fc region of a DVD-Ig™ than the outer binding domain. In other embodiments, the outer binding domain is located at or near the N-terminal end of the binding protein while the inner binding domain is located at or near the C-terminal end of the binding protein.

According to Formula I, X1 is a linker. According to some embodiments, X1 is any of the linkers defined herein. According to other specific embodiments, X1 has a sequence comprising the amino acid sequences of SEQ ID NO:14 or 21 when Out1 specifically binds an antigen expressed on brain vascular epithelium of a subject that facilitates uptake of the binding protein into the brain of the subject and In1 does not specifically bind said antigen, while X1 has a sequence comprising the amino acid sequence of SEQ ID NO:30 or 31 when In1 specifically binds an antigen expressed on brain vascular epithelium of a subject that facilitates uptake of the binding protein into the brain of the subject and Out1 does not specifically bind said antigen. According to Formula I, X2 is an Fc region. The values of m and n in Formula I are 0 or 1. In certain embodiments, when n is 0 X1 is X1 comprises the amino acid sequence of SEQ ID NO:14 or 31. When n is 1 X1 comprises the amino acid sequences of SEQ ID NO:21 or 30 depending on whether Out1 or In1 specifically binds said antigen.

In other embodiments, a binding protein may comprise a second binding protein. In some embodiments, the formula for this second binding protein is

Out2-(X1)m-In2-(X2)n (II)

According to Formula II, Out2 is a second outer binding domain and In2 is a second inner binding domain. As explained above. in certain embodiments, the inner binding domain represents a binding domain positioned closer to the Fc region of a DVD-Ig™ than the outer binding domain. In other embodiments, the outer binding domain is located at or near the N-terminal end of the binding protein while the inner binding domain is located at or near the C-terminal end of the binding protein. X1 and X2 are as defined in Formula I, above.

Out2 and In2 operate in the same manner as Out1 and In1 described above. This second binding protein can be associated with a first binding protein to form a binding polypeptide such as a DVD-Ig™. In these embodiments, in the first binding protein n is 1 and in the second binding protein n is 0. In certain embodiments, both Out1 and Out2 bind an antigen expressed on brain vascular epithelium of a subject that facilitates uptake of the binding protein into the brain of the subject. In other embodiments, both In1 and In2 bind said antigen. According to other embodiments, Out1 and In2 or Out2 and In1 bind said antigen.

### V. OTHER USES OF BINDING PROTEINS

Given their ability to bind to two or more antigens the binding proteins provided herein can be used to detect the antigens (e.g., in a biological sample, such as serum or plasma), using a conventional immunoassay, such as an enzyme linked immunosorbent assays (ELISA), a radioimmunoassay (RIA), or tissue immunohistochemistry. The binding protein is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol and examples of suitable radioactive materials include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In an embodiment, the binding proteins provided herein are capable of neutralizing the activity of their antigen targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In another embodiment, a method for reducing antigen activity in a subject suffering from a disease or disorder in which the antigen activity is detrimental is provided. A binding protein provided herein can be administered to a human subject for therapeutic purposes.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins.

DVD binding proteins are useful as therapeutic agents to simultaneously block two different targets to enhance efficacy/safety and/or increase patient coverage.

Furthermore, DVD binding protein can be designed to either be physically linked to medical devices implanted into patients or target these medical devices (see Burke et al. (2006) Advanced Drug Deliv. Rev. 58(3): 437-446; Hildebrand et al. (2006) Surface and Coatings Technol. 200(22-23): 6318-6324; Drug/device combinations for local drug therapies and infection prophylaxis, Wu (2006) Biomaterials 27(11):2450-2467; Mediation of the cytokine network in the implantation of orthopedic devices, Marques (2005) Biodegradable Systems in Tissue Engineer. Regen. Med. 377-397). Briefly, directing appropriate types of cell to the site of medical implant may promote healing and restoring normal tissue function. Alternatively, inhibition of mediators (including but not limited to cytokines), released upon device implantation by a DVD coupled to or target to a device is also provided.

Binding protein molecules provided herein are useful as therapeutic molecules to treat various diseases, e.g., wherein the targets that are recognized by the binding proteins are detrimental. Such binding proteins may bind one or more targets involved in a specific disease. Without limiting the disclosure, further information on certain disease conditions is provided.

Neurodegenerative Diseases

Neurodegenerative diseases are either chronic in which case they are usually age-dependent or acute (e.g., stroke, traumatic brain injury, spinal cord injury, etc.). They are characterized by progressive loss of neuronal functions (e.g., neuronal cell death, axon loss, neuritic dystrophy, demyelination), loss of mobility and loss of memory. These chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Specific therapies targeting more than one disease mediator may provide even better therapeutic efficacy for chronic neurodegenerative diseases than observed with targeting a single disease mechanism (see Deane et al. (2003) Nature Med. 9:907-13; and Masliah et al. (2005) Neuron. 46:857).

In certain embodiments, the therapeutics bind one or more targets involved in chronic neurodegenerative diseases such as Alzheimer's disease. The efficacy of binding protein molecules and its combination with other therapeutics can be validated in pre-clinical animal models such as the transgenic mice that over-express amyloid precursor protein or RAGE and develop Alzheimer's disease-like symptoms. In addition, binding protein molecules can be constructed and tested for efficacy in the animal models and the best therapeutic binding protein can be selected for testing in human patients. Binding protein molecules can also be employed for treatment of other neurodegenerative diseases such as Parkinson's disease. Other pain related targets include CGRP, TNFα, RGMA, Substance P, Bradykinin, Nav1.7, LPA, P2X3, and NGF.

Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries and the primary injury is usually followed by secondary injury mechanisms (inflammatory mediators e.g., cytokines and chemokines) that worsen the initial injury and result in significant enlargement of the lesion area, sometimes more than 10-fold. The efficacy of binding protein molecules can be validated in pre-clinical animal models of spinal cord injury.

Other Disease Targets

Other disease targets or disease conditions which may be treated with the binding molecules of the invention are disclosed in US 2009-0304693A1 and US 2010-0076178A1, each of which are specifically incorporated by reference herein in their entireties. For example, a binding protein of the invention suitable for neurological use may bind at least one target antigen selected from the group consisting of Abeta; TNF-alpha; BACE1; IL-1.beta; IGF1,2; IL-18; IL-6; RAGE; NGF; EGFR; CD-20 and RGMA. In other exemplary embodiments, a binding protein of the disclosure is suitable for anti-cancer use and binds at least one target antigen selected from the group consisting of CD3, CD19, CD20, CD80; CD22, CD30, CD40, EGFR, HER2, HER3, HER4, IGF1, IGF12, IGF1R, RON, HGF, c-MET, VEGF, DLL4, NRP1, PLGF, EpCAM, CEA, PSMA, and TRAIL-R.

Pain Modulation

Brain pathways governing the perception of pain and the signals sent to and received from the body still not completely understood. Junctions in the spinal cord are involved in the relay and modulation of sensations of pain to various regions of the brain, including the periaqueductal grey region (Ugeer, P. L., Eccles, J. C., and Ugeer, E. G. (1987). Molecular Neurobiology of the Mammalian Brain, Plenum Press, New York).

Pain can be classified as either acute or chronic. Acute pain can be caused by damage to tissue and generally has a sudden onset and a limited duration. Chronic pain tends to last longer than acute pain and is usually associated with a long-term illness. It is usually more resistant to treatment, and can be the defining characteristic of a disease (such as fibromyalgia). It can be the result of damaged tissue, but more often is attributed to nerve damage. Pain can also be classified by the kind of damage that causes it. Nociceptive pain is pain caused by tissue damage, while neuropathic pain is pain caused by nerve damage. Nociceptive pain may be further divided into three different sub-categories: visceral, deep somatic, and superficial somatic pain.

Examples of pain include but are not limited to: acute pain, chronic pain, muscle pain, joint pain, chest pain, neck pain, shoulder pain, hip pain, abdominal pain, carpal tunnel syndrome, knee pain, back pain, myofascial pain syndrome, fibromyalgia, arthritic pain, headache (e.g., a migraine headache), Piriformis syndrome, whiplash, chronic muscle pain, nociceptive pain, visceral pain, deep somatic pain, superficial somatic pain, neuropathic pain, central pain syndrome, complex regional pain syndrome, diabetic peripheral neuropathy, pain associated with shingles, postherpetic neuralgia, neuralgia, trigeminal neuralgia, sciatica pain, arachnoiditis (spinal pain), central pain syndrome, phantom limb pain, phantom body pain, neuropathy, compartment syndrome, acute herpetic pain, post herpetic pain, causalgia pain, idiopathic pain, inflammatory pain, cancer pain, postoperative pain, interstitial cystitis pain, irritable bowel syndrome (IBS), tendinitis, breakthrough pain, and incident pain.

Neuropathic pain is a particular type of chronic pain that has a complex and variable etiology. It is frequently a chronic condition attributable to complete or partial transection of a nerve, trauma or injury to a nerve, nerve plexus or soft tissue, or other conditions, including cancer, AIDS and idiopathic causes. Neuropathic pain is characterized by hyperalgesia (lowered pain threshold and enhanced pain perception) and by allodynia (pain from innocuous mechanical or thermal stimuli). The condition is often progressive in nature. Because the hyperesthetic component of neuropathic pain does not respond to the same pharmaceutical interventions as does more generalized and acute forms of pain, development of effective long-term treatment modalities has been problematic.

Psychogenic pain is a condition associated or correlated with a psychological, emotional, or behavioral stimulus. Thus, the physical pain that is of psychological origin. Headaches, muscle pains, back pain, and stomach pains are some of the most common types of psychogenic pain observed in subjects.

Analgesia, or the reduction of pain perception, can be attained by many methods including directly decreasing transmission along such nociceptive pathways by using for example opiates, and inhibiting release of neurotransmitters (See U.S. Pat. No. 8,268,774, which is incorporated by reference, herein, in its entirety).

Without being limited by any particular theory or mechanism of action, it is here envisioned that binding proteins or peptides described herein are effective delivery vehicles for an agent (e.g., therapeutic and diagnostic) for treatment of pain. A pharmaceutical composition used for treatment of a subject comprises the binding protein or peptide herein; and at least one therapeutic agent.

In various embodiments, the pharmaceutical composition includes the binding protein or peptide, and a detectable agent. In various embodiments, the detectable agent comprises a detectable agent or imaging agent for analysis of the brain. For example the detectable agent comprises a fluorescent agent, a colorimetric agent, an enzymatic agent, or a radioactive agent.

Pharmaceutical Compositions

Pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art (US Patent Publication No. 20090311253 A1).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253 A1).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### VI. COMBINATION THERAPY

A binding protein provided herein also can also be administered with one or more additional medicaments or therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

The binding agent in various embodiments is administered with an agent that is a protein, a peptide, a carbohydrate, a drug, a small molecule, and a genetic material (e.g., DNA or RNA). In various embodiments, the agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The additional agent in various embodiments is a therapeutic agent. In various embodiments, the therapeutic agent comprises budenoside, epidermal growth factor, a corticosteroid, cyclosporin, sulfasalazine, an amino salicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody specific against or an agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1RI, sIL-1RII, sIL-6R, an anti-inflammatory cytokine, IL-4, IL-10, IL-11, IL-13, or TGFβ.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

### VII. DIAGNOSTICS

The disclosure herein also provides diagnostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US 2009-0311253 A1).

Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (US Patent Publication No. 2009-0311253 A1).

Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081, 7,419,821, and 7,682,833; and US Publication Nos. 20040018577, 20060160164 and 20090311253. It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### VIII. EXEMPLIFICATION

The present invention is further illustrated by the following examples which should not be construed as further limiting. The contents of figures and all references, patents and published patent applications cited throughout this application are expressly incorporated herein by reference in their entireties.

### Example 1

### Co-Stimulator X Engagement Suppressed EGFR/CD3 DVD-Ig Induced Tumor Killing

A431 carcinoma cells were incubated with human cytotoxic T-cells and peripheral blood mononuclear cells (PBMCs) in the presence of DVD-Ig molecules and/or monoclonal antibodies. A primary EGFR/CD3 DVD-Ig was applied at various concentrations (nanomolar, nM), and secondary DVD-Ig molecules and/or monoclonal antibodies were applied at a constant concentration. The amount of tumor cell lysis occurring at each concentration of EGFR/CD3 DVD-Ig and under each condition was measured and is shown in FIG. 1. These data indicate that co-stimulator X engagement suppressed EGFR/CD3 DVD-Ig induced tumor killing. It was observed that suppression of EGFR/CD3 DVD-Ig induced tumor killing was maximal when co-stimulator X is cross-linked with EGFR on the surface of the A431 cells. These data identify co-stimulator X as a therapeutic target that modulated the immune response caused by an EGFR/CD3 DVD-Ig therapeutic and that this effect was potentiated when co-stimulator X is engaged by a bispecific molecule that also binds to an antigen on the target cell surface.

### Example 2

### Co-Stimulator Y Engagement Enhances EGFR/CD3 DVD-Ig Mediated Tumor Killing

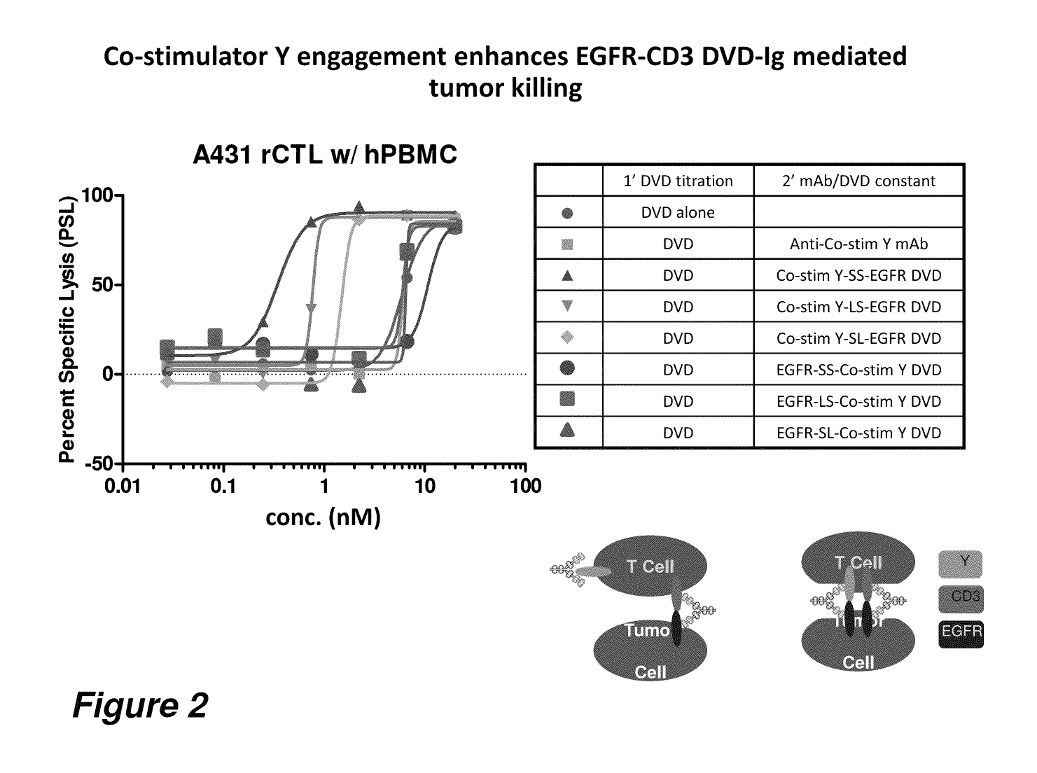

To determine whether co-stimulator Y engagement enhances EGFR/CD3 DVD-Ig mediated tumor killing, A431 carcinoma cells were incubated with human cytotoxic T-cells and PBMCs in the presence of DVD-Ig molecules with or without antibodies. Primary EGFR/CD3 DVD-Ig were administered to the T-cells/PBMCS at various concentrations, and then co-stimulator Y/EGFR DVD-Ig molecules (having different orientations), or a anti-Co stimulator Y monoclonal antibodies were applied at a constant concentration. The amount of tumor cell lysis occurring at each concentration of EGFR/CD3 DVD-Ig under each condition was measured and is shown in FIG. 2, herein. These data indicate that co-stimulator Y engagement enhanced EGFR/CD3 DVD-Ig induced tumor killing. Without being limited by any theoretical explanation, it is believed that co-stimulator Y was cross-linked with EGFR on the surface of the A431 cells. These data identify co-stimulator Y as a therapeutic target, and that a bispecific molecule that engages co-stimulator Y and also binds to an antigen on the target cell surface would be effective to mediate tumor killing.

### Example 3

### Modulation of Redirected Cytotoxicity (rCTL) Activity by CD2/EGFR DVD-Ig

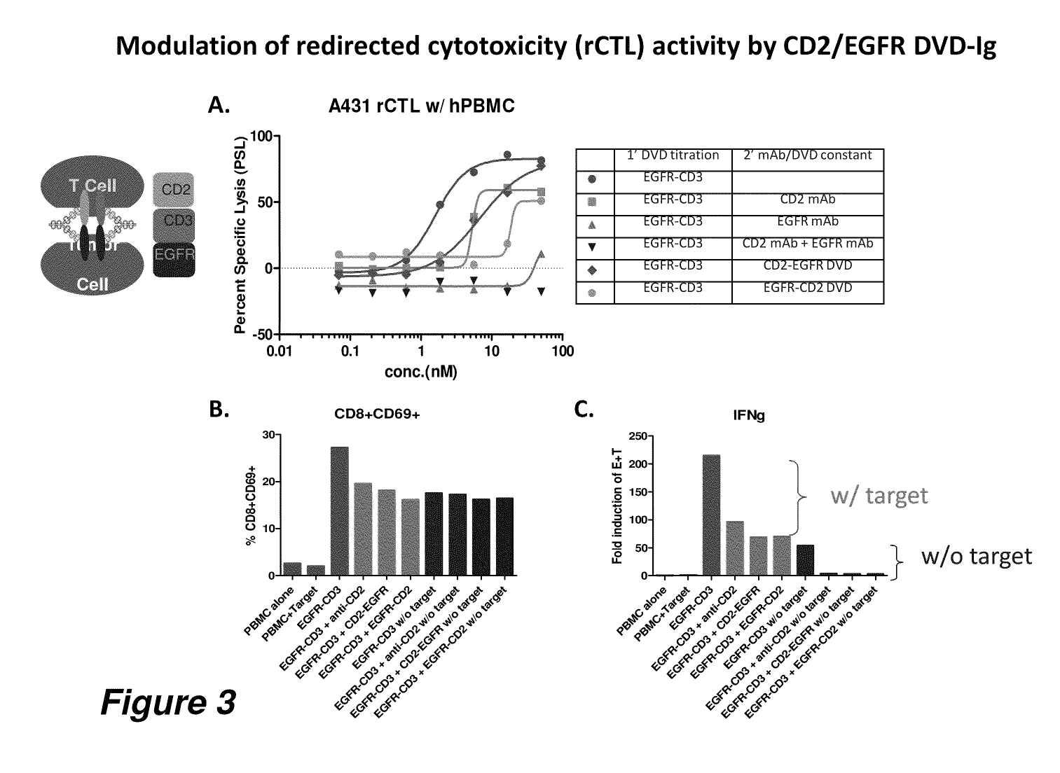

The ability of the co-stimulator CD2 to redirect CD3 mediated cytotoxicity was assessed in Examples herein (see FIG. 3). Specifically, A431 carcinoma target cells were co-cultured with effector human peripheral blood mononuclear cells (hPBMCs), and a primary EGFR/CD3 DVD-Ig was applied at various concentrations, followed by the other indicated DVD-Ig molecules and antibodies were applied at a constant concentration. Data were analyzed by calculating percent specific lysis; the cell indexes of targets in the DVD-Ig treated samples were divided by the cell indexes of control targets (no treatment). The data were graphed and an half maximal inhibitory concentration (IC₅₀) was calculated for each experimental condition. To determine whether CD2 engagement by monospecific antibodies or a bispecific DVD-Ig modulated T-cell activation and/or cytokine release, examples herein cultured T-cells were collected and their activation status was determined by FACS analysis and the cell culture media was collected and assayed for IFNγ. For FACS analysis, T-cells were stained with fluorescent labeled anti-CD4, CD8, CD25, and CD69 (eBiosciences, San Diego, Calif.) and analyzed in a FACSCanto II flow cytometer (BD Biosciences, San Jose, Calif.).

Treatment of the hPBMCs cultures with an EGFR/CD3 DVD-Ig was observed to be sufficient for T-cell activation (FIG. 3B). Co-treatment of the cultures with an anti-CD2 mAb (TS2/18.1.1) and an EGFR/CD3 DVD-Ig suppressed EGFR/CD3 DVD-Ig mediated tumor cell killing (FIG. 3A. Co-treatment of the cultures with a CD2/EGFR DVD-Ig and an EGFR/CD3 DVD-Ig induced EGFR/CD3 DVD-Ig mediated tumor cell killing maximally with markedly lower cytokine release (See FIG. 3 panels A and C). Co-treatment of the PBMC monocultures with an anti-CD2 mAb (TS2/18.1.1) and an EGFR/CD3 DVD-Ig suppressed EGFR/CD3 DVD-Ig induced cytokine release (FIG. 3C). Together, these data indicated that an anti-CD2 mAb or CD2/EGFR DVD-Ig was effective for suppressing anti-CD3 mAb or EGFR/CD3 DVD-Ig mediated non-specific cytokine release from T-cells. These data identified co-stimulator CD2 as a therapeutic target for a bispecific molecule that engaged co-stimulator CD2 and also bound to an antigen on the target cell surface.

### Example 4

### Modulation of rCTL Activity by 4-1BB/EGFR DVD-Ig

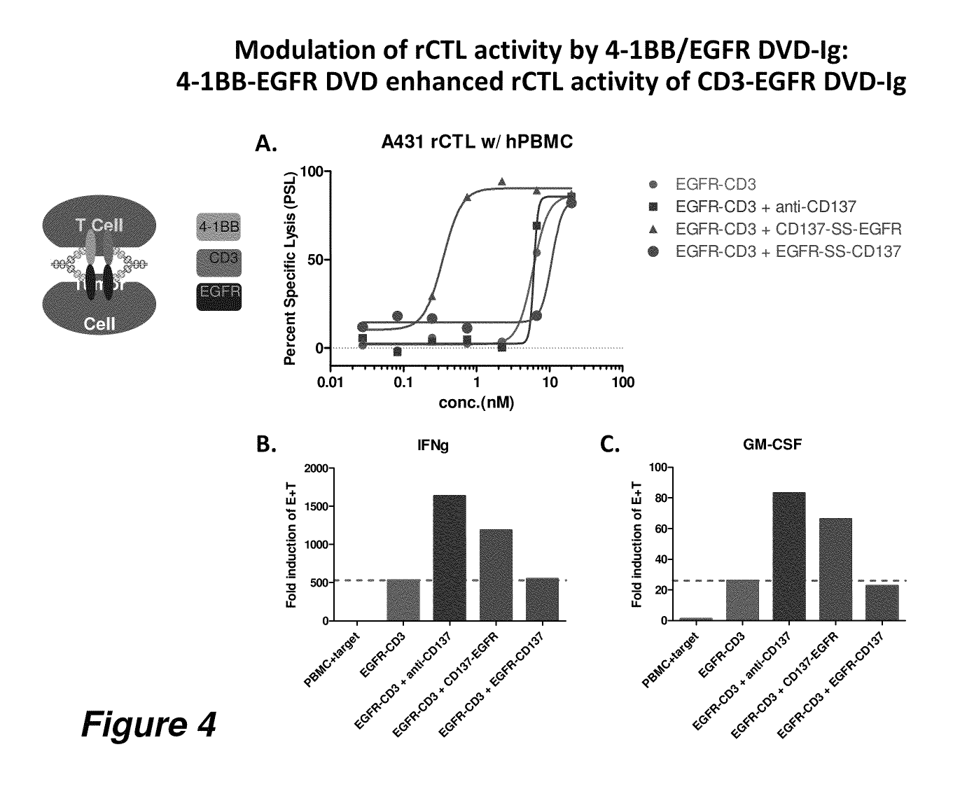

The ability of co-stimulator CD137 (4-1BB) to redirect CD3 mediated cytotoxicity was assessed with the A431/hPBMC co-culture assay described in Example 3, herein (see FIG. 4). Co-treatment with an anti-CD137 (4-1BB) mAb (BMS-663513) and an EGFR/CD3 DVD-Ig was observed not to alter EGFR/CD3 DVD-Ig mediated tumor cell killing and to induce higher cytokine release, as set forth in FIGS. 4A-4C, herein. Co-treatment with a CD137 (4-1BB)/EGFR DVD-Ig and an EGFR/CD3 DVD-Ig induced EGFR/CD3 DVD-Ig mediated tumor cell killing and enhanced cytokine release compared to EGFR/CD3 DVD-Ig alone (FIG. 4 panels A-C). Together, these data show that an anti-CD137 (4-1BB) mAb or CD137 (4-1BB)/EGFR DVD-Ig were effective to enhance anti-CD3 mAb or EGFR/CD3 DVD-Ig mediated non-specific cytokine release from T-cells. Furthermore it was observed that CD137(4-1BB)/EGFR DVD-Ig enhanced EGFR/CD3 DVD-Ig mediated tumor cell killing. These data identify co-stimulator CD137 (4-1BB) as a therapeutic target for a bispecific molecule that engage co-stimulator CD137 (4-1BB) and also bound to an antigen on the target cell surface.

### Example 5

### Initial Design and Production of Tri-Specific, Monovalent Poly-Ig

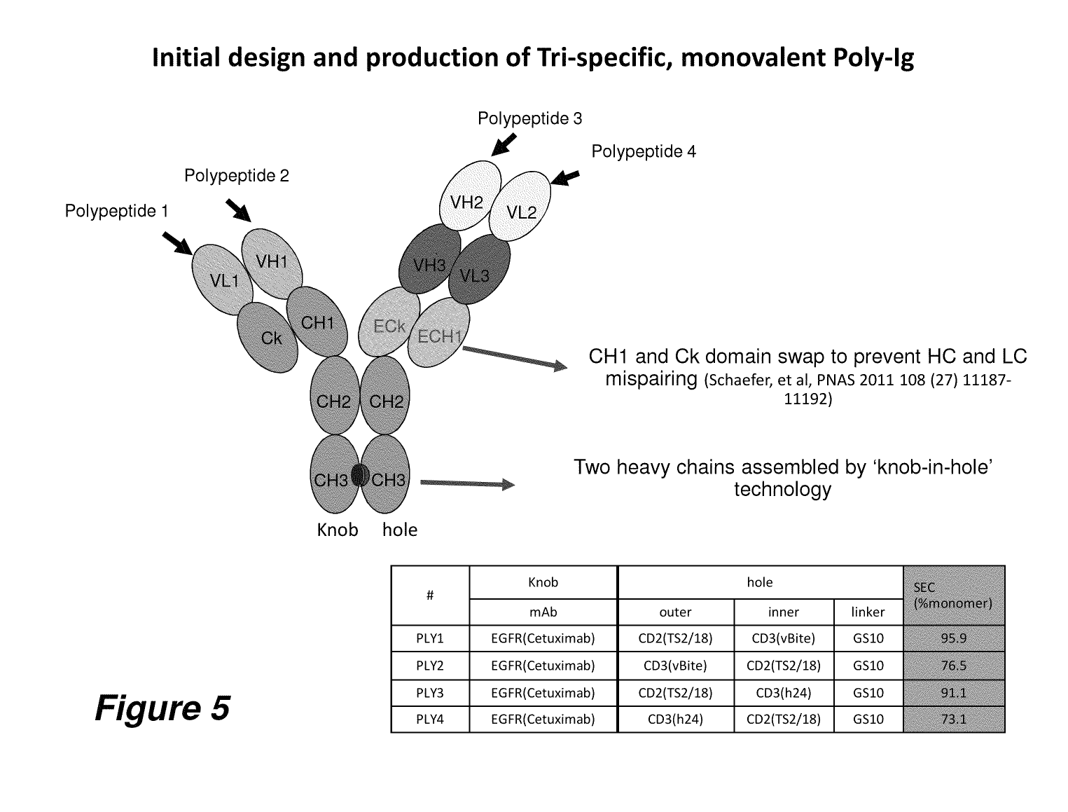

Tri-specific, monovalent proteins were designed in Examples herein using ‘knob-in-hole’ technology and a CH1/Ck domain swap to prevent the mispairing of the heavy and light chains (see FIG. 5). Plasmids encoding the heavy chains of anti-EGFR mAb or CD2/CD3 DVD-Ig operably linked to Fc domains containing mutations for ‘knob-in-hole’ were paired with a plasmid encoding a common corresponding light chain from an anti-EGFR mAb or CD2/CD3 DVD-Ig with GS10 linkers. Amino acid sequences of the Poly-Ig binding protein and antibody heavy and light chains are set forth in Table 1. Amino acid sequences of the antibody complementarity determining regions (CDRs) are set forth in Table 2. The plasmids were transfected into human embryonic kidney (HEK) 293-6E cells (American Type Culture Collection, Manassas, Va.) using polyethylenimine (Sigma, St. Louis, Mo.) for transient expression of the Tri-specific binding protein. The cell culture media was harvested six to seven days-post transient transfection and the antibodies were purified using protein A chromatography (Invitrogen, Carlsbad, Calif.) according to the manufacturer's instructions.

Size exclusion chromatography, SEC, analysis was performed to identify/confirm that the various binding proteins were tri-specific (FIG. 5). For SEC analysis, purified tri-specific binding proteins in phosphate buffered saline (PBS) were applied to a Superdex 200, 300×10 mm column (GE Healthcare, Piscataway, N.J.), and analyzed using a Model 10A HPLC instrument (Shimadzu, Columbia, Md.). All proteins were detected using UV light at 280 nm and 214 nm. The elution was isocratic at a flow rate of 0.5 mL/min.

TABLE 1
Amino acid sequences of Poly-Ig binding protein and mAb heavy and light chains

| | | | |
|---|---|---|---|
| PLY1 | SEQ ID NO: 32 | Polypeptide 1 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 33 | Polypeptide 2 | QVQLKQSGPGLVQPSQSLSITCTVSGESLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKKVEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLWCLVKGFYP |
| | | | SDIAVEWESNGQPENNYKTTPPVLDSDGSFFLYSKLTVDKSRWQQGN |
| | | | VFSCSVMHEALHNHYTQKSLSLSPGK |
| | SEQ ID NO: 34 | Polypeptide 3 | EVQLVESGGGLVMPGGSLKLSCAASGFAFSSYDMSWVRQTPEKRLEW |
| | | | VAYISGGGFTYYPDTVKGRFTLSRDNAKNTLYLQMSSLKSEDTAMYY |
| | | | CARQGANWELVYWGQGTLVTVSAGGGGSGGGGSQVQLQQSGAELARP |
| | | | GASVKMSCKASGYTFTRYTMHWVKQRPGQGLEWIGYINPSRGYTNYN |
| | | | QKFKDKATLTTDKSSSTAYMQLSSLTSEDSAVYYCARYYDDHYCLDY |
| | | | WGQGTTLTVSSASVAAPSVFIEPPSDEQLKSGTASVVCLLNNFYPRE |
| | | | AKVQWKVDNALQSGNSQESVTEQDSKDSTYSLSSTLTLSKADYEKHK |
| | | | VYACEVTHQGLSSPVTKSFNRGECEPKSCDKTHTCPPCPAPELLGGP |
| | | | SVFLEPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPI |
| | | | EKTISKAKGQPREPQVYTLPPSREEMTKNQVSLSCAVKGFYPSDIAV |
| | | | EWESNGQPENNYKTTPPVLDSDGSFFLVSKLTVDKSRWQQGNVFSCS |
| | | | VMHEALHNHYTQKSLSLSPGK |
| | SEQ ID NO: 35 | Polypeptide 4 | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRGGSGGGGSGQIVLTQSPAIMSASPGEKVTMTCR |
| | | | ASSSVSYMNWYQQKSGTSPKRWIYDTSKVASGVPYRFSGSGSGTSYS |
| | | | LTISSMEAEDAATYYCQQWSSNPLTFGSGTKLEINSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY2 | SEQ ID NO: 36 | Polypeptide 1 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 37 | Polypeptide 2 | QVQLKQSGPGLVQPSQSLSITCTVSGFSLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKKVEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLWCLVKGFYP |
| | | | SDIAVEWESNGQPENNYKTTPPVLDSDGSFFLYSKLTVDKSRWQQGN |
| | | | VFSCSVMHEALHNHYTQKSLSLSPGK |
| | SEQ ID NO: 38 | Polypeptide 3 | QVQLQQSGAELARPGASVKMSCKASGYTFTRYTMHWVKQRPGQGLEW |
| | | | IGYINPSRGYTNYNQKFKDKATLTTDKSSSTAYMQLSSLTSEDSAVY |
| | | | YCARYYDDHYCLDYWGQGTTLTVSSGGGGSGGGGSEVQLVESGGGLV |
| | | | MPGGSLKLSCAASGFAFSSYDMSWVRQTPEKRLEWVAYISGGGFTYY |
| | | | PDTVKGRFTLSRDNAKNTLYLQMSSLKSEDTAMYYCARQGANWELVY |
| | | | WGQGTLVTVSAASVAAPSVFIFPPSDEQLKSGTASVVCLLNNFYPRE |
| | | | AKVQWKVDNALQSGNSQESVTEQDSKDSTYSLSSTLTLSKADYEKHK |
| | | | VYACEVTHQGLSSPVTKSFNRGECEPKSCDKTHTCPPCPAPELLGGP |
| | | | SVFLEPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPI |
| | | | EKTISKAKGQPREPQVYTLPPSREEMTKNQVSLSCAVKGFYPSDIAV |
| | | | EWESNGQPENNYKTTPPVLDSDGSFFLVSKLTVDKSRWQQGNVFSCS |
| | | | VMHEALHNHYTQKSLSLSPGK |
| | SEQ ID NO: 39 | Polypeptide 4 | QIVLTQSPAIMSASPGEKVTMTCRASSSVSYMNWYQQKSGTSPKRWI |
| | | | YDTSKVASGVPYRFSGSGSGTSYSLTISSMEAEDAATYYCQQWSSNP |
| | | | LTFGSGTKLEINRGGSGGGGSGDIVMTQSPATLSVTPGDRVFLSCRA |
| | | | SQSISDFLHWYQQKSHESPRLLIKYASQSISGIPSRFSGSGSGSDFT |
| | | | LSINSVEPEDVGVYFCQNGHNFPPTFGGGTKLEIKSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY3 | SEQ ID NO: 40 | Polypeptide 1 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 41 | Polypeptide 2 | QVQLKQSGPGLVQPSQSLSITCTVSGFSLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKKVEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLWCLVKGFYP |
| | | | SDIAVEWESNGQPENNYKTTPPVLDSDGSFFLYSKLTVDKSRWQQGN |
| | | | VFSCSVMHEALHNHYTQKSLSLSPGK |
| | SEQ ID NO: 42 | Polypeptide 3 | QVQLQQSGAELARPGASVKMSCKASGYTFTRYTMHWVKQRPGQGLEW |
| | | | IGYINPSRGYTNYNQKFKDKATLTTDKSSSTAYMQLSSLTSEDSAVY |
| | | | YCARYYDDHYCLDYWGQGTTLTVSSGGGGSGGGGSEVQLVESGGGLV |
| | | | MPGGSLKLSCAASGFAFSSYDMSWVRQTPEKRLEWVAYISGGGFTYY |
| | | | PDTVKGRFTLSRDNAKNTLYLQMSSLKSEDTAMYYCARQGANWELVY |
| | | | WGQGTLVTVSAASVAAPSVFIFPPSDEQLKSGTASVVCLLNNFYPRE |
| | | | AKVQWKVDNALQSGNSQESVTEQDSKDSTYSLSSTLTLSKADYEKHK |
| | | | VYACEVTHQGLSSPVTKSFNRGECEPKSCDKTHTCPPCPAPELLGGP |
| | | | SVFLEPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPI |
| | | | EKTISKAKGQPREPQVYTLPPSREEMTKNQVSLSCAVKGFYPSDIAV |
| | | | EWESNGQPENNYKTTPPVLDSDGSFFLVSKLTVDKSRWQQGNVFSCS |
| | | | VMHEALHNHYTQKSLSLSPGK |
| | SEQ ID NO: 43 | Polypeptide 4 | QIVLTQSPAIMSASPGEKVTMTCRASSSVSYMNWYQQKSGTSPKRWI |
| | | | YDTSKVASGVPYRFSGSGSGTSYSLTISSMEAEDAATYYCQQWSSNP |
| | | | LTFGSGTKLEINRGGSGGGGSGDIVMTQSPATLSVTPGDRVFLSCRA |
| | | | SQSISDFLHWYQQKSHESPRLLIKYASQSISGIPSRFSGSGSGSDFT |
| | | | LSINSVEPEDVGVYFCQNGHNFPPTFGGGTKLEIKSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY4 | SEQ ID NO: 44 | Polypeptide 1 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 45 | Polypeptide 2 | QVQLKQSGPGLVQPSQSLSITCTVSGFSLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKKVEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLWCLVKGFYP |
| | | | SDIAVEWESNGQPENNYKTTPPVLDSDGSFFLYSKLTVDKSRWQQGN |
| | | | VFSCSVMHEALHNHYTQKSLSLSPGK |
| | SEQ ID NO: 46 | Polypeptide 3 | EVQLVESGGGLVQPGGSLRLSCAASGFTFSTYAINWVRQAPGKGLEW |
| | | | VGRIRSKYNNYATYYADSVKDRFTISRDDSKNTAYLQMNSLRAEDTA |
| | | | VYYCTRHGNFGNSYVSWFAYWGQGTLVTVSSGGGGSGGGGSEVQLVE |
| | | | SGGGLVMPGGSLKLSCAASGFAFSSYDMSWVRQTPEKRLEWVAYISG |
| | | | GGFTYYPDTVKGRFTLSRDNAKNTLYLQMSSLKSEDTAMYYCARQGA |
| | | | NWELVYWGQGTLVTVSAASVAAPSVFIFPPSDEQLKSGTASVVCLLN |
| | | | NFYPREAKVQWKVDNALQSGNSQESVTEQDSKDSTYSLSSTLTLSKA |
| | | | DYEKHKVYACEVTHQGLSSPVTKSFNRGECEPKSCDKTHTCPPCPAP |
| | | | ELLGGPSVFLEPPKPKDTLMISRTPEVTCVVVDVSHEDPEVKFNWYV |
| | | | DGVEVHNAKTKPREEQYNSTYRVVSVLTVLHQDWLNGKEYKCKVSNK |
| | | | ALPAPIEKTISKAKGQPREPQVYTLPPSREEMTKNQVSLSCAVKGFY |
| | | | PSDIAVEWESNGQPENNYKTTPPVLDSDGSFFLVSKLTVDKSRWQQG |
| | | | NVFSCSVMHEALHNHYTQKSLSLSPGK |
| | SEQ ID NO: 47 | Polypeptide 4 | DAQVTQSPSSLSASVGDRVTITCRSSTGAVTTSNYANWVQEKPGKLF |
| | | | KGLIGGTNKRAPGVPSRFSGSGSGTDATLTISSLQPEDFATYFCALW |
| | | | YSNLWVFGGGTKVEIKRGGSGGGGSGDIVMTQSPATLSVTPGDRVFL |
| | | | SCRASQSISDFLHWYQQKSHESPRLLIKYASQSISGIPSRFSGSGSG |
| | | | SDFTLSINSVEPEDVGVYFCQNGHNFPPTFGGGTKLEIKSSASTKGP |
| | | | SVFPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKKV |
| PLY5 | SEQ ID NO: 48 | Polypeptide 1 | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRTVAAPSVFIEPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 49 | Polypeptide 2 | EVQLVESGGGLVMPGGSLKLSCAASGFAFSSYDMSWVRQTPEKRLEW |
| | | | VAYISGGGFTYYPDTVKGRFTLSRDNAKNTLYLQMSSLKSEDTAMYY |
| | | | CARQGANWELVYWGQGTLVTVSAASTKGPSVFPLAPSSKSTSGGTAA |
| | | | LGCLVKDYFPEPVTVSWNSGALTSGVHTFPAVLQSSGLYSLSSVVTV |
| | | | PSSSLGTQTYICNVNHKPSNTKVDKKVEPKSCDKTHTCPPCPAPELL |
| | | | GGPSVFLEPPKPKDTLMISRTPEVTCVVVDVSHEDPEVKFNWYVDGV |
| | | | EVHNAKTKPREEQYNSTYRVVSVLTVLHQDWLNGKEYKCKVSNKALP |
| | | | APIEKTISKAKGQPREPQVYTLPPSREEMTKNQVSLWCLVKGFYPSD |
| | | | IAVEWESNGQPENNYKT |
| | SEQ ID NO: 50 | Polypeptide 3 | QVQLQESGPGLVKPSQTLSLTCTVSGYSISSDFAWNWIRQPPGKGLE |
| | | | WMGYISYSGNTRYQPSLKSRITISRDTSKNQFFLKLNSVTAADTATY |
| | | | YCVTAGRGFPYWGQGTLVTVSSGGGGSGGGGSQVQLQQSGAELARPG |
| | | | ASVKMSCKASGYTFTRYTMHWVKQRPGQGLEWIGYINPSRGYTNYNQ |
| | | | KFKDKATLTTDKSSSTAYMQLSSLTSEDSAVYYCARYYDDHYCLDYW |
| | | | GQGTTLTVSSASVAAPSVFIFPPSDEQLKSGTASVVCLLNNFYPREA |
| | | | KVQWKVDNALQSGNSQESVTEQDSKDSTYSLSSTLTLSKADYEKHKV |
| | | | YACEVTHQGLSSPVTKSFNRGECEPKSCDKTHTCPPCPAPELLGGPS |
| | | | VFLEPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIE |
| | | | KTISKAKGQPREPQVYTLPPSREEMTKNQVSLSCAVKGFYPSDIAVE |
| | | | WESNGQPENNYKTTPPVLDSDGSFFLVSKLTVDKSRWQQGNVFSCSV |
| | | | MHEALHNHYTQKSLSLSPGK |
| | SEQ ID NO: 51 | Polypeptide 4 | DIQMTQSPSSMSVSVGDRVTITCHSSQDINSNIGWLQQKPGKSFKGL |
| | | | IYHGTNLDDGVPSRFSGSGSGTDYTLTISSLQPEDFATYYCVQYAQF |
| | | | PWTFGGGTKLEIKRGGSGGGGSGQIVLTQSPAIMSASPGEKVTMTCR |
| | | | ASSSVSYMNWYQQKSGTSPKRWIYDTSKVASGVPYRFSGSGSGTSYS |
| | | | LTISSMEAEDAATYYCQQWSSNPLTFGSGTKLEINSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY6 | SEQ ID NO: 52 | Polypeptide 1 | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRTVAAPSVFIEPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 53 | Polypeptide 2 | EVQLVESGGGLVMPGGSLKLSCAASGFAFSSYDMSWVRQTPEKRLEW |
| | | | VAYISGGGFTYYPDTVKGRFTLSRDNAKNTLYLQMSSLKSEDTAMYY |
| | | | CARQGANWELVYWGQGTLVTVSAASTKGPSVFPLAPSSKSTSGGTAA |
| | | | LGCLVKDYFPEPVTVSWNSGALTSGVHTFPAVLQSSGLYSLSSVVTV |
| | | | PSSSLGTQTYICNVNHKPSNTKVDKKVEPKSCDKTHTCPPCPAPELL |
| | | | GGPSVFLEPPKPKDTLMISRTPEVTCVVVDVSHEDPEVKFNWYVDGV |
| | | | EVHNAKTKPREEQYNSTYRVVSVLTVLHQDWLNGKEYKCKVSNKALP |
| | | | APIEKTISKAKGQPREPQVYTLPPSREEMTKNQVSLWCLVKGFYPSD |
| | | | IAVEWESNGQPENNYKT |
| | SEQ ID NO: 54 | Polypeptide 3 | QVQLQESGPGLVKPSQTLSLTCTVSGYSISSDFAWNWIRQPPGKGLE |
| | | | WMGYISYSGNTRYQPSLKSRITISRDTSKNQFFLKLNSVTAADTATY |
| | | | YCVTAGRGFPYWGQGTLVTVSSGGGGSGGGGSEVQLVESGGGLVQPG |
| | | | GSLRLSCAASGFTFSTYAMNWVRQAPGKGLEWVGRIRSKYNNYATYY |
| | | | ADSVKDRFTISRDDSKNTAYLQMNSLRAEDTAVYYCTRHGNFGNSYV |
| | | | SWFAYWGQGTLVTVSSASVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGECEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLSCAVKGFYP |
| | | | SDIAVEWESNGQPENNYKTTPPVLDSDGSFFLVSKLTVDKSRWQQGN |
| | | | VFSCSVMHEALHNHYTQKSLSLSPGK |
| | SEQ ID NO: 55 | Polypeptide 4 | DIQMTQSPSSMSVSVGDRVTITCHSSQDINSNIGWLQQKPGKSFKGL |
| | | | IYHGTNLDDGVPSRFSGSGSGTDYTLTISSLQPEDFATYYCVQYAQF |
| | | | PWTFGGGTKLEIKRGGSGGGGSGDAQVTQSPSSLSASVGDRVTITCR |
| | | | SSTGAVTTSNYANWVQEKPGKLFKGLIGGTNKRAPGVPSRFSGSGSG |
| | | | TDATLTISSLQPEDFATYFCALWYSNLWVFGGGTKVEIKSSASTKGP |
| | | | SVFPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKKV |
| PLY7 | SEQ ID NO: 56 | Polypeptide 1 | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 57 | Polypeptide 2 | EVQLVESGGGLVMPGGSLKLSCAASGFAFSSYDMSWVRQTPEKRLEW |
| | | | VAYISGGGFTYYPDTVKGRFTLSRDNAKNTLYLQMSSLKSEDTAMYY |
| | | | CARQGANWELVYWGQGTLVTVSAASTKGPSVFPLAPSSKSTSGGTAA |
| | | | LGCLVKDYFPEPVTVSWNSGALTSGVHTFPAVLQSSGLYSLSSVVTV |
| | | | PSSSLGTQTYICNVNHKPSNTKVDKKVEPKSCDKTHTCPPCPAPELL |
| | | | GGPSVFLEPPKPKDTLMISRTPEVTCVVVDVSHEDPEVKFNWYVDGV |
| | | | EVHNAKTKPREEQYNSTYRVVSVLTVLHQDWLNGKEYKCKVSNKALP |
| | | | APIEKTISKAKGQPREPQVYTLPPSREEMTKNQVSLWCLVKGFYPSD |
| | | | IAVEWESNGQPENNYKT |
| | SEQ ID NO: 58 | Polypeptide 3 | QVQLKQSGPGLVQPSQSLSITCTVSGFSLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAGGGGSGGGGSQVQLQQSGAELA |
| | | | RPGASVKMSCKASGYTFTRYTMHWVKQRPGQGLEWIGYINPSRGYTN |
| | | | YNQKFKDKATLTTDKSSSTAYMQLSSLTSEDSAVYYCARYYDDHYCL |
| | | | DYWGQGTTLTVSSASVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGECEPKSCDKTHTCPPCPAPELLG |
| | | | GPSVFLEPPKPKDTLMISRTPEVTCVVVDVSHEDPEVKFNWYVDGVE |
| | | | VHNAKTKPREEQYNSTYRVVSVLTVLHQDWLNGKEYKCKVSNKALPA |
| | | | PIEKTISKAKGQPREPQVYTLPPSREEMTKNQVSLSCAVKGFYPSDI |
| | | | AVEWESNGQPENNYKTTPPVLDSDGSFFLVSKLTVDKSRWQQGNVFS |
| | | | CSVMHEALHNHYTQKSLSLSPGK |
| | SEQ ID NO: 59 | Polypeptide 4 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRGGSGGGGSGQIVLTQSPAIMSASPGEKVTMTCR |
| | | | ASSSVSYMNWYQQKSGTSPKRWIYDTSKVASGVPYRFSGSGSGTSYS |
| | | | LTISSMEAEDAATYYCQQWSSNPLTFGSGTKLEINSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY8 | SEQ ID NO: 60 | Polypeptide 1 | EIVLTQSPATLSLSPGERATLSCRASQSVSSYLAWYQQKPGQAPRLL |
| | | | IYDASNRATGIPARFSGSGSGTDFTLTISSLEPEDFAVYYCQQRSNW |
| | | | PPALTEGGGTKVEIKRTVAAPSVFIFPPSDEQLKSGTASVVCLLNNF |
| | | | YPREAKVQWKVDNALQSGNSQESVTEQDSKDSTYSLSSTLTLSKADY |
| | | | EKHKVYACEVTHQGLSSPVTKSFNRGEC |
| | SEQ ID NO: 61 | Polypeptide 2 | QVQLQQWGAGLLKPSETLSLTCAVYGGSFSGYYWSWIRQSPEKGLEW |
| | | | IGEINHGGYVTYNPSLESRVTISVDTSKNQFSLKLSSVTAADTAVYY |
| | | | CARDYGPGNYDWYFDLWGRGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCPA |
| | | | PEAAGGPSVFLEPPKPKDTLMISRTPEVTCVVVDVSHEDPEVKFNWY |
| | | | VDGVEVHNAKTKPREEQYNSTYRVVSVLTVLHQDWLNGKEYKCKVSN |
| | | | KALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPGK |
| | SEQ ID NO: 62 | Polypeptide 3 | QVQLQESGPGLVKPSQTLSLICTVSGYSISSDFAWNWIRQPPGKGLE |
| | | | WMGYISYSGNTRYQPSLKSRITISRDTSKNQFFLKLNSVTAADTATY |
| | | | YCVTAGRGFPYWGQGTLVTVSSGGGGSGGGGSQVQLQQSGAELARPG |
| | | | ASVKMSCKASGYTFTRYTMHWVKQRPGQGLEWIGYINPSRGYTNYNQ |
| | | | KFKDKATLTTDKSSSTAYMQLSSLTSEDSAVYYCARYYDDHYCLDYW |
| | | | GQGTTLTVSSASVAAPSVFIFPPSDEQLKSGTASVVCLLNNFYPREA |
| | | | KVQWKVDNALQSGNSQESVTEQDSKDSTYSLSSTLTLSKADYEKHKV |
| | | | YACEVTHQGLSSPVTKSFNRGECEPKSCDKTHTCPPCPAPELLGGPS |
| | | | VFLEPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIE |
| | | | KTISKAKGQPREPQVYTLPPSREEMTKNQVSLSCAVKGFYPSDIAVE |
| | | | WESNGQPENNYKTTPPVLDSDGSFFLVSKLTVDKSRWQQGNVFSCSV |
| | | | MHEALHNHYTQKSLSLSPGK |
| | SEQ ID NO: 63 | Polypeptide 4 | DIQMTQSPSSMSVSVGDRVTITCHSSQDINSNIGWLQQKPGKSFKGL |
| | | | IYHGTNLDDGVPSRFSGSGSGTDYTLTISSLQPEDFATYYCVQYAQF |
| | | | PWTEGGGTKLEIKRGGSGGGGSGQIVLTQSPAIMSASPGEKVTMTCR |
| | | | ASSSVSYMNWYQQKSGTSPKRWIYDTSKVASGVPYRFSGSGSGTSYS |
| | | | LTISSMEAEDAATYYCQQWSSNPLTEGSGTKLEINSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY9 | SEQ ID NO: 64 | Polypeptide 1 | EIVLTQSPATLSLSPGERATLSCRASQSVSSYLAWYQQKPGQAPRLL |
| | | | IYDASNRATGIPARFSGSGSGTDFTLTISSLEPEDFAVYYCQQRSNW |
| | | | PPALTEGGGTKVEIKRTVAAPSVFIFPPSDEQLKSGTASVVCLLNNF |
| | | | YPREAKVQWKVDNALQSGNSQESVTEQDSKDSTYSLSSTLTLSKADY |
| | | | EKHKVYACEVTHQGLSSPVTKSFNRGEC |
| | SEQ ID NO: 65 | Polypeptide 2 | QVQLQQWGAGLLKPSETLSLTCAVYGGSFSGYYWSWIRQSPEKGLEW |
| | | | IGEINHGGYVTYNPSLESRVTISVDTSKNQFSLKLSSVTAADTAVYY |
| | | | CARDYGPGNYDWYFDLWGRGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCPA |
| | | | PEAAGGPSVFLEPPKPKDTLMISRTPEVTCVVVDVSHEDPEVKFNWY |
| | | | VDGVEVHNAKTKPREEQYNSTYRVVSVLTVLHQDWLNGKEYKCKVSN |
| | | | KALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPGK |
| | SEQ ID NO: 66 | Polypeptide 3 | QVQLQESGPGLVKPSQTLSLTCTVSGYSISSDFAWNWIRQPPGKGLE |
| | | | WMGYISYSGNTRYQPSLKSRITISRDTSKNQFFLKLNSVTAADTATY |
| | | | YCVTAGRGFPYWGQGTLVTVSSGGGGSGGGGSEVQLVESGGGLVQPG |
| | | | GSLRLSCAASGFTFSTYAMNWVRQAPGKGLEWVGRIRSKYNNYATYY |
| | | | ADSVKDRFTISRDDSKNTAYLQMNSLRAEDTAVYYCTRHGNFGNSYV |
| | | | SWFAYWGQGTLVTVSSASVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGECEPKSCDKTHTCPPCPAPE |
| | | | LLGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLSCAVKGFYP |
| | | | SDIAVEWESNGQPENNYKTTPPVLDSDGSFFLVSKLTVDKSRWQQGN |
| | | | VFSCSVMHEALHNHYTQKSLSLSPGK |
| | SEQ ID NO: 67 | Polypeptide 4 | DIQMTQSPSSMSVSVGDRVTITCHSSQDINSNIGWLQQKPGKSFKGL |
| | | | IYHGTNLDDGVPSRFSGSGSGTDYTLTISSLQPEDFATYYCVQYAQF |
| | | | PWTFGGGTKLEIKRGGSGGGGSGDAQVTQSPSSLSASVGDRVTITCR |
| | | | SSTGAVTTSNYANWVQEKPGKLFKGLIGGTNKRAPGVPSRFSGSGSG |
| | | | TDATLTISSLQPEDFATYFCALWYSNLWVFGGGTKVEIKSSASTKGP |
| | | | SVFPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKKV |
| PLY10 | SEQ ID NO: 68 | Polypeptide 1 | EIVLTQSPATLSLSPGERATLSCRASQSVSSYLAWYQQKPGQAPRLL |
| | | | IYDASNRATGIPARFSGSGSGTDFTLTISSLEPEDFAVYYCQQRSNW |
| | | | PPALTFGGGTKVEIKRTVAAPSVFIFPPSDEQLKSGTASVVCLLNNF |
| | | | YPREAKVQWKVDNALQSGNSQESVTEQDSKDSTYSLSSTLTLSKADY |
| | | | EKHKVYACEVTHQGLSSPVTKSFNRGEC |
| | SEQ ID NO: 69 | Polypeptide 2 | QVQLQQWGAGLLKPSETLSLTCAVYGGSFSGYYWSWIRQSPEKGLEW |
| | | | IGEINHGGYVTYNPSLESRVTISVDTSKNQFSLKLSSVTAADTAVYY |
| | | | CARDYGPGNYDWYFDLWGRGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCPA |
| | | | PEAAGGPSVFLEPPKPKDTLMISRTPEVTCVVVDVSHEDPEVKFNWY |
| | | | VDGVEVHNAKTKPREEQYNSTYRVVSVLTVLHQDWLNGKEYKCKVSN |
| | | | KALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPGK |
| | SEQ ID NO: 70 | Polypeptide 1 | QVQLKQSGPGLVQPSQSLSITCTVSGFSLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAGGGGSGGGGSQVQLQQSGAELA |
| | | | RPGASVKMSCKASGYTFTRYTMHWVKQRPGQGLEWIGYINPSRGYTN |
| | | | YNQKFKDKATLTTDKSSSTAYMQLSSLTSEDSAVYYCARYYDDHYCL |
| | | | DYWGQGTTLTVSSASVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGECEPKSCDKTHTCPPCPAPELLG |
| | | | GPSVFLEPPKPKDTLMISRTPEVTCVVVDVSHEDPEVKFNWYVDGVE |
| | | | VHNAKTKPREEQYNSTYRVVSVLTVLHQDWLNGKEYKCKVSNKALPA |
| | | | PIEKTISKAKGQPREPQVYTLPPSREEMTKNQVSLSCAVKGFYPSDI |
| | | | AVEWESNGQPENNYKTTPPVLDSDGSFFLVSKLTVDKSRWQQGNVFS |
| | | | CSVMHEALHNHYTQKSLSLSPGK |
| | SEQ ID NO: 71 | Polypeptide 2 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRGGSGGGGSGQIVLTQSPAIMSASPGEKVTMTCR |
| | | | ASSSVSYMNWYQQKSGTSPKRWIYDTSKVASGVPYRFSGSGSGTSYS |
| | | | LTISSMEAEDAATYYCQQWSSNPLTFGSGTKLEINSSASTKGPSVFP |
| | | | LAPSSKSTSGGTAALGCLVKDYFPEPVTVSWNSGALTSGVHTFPAVL |
| | | | QSSGLYSLSSVVTVPSSSLGTQTYICNVNHKPSNTKVDKKV |
| PLY11 | SEQ ID NO: 72 | Polypeptide 1 | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| | SEQ ID NO: 73 | Polypeptide 2 | EVQLVESGGGLVMPGGSLKLSCAASGFAFSSYDMSWVRQTPEKRLEW |
| | | | VAYISGGGFTYYPDTVKGRFTLSRDNAKNTLYLQMSSLKSEDTAMYY |
| | | | CARQGANWELVYWGQGTLVTVSAASTKGPSVFPLAPSSKSTSGGTAA |
| | | | LGCLVKDYFPEPVTVSWNSGALTSGVHTFPAVLQSSGLYSLSSVVTV |
| | | | PSSSLGTQTYICNVNHKPSNTKVDKKVEPKSCDKTHTCPPCPAPELL |
| | | | GGPSVFLEPPKPKDTLMISRTPEVTCVVVDVSHEDPEVKFNWYVDGV |
| | | | EVHNAKTKPREEQYNSTYRVVSVLTVLHQDWLNGKEYKCKVSNKALP |
| | | | APIEKTISKAKGQPREPQVYTLPPSREEMTKNQVSLWCLVKGFYPSD |
| | | | IAVEWESNGQPENNYKTTPPVLDSDGSFFLYSKLTVDKSRWQQGNVF |
| | | | SCSVMHEALHNHYTQKSLSLSPGK |
| | SEQ ID NO: 74 | Polypeptide 3 | QVQLKQSGPGLVQPSQSLSITCTVSGESLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPQVQLQQSGA |
| | | | ELARPGASVKMSCKASGYTFTRYTMHWVKQRPGQGLEWIGYINPSRG |
| | | | YTNYNQKFKDKATLTTDKSSSTAYMQLSSLTSEDSAVYYCARYYDDH |
| | | | YCLDYWGQGTTLTVSSASTKGPSVFPLAPSSKSTSGGTAALGCLVKD |
| | | | YFPEPVTVSWNSGALTSGVHTFPAVLQSSGLYSLSSVVTVPSSSLGT |
| | | | QTYICNVNHKPSNTKVDKKVEPKSCDKTHTCPPCPAPELLGGPSVFL |
| | | | FPPKPKDTLMISRTPEVTCVVVDVSHEDPEVKFNWYVDGVEVHNAKT |
| | | | KPREEQYNSTYRVVSVLTVLHQDWLNGKEYKCKVSNKALPAPIEKTI |
| | | | SKAKGQPREPQVYTLPPSREEMTKNQVSLSCAVKGFYPSDIAVEWES |
| | | | NGQPENNYKTTPPVLDSDGSFFLVSKLTVDKSRWQQGNVFSCSVMHE |
| | | | ALHNHYTQKSLSLSPGK |
| | SEQ ID NO: 75 | Polypeptide 4 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPQIVLTQSPAIMSASPGEKVTM |
| | | | TCRASSSVSYMNWYQQKSGTSPKRWIYDTSKVASGVPYRFSGSGSGT |
| | | | SYSLTISSMEAEDAATYYCQQWSSNPLTFGSGTKLEINRTVAAPSVF |
| | | | IFPPSDEQLKSGTASVVCLLNNFYPREAKVQWKVDNALQSGNSQESV |
| | | | TEQDSKDSTYSLSSTLTLSKADYEKHKVYACEVTHQGLSSPVTKSFN |
| | | | RGEC |
| PLY12 | SEQ ID NO: 76 | Polypeptide 1 | EIVLTQSPATLSLSPGERATLSCRASQSVSSYLAWYQQKPGQAPRLL |
| | | | IYDASNRATGIPARFSGSGSGTDFTLTISSLEPEDFAVYYCQQRSNW |
| | | | PPALTFGGGTKVEIKRTVAAPSVFIFPPSDEQLKSGTASVVCLLNNF |
| | | | YPREAKVQWKVDNALQSGNSQESVTEQDSKDSTYSLSSTLTLSKADY |
| | | | EKHKVYACEVTHQGLSSPVTKSFNRGEC |
| | SEQ ID NO: 77 | Polypeptide 2 | QVQLQQWGAGLLKPSETLSLTCAVYGGSFSGYYWSWIRQSPEKGLEW |
| | | | IGEINHGGYVTYNPSLESRVTISVDTSKNQFSLKLSSVTAADTAVYY |
| | | | CARDYGPGNYDWYFDLWGRGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCPA |
| | | | PELLGGPSVFLEPPKPKDTLMISRTPEVTCVVVDVSHEDPEVKFNWY |
| | | | VDGVEVHNAKTKPREEQYNSTYRVVSVLTVLHQDWLNGKEYKCKVSN |
| | | | KALPAPIEKTISKAKGQPREPQVYTLPPSREEMTKNQVSLWCLVKGF |
| | | | YPSDIAVEWESNGQPENNYKTTPPVLDSDGSFFLYSKLTVDKSRWQQ |
| | | | GNVFSCSVMHEALHNHYTQKSLSLSPGK |
| | SEQ ID NO: 78 | Polypeptide 3 | QVQLKQSGPGLVQPSQSLSITCTVSGESLTNYGVHWVRQSPGKGLEW |
| | | | LGVIWSGGNTDYNTPFTSRLSINKDNSKSQVFFKMNSLQSNDTAIYY |
| | | | CARALTYYDYEFAYWGQGTLVTVSAASTKGPSVFPLAPQVQLQQSGA |
| | | | ELARPGASVKMSCKASGYTFTRYTMHWVKQRPGQGLEWIGYINPSRG |
| | | | YTNYNQKFKDKATLTTDKSSSTAYMQLSSLTSEDSAVYYCARYYDDH |
| | | | YCLDYWGQGTTLTVSSASTKGPSVFPLAPSSKSTSGGTAALGCLVKD |
| | | | YFPEPVTVSWNSGALTSGVHTFPAVLQSSGLYSLSSVVTVPSSSLGT |
| | | | QTYICNVNHKPSNTKVDKKVEPKSCDKTHTCPPCPAPELLGGPSVFL |
| | | | FPPKPKDTLMISRTPEVTCVVVDVSHEDPEVKFNWYVDGVEVHNAKT |
| | | | KPREEQYNSTYRVVSVLTVLHQDWLNGKEYKCKVSNKALPAPIEKTI |
| | | | SKAKGQPREPQVYTLPPSREEMTKNQVSLSCAVKGFYPSDIAVEWES |
| | | | NGQPENNYKTTPPVLDSDGSFFLVSKLTVDKSRWQQGNVFSCSVMHE |
| | | | ALHNHYTQKSLSLSPGK |
| | SEQ ID NO: 79 | Polypeptide 4 | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPQIVLTQSPAIMSASPGEKVTM |
| | | | TCRASSSVSYMNWYQQKSGTSPKRWIYDTSKVASGVPYRFSGSGSGT |
| | | | SYSLTISSMEAEDAATYYCQQWSSNPLTFGSGTKLEINRTVAAPSVF |
| | | | IFPPSDEQLKSGTASVVCLLNNEYPREAKVQWKVDNALQSGNSQESV |
| | | | TEQDSKDSTYSLSSTLTLSKADYEKHKVYACEVTHQGLSSPVTKSFN |
| | | | RGEC |
| anti-CD2 | SEQ ID NO: 80 | Heavy chain | EVQLVESGGGLVMPGGSLKLSCAASGFAFXSYDMSWVRQTPEKRLEW |
| mAb(TS2/ | | | VAYISGGGFTYYPDTVKGRFTLSRDNAKNTLYLQMSSLKSEDTAMYY |
| 18.1.1) | | | CARQGANWELVYWGQGTLVTVSAASTKGPSVFPLAPSSKSTSGGTAA |
| | | | LGCLVKDYFPEPVTVSWNSGALTSGVHTFPAVLQSSGLYSLSSVVTV |
| | | | PSSSLGTQTYICNVNHKPSNTKVDKKVEPKSCDKTHTCPPCPAPEAA |
| | | | GGPSVFLEPPKPKDTLMISRTPEVTCVVVDVSHEDPEVKFNWYVDGV |
| | | | EVHNAKTKPREEQYNSTYRVVSVLTVLHQDWLNGKEYKCKVSNKALP |
| | | | APIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVF |
| | | | SCSVMHEALHNHYTQKSLSLSPGK |
| | SEQ ID NO: 81 | Heavy chain | DIVMTQSPATLSVTPGDRVFLSCRASQSISDFLHWYQQKSHESPRLL |
| | | | IKYASQSISGIPSRFSGSGSGSDFTLSINSVEPEDVGVYFCQNGHNF |
| | | | PPTFGGGTKLEIKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| anti-CD137 | SEQ ID NO: 82 | Heavy chain | QVQLQQWGAGLLKPSETLSLTCAVYGGSFSGYYWSWIRQSPEKGLEW |
| | | | IGEINHGGYVTYNPSLESRVTISVDTSKNQFSLKLSSVTAADTAVYY |
| | | | CARDYGPGNYDWYFDLWGRGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCPA |
| | | | PEAAGGPSVFLEPPKPKDTLMISRTPEVTCVVVDVSHEDPEVKFNWY |
| | | | VDGVEVHNAKTKPREEQYNSTYRVVSVLTVLHQDWLNGKEYKCKVSN |
| | | | KALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPGK |
| | SEQ ID NO: 83 | Light chain | EIVLTQSPATLSLSPGERATLSCRASQSVSSYLAWYQQKPGQAPRLL |
| | | | IYDASNRATGIPARFSGSGSGTDFTLTISSLEPEDFAVYYCQQRSNW |
| | | | PPALTFGGGTKVEIKRTVAAPSVFIFPPSDEQLKSGTASVVCLLNNF |
| | | | YPREAKVQWKVDNALQSGNSQESVTEQDSKDSTYSLSSTLTLSKADY |
| | | | EKHKVYACEVTHQGLSSPVTKSFNRGEC |
| anti- | SEQ ID NO: 84 | Heavy chain | QVQLKQSGPGLVQPSQSLSITCTVSGESLTNYGVHWVRQSPGKGLEW |
| EGFR | | | LGVIWSGGNTDYNTPFTSRLSINKDNSKSQVFFKMNSLQSNDTAIYY |
| (cetuximab) | | | CARALTYYDYEFAYWGQGTLVTVSAASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKKVEPKSCDKTHTCPPCPAPE |
| | | | AAGGPSVFLEPPKPKDTLMISRTPEVTCVVVDVSHEDPEVKFNWYVD |
| | | | GVEVHNAKTKPREEQYNSTYRVVSVLTVLHQDWLNGKEYKCKVSNKA |
| | | | LPAPIEKTISKAKGQPREPQVYTLPPSREEMTKNQVSLTCLVKGFYP |
| | | | SDIAVEWESNGQPENNYKTTPPVLDSDGSFFLYSKLTVDKSRWQQGN |
| | | | VFSCSVMHEALHNHYTQKSLSLSPGK |
| | SEQ ID NO: 85 | Light chain | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKRTVAAPSVFIFPPSDEQLKSGTASVVCLLNNFYP |
| | | | REAKVQWKVDNALQSGNSQESVTEQDSKDSTYSLSSTLTLSKADYEK |
| | | | HKVYACEVTHQGLSSPVTKSFNRGEC |
| Anti-CD3 | SEQ ID NO: 86 | VH | QVQLQQSGAELARPGASVKMSCKASGYTFTRYTMHWVKQRPGQGLEW |
| | | | IGYINPSRGYTNYNQKFKDKATLTTDKSSSTAYMQLSSLTSEDSAVY |
| | | | YCARYYDDHYCLDYWGQGTTLTVSS |
| | SEQ ID NO: 87 | VL | QIVLTQSPAIMSASPGEKVTMTCRASSSVSYMNWYQQKSGTSPKRWI |
| | | | YDTSKVASGVPYRFSGSGSGTSYSLTISSMEAEDAATYYCQQWSSNP |
| | | | LTFGSGTKLEINR |

TABLE 2
Amino acid sequences of mAb CDRs

| | | | SEQ |
|---|---|---|---|
| Accession No. | CDR | Sequence | ID NO: |
| Anti-CD2 VH | CDR1 | SYDMS | 88 |
| | CDR2 | YISGGGFTYYPDTVKG | 89 |
| | CDR3 | QGANWELVY | 90 |
| Anti-CD2 VL | CDR1 | RASQSISDFLH | 91 |
| | CDR2 | YASQSIS | 92 |
| | CDR3 | QNGHNFPPT | 93 |
| Anti-CD137 VH | CDR1 | GYYWS | 94 |
| | CDR2 | EINHGGYVTYNPSLES | 95 |
| | CDR3 | DYGPGNYDWYFDL | 96 |
| Anti-CD137 VL | CDR1 | RASQSVSSYLA | 97 |
| | CDR2 | DASNRAT | 98 |
| | CDR3 | QQRSNWPPALT | 99 |
| Anti-EGFR VH | CDR1 | NYGVH | 100 |
| | CDR2 | VIWSGGNTDYNTPFTS | 101 |
| | CDR3 | ALTYYDYEFAY | 102 |
| Anti-EGFR VL | CDR1 | RASQSIGTNIH | 103 |
| | CDR2 | YASESIS | 104 |
| | CDR3 | QQNNNWPTT | 105 |
| Anti-CD3 VH | CDR1 | RYTMH | 106 |
| | CDR2 | YINPSRGYTNYNQKFKD | 107 |
| | CDR3 | YYDDHYCLDY | 108 |
| Anti-CD3 VL | CDR1 | RASSSVSYMN | 109 |
| | CDR2 | DTSKVAS | 110 |
| | CDR3 | QQWSSNPLT | 111 |

### Example 6

### Tri-Specific Poly-Ig Proteins Maintain Affinity for their Targets

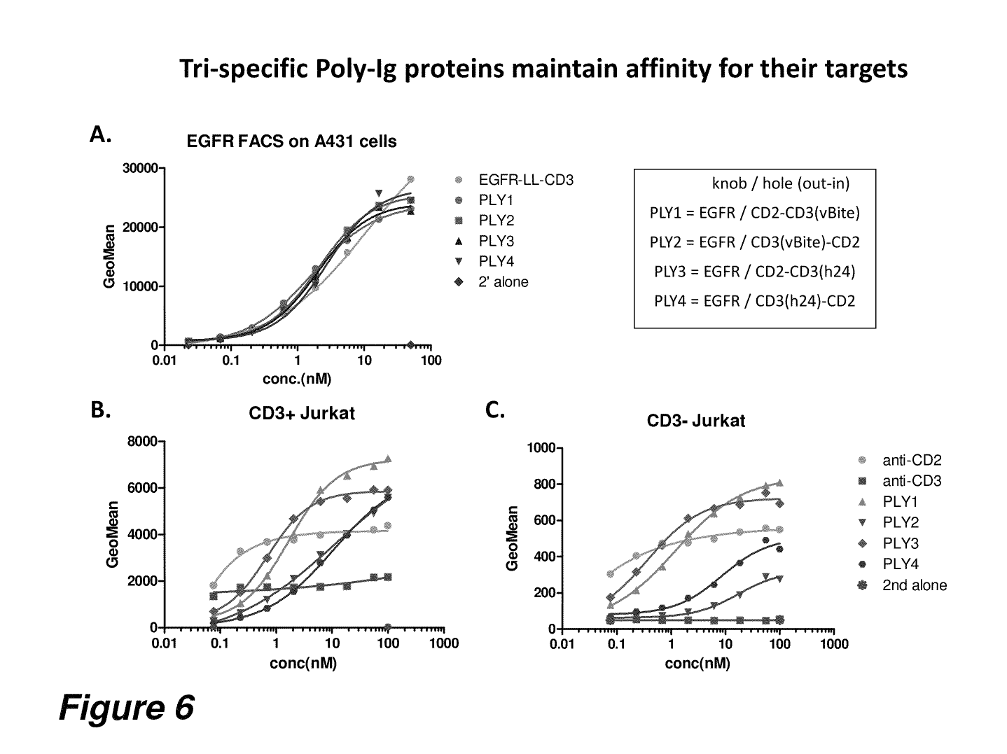

Tri-specific EGFR/CD2-CD3 and EGFR/CD3-CD2 Poly-Ig binding proteins were analyzed by FACS in Examples herein to determine whether the EGFR and CD3 binding domains maintained affinity for their cognate antigens (see FIG. 6). A431 cells, CD3⁺ Jurkat cels, or CD3⁻ Jurkat cells (0.5×10⁶) were washed and resuspended in FACS buffer (1% BSA/PBS) for use in the FACS assay. Monoclonal antibodies (mAbs) or Tri-specific proteins were prepared in 50 μl of FACS buffer, and mixed with cells, which were then incubated at 4° C. for 1 hour (hr). Cells were washed with FACS buffer and resuspended in 50 μl of FACS buffer containing Alexa Fluo 488-F(ab′)2 fragment goat anti-human IgG, Fcγ (Jackson ImmunoResearch, West Grove, Pa.). The resuspended were incubated at 4° C. for 1 hr. The labeled cells were washed with FACS buffer and analyzed in a FACSCanto II flow cytometer (BD Biosciences, San Jose, Calif.). It was observed that the Tri-specific proteins maintained affinity for their respective cognate antigens, indicating the binding functionality of these tri-specific proteins shown herein (FIGS. 6A-6C).

### Example 7

### Diminished Function of the Inner CD2 or CD3 Binding Domain

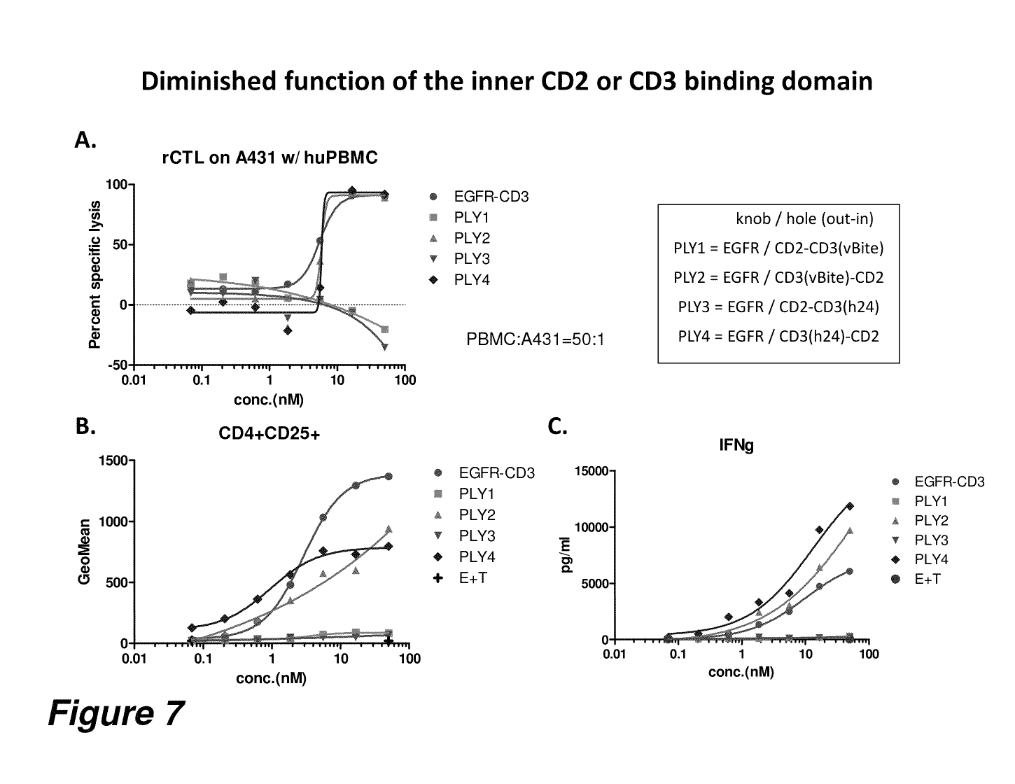

Tri-specific EGFR/CD2-CD3 and EGFR/CD3-CD2 Poly-Ig binding proteins were evaluated as described in Example 3, herein, to determine whether the functionality of the CD2 and CD3 domains was maintained in these constructs (see FIG. 7). It was observed that administering Poly-Ig proteins comprising inner CD3 binding domains (PLY1 and PLY3) reduced tumor cell killing, T-cell activation, and cytokine secretion compared administering an EGFR/CD3 DVD-Ig and Poly-Ig binding proteins comprising outer CD3 binding domains (PLY 2 and PLY4), as set forth in FIG. 7, herein. These data show that, though the inner CD3 binding domain was capable of binding its target, it does not function as effectively as a co-receptor in this position.

### Example 8

### Poly-Igs Comprising CD2 or CD137 (4-1BB) Domains on the Knob Chain

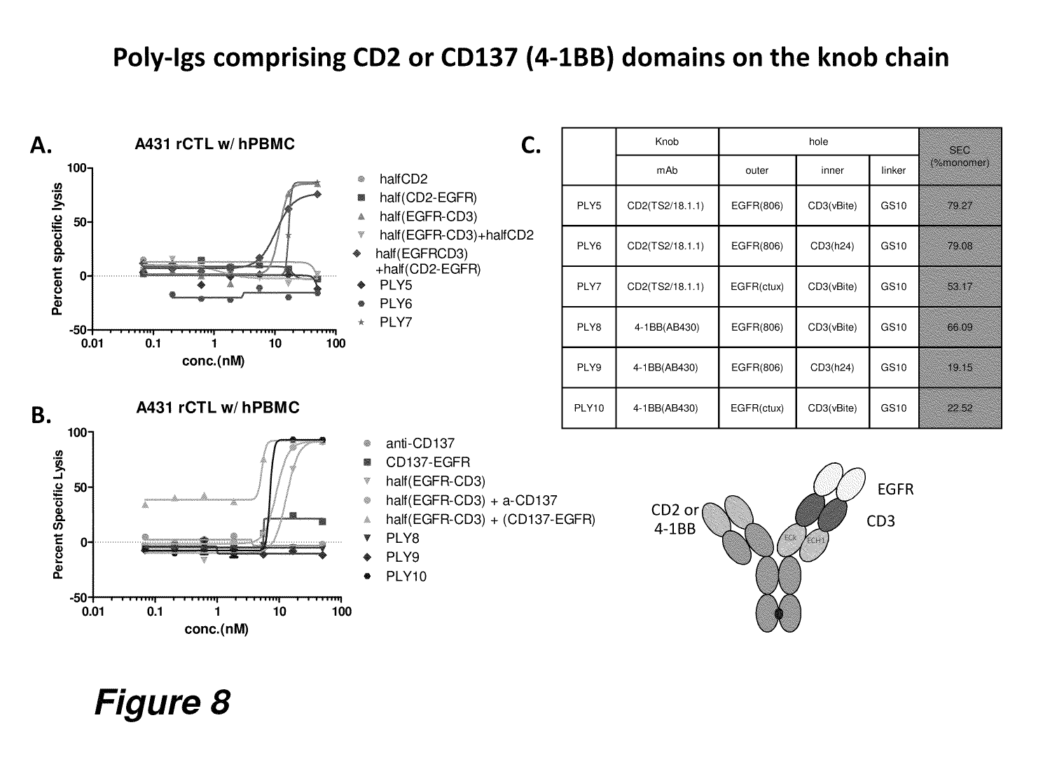

To improve the affinity and functionality of the Tri-specific Poly-Ig binding proteins, CD2 or CD137 (4-1BB) binding domains were placed on the knob chain and EGFR/CD3 DVD-Ig binding domains were placed on the hole chain (see FIG. 8). As set forth in FIGS. 8A-8C, though some of the Poly-Ig proteins maintained comparable tumor cell killing to EGFR/CD3 DVD-Ig proteins, all of the Poly-Ig proteins had demonstrably low SEC profiles.

### Example 9

### Poly-Igs have Improved SEC Profile and Function without the CH1/Ck Swap

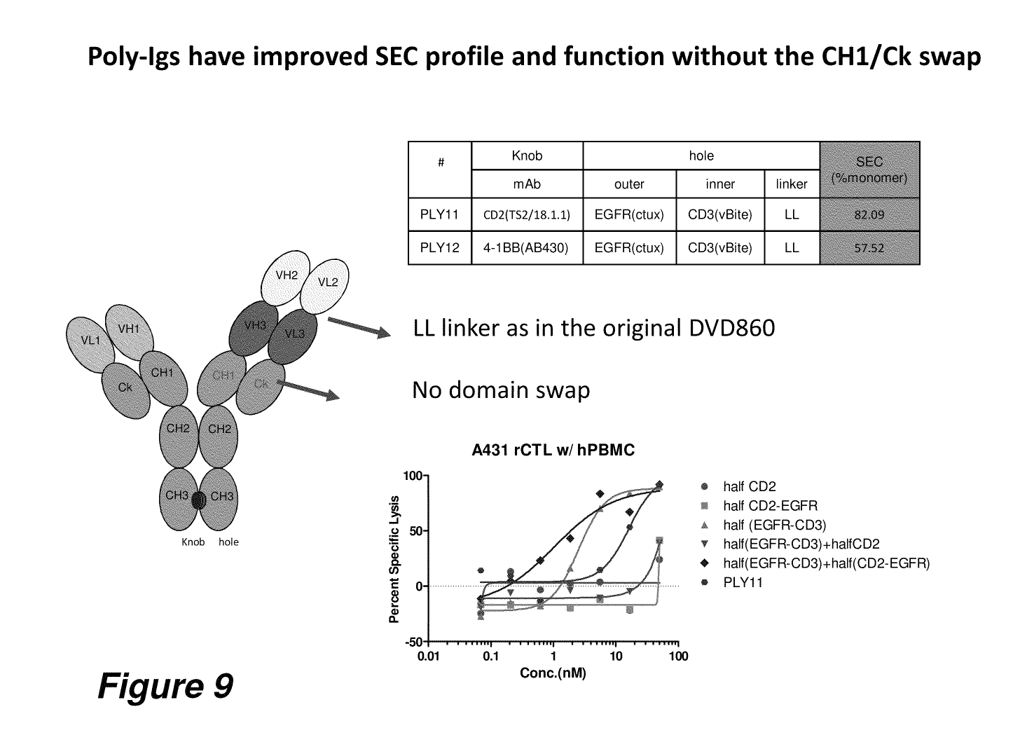

To improve the observed SEC profiles, Examples herein reversed the CH1/Ck domain swap to the wild type Fc domain orientation (see FIG. 9). As set forth in FIG. 9, the purified Tri-specific Poly-Ig proteins in the wild-type orientation showed improved SEC profiles compared to the Tri-specific Poly-Ig proteins in non-wild-type orientation. Furthermore the CD2/EGFR-CD3 (PLY11) protein had comparable tumor cell killing to EGFR/CD3 DVD-Ig proteins (see FIG. 9).

### Example 10

### PLY11 Reduced Cytokine Production while Maintaining Killing Activity

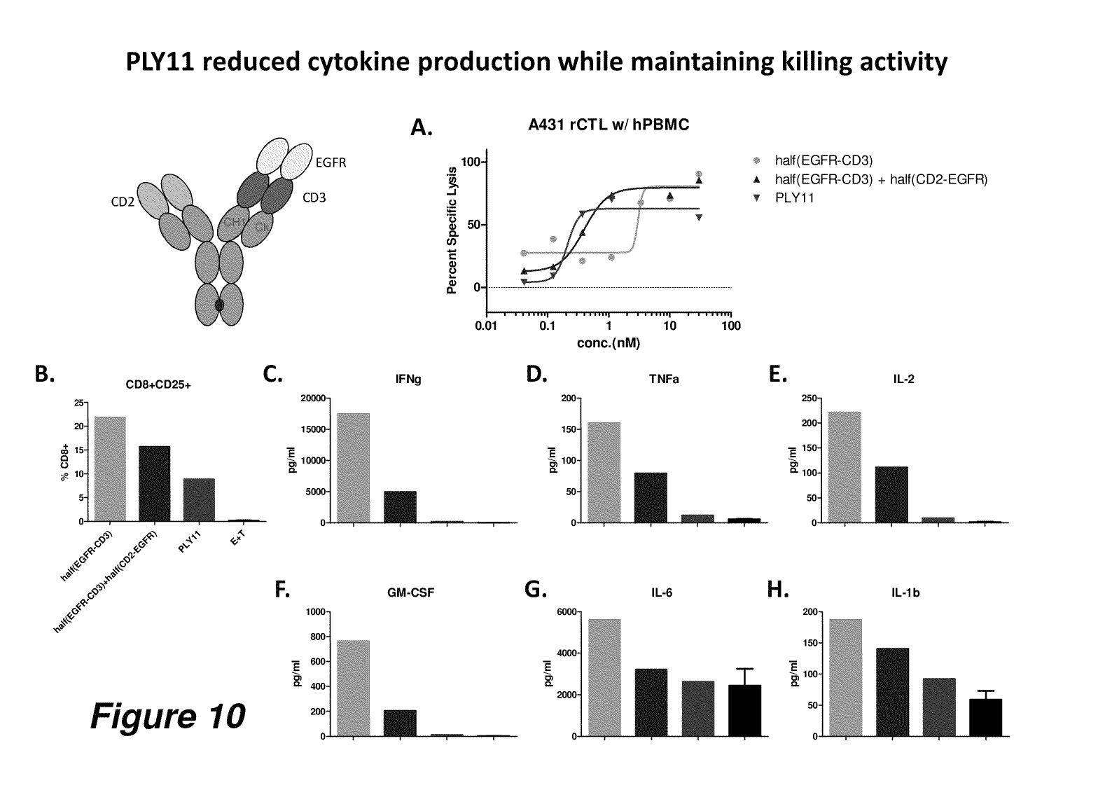

The Tri-specific CD2/EGFR-CD3 (PLY11) Poly-Ig binding protein was further evaluated as described in Example 3, herein, to determine whether the functionality of the CD2, EGFR, and CD3 domains was maintained in these constructs (see FIG. 10). PLY11 binding protein was analyzed using an rCTL killing assay, and T-cell activation and cytokine release were measured. As set forth in FIGS. 10A-10H, the PLY11 binding protein demonstrated comparable tumor cell killing to EGFR/CD3 DVD-Ig proteins, and most importantly reduced T-cell activation and cytokine secretion compared with controls.

### Example 11

### PLY12 Enhanced T-Cell Activation

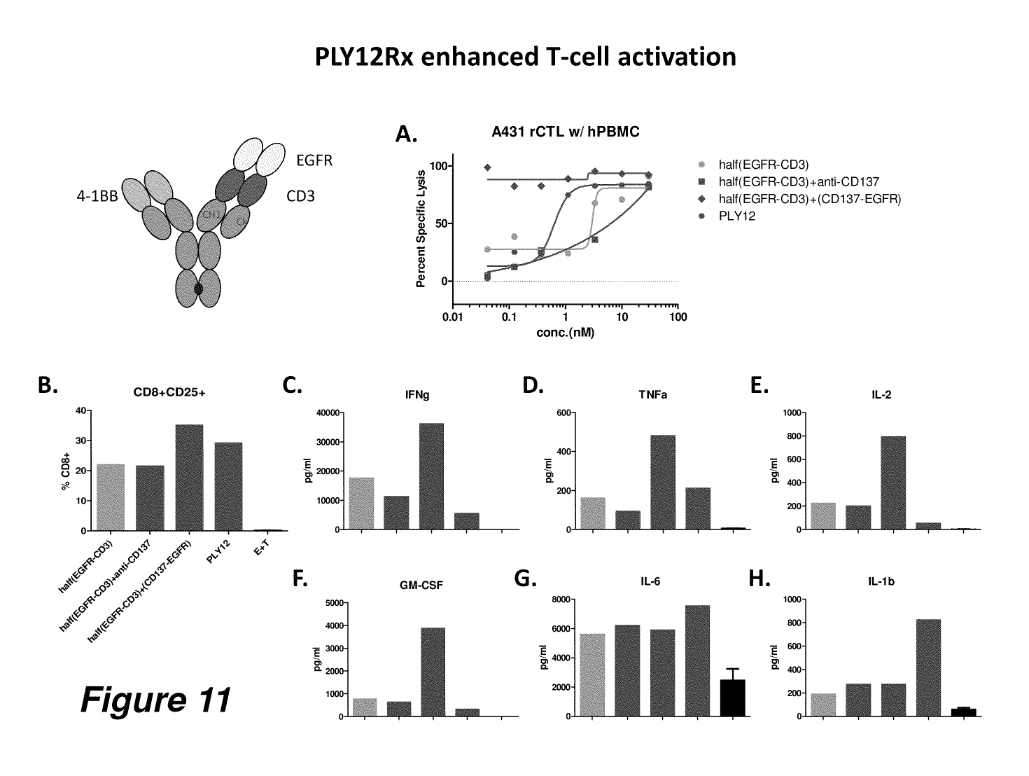

The Tri-specific CD123 (4-1BB)/EGFR-CD3 (PLY12) Poly-Ig binding protein was evaluated as described in Example 3, herein, to determine whether the functionality of the CD137, EGFR, and CD3 domains was maintained in these constructs (see FIG. 11). PLY12 was analyzed using rCTL killing assay and T-cell activation and cytokine release were measured. PLY12 treated samples demonstrated improved tumor cell killing, comparable T-cell activation, and similar levels of some secreted cytokines compared to cells treated with EGFR/CD3 DVD-Ig proteins (FIGS. 11A-11H). Thus, PLY12 is a potential therapeutic composition for use in treating diseases in which T-cell modulation and/or activation is beneficial.

### Example 12

### DVD-Ig Binding to Two Co-Stimulatory Molecules on Activated Human T-Cells

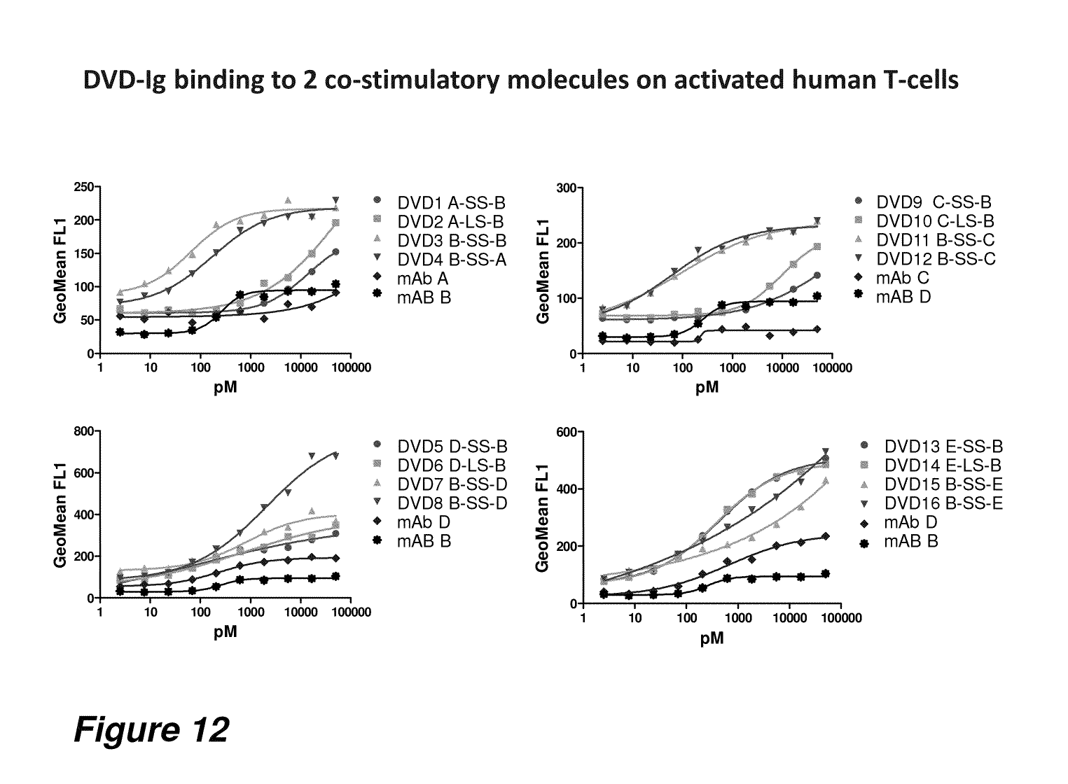

DVD-Ig binding proteins capable of binding two T-cell co-stimulatory molecules were constructed from parental antibodies using art known methods. The amino acid sequences of the parental antibodies and their CDRs are shown in Tables 3 and 4. The amino acid sequences of the DVD-Ig heavy and light chains are set forth in Table 5. The functional affinity of various DVD-Ig molecules and antibodies to bind to co-stimulatory molecules on activated human T-cells was assessed using a FACS-based binding assay (see FIG. 12). Plots of Geomean against antibody/DVD concentration are shown in FIG. 12, herein. These data demonstrate that DVD-Ig molecules that simultaneously bound to two co-stimulatory molecules on activated T-cells exhibited a greater functional affinity for the T-cells than monospecific antibodies that bind to the same co-stimulatory molecules individually.

TABLE 3
Amino acid sequences of parental mAb VH/VL regions

| | Antibody | Variable | |
|---|---|---|---|
| SEQ ID | Name | Domain | Sequence |
| 112. | PD1 | VH | QVQLVESGGGVVQPGRSLRLDCKASGITFSNSGMHWVRQAPGKG |
| | | | LEWVAVIWYDGSKRYYADSVKGRFTISRDNSKNTLFLQMNSLRA |
| | | | EDTAVYYCATNDDYWGQGTLVTVSS |
| 113. | PD1 | VL | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQSSNWPRTFGQGTKVEIKR |
| 114. | CTLA4 | VH | QVQLVESGGGVVQPGRSLRLSCAASGFTFSSYTMHWVRQAPGKG |
| | | | LEWVTFISYDGNNKYYADSVKGRFTISRDNSKNTLYLQMNSLRA |
| | | | EDTAIYYCARTGWLGPFDYWGQGTLVTVSS |
| 115. | CTLA4 | VL | EIVLTQSPGTLSLSPGERATLSCRASQSVGSSYLAWYQQKPGQA |
| | | | PRLLIYGAFSRATGIPDRFSGSGSGTDFTLTISRLEPEDFAVYY |
| | | | CQQYGSSPWTFGQGTKVEIKR |
| 116. | LAG3 | VH | QVQLQQWGAGLLKPSETLSLTCAVYGGSFSDYYWNWIRQPPGKG |
| | | | LEWIGEINHNGNTNSNPSLKSRVTLSLDTSKNQFSLKLRSVTAA |
| | | | DTAVYYCAFGYSDYEYNWFDPWGQGTLVTVSS |
| 117. | LAG3 | VL | EIVLTQSPATLSLSPGERATLSCRASQSISSYLAWYQQKPGQAP |
| | | | RLLIYDASNRATGIPARFSGSGSGTDFTLTISSLEPEDFAVYYC |
| | | | QQRSNWPLTFGQGTNLEIKR |
| 118. | 41BB | VH | QVQLQQWGAGLLKPSETLSLTCAVYGGSFSGYYWSWIRQSPEKG |
| | | | LEWIGEINHGGYVTYNPSLESRVTISVDTSKNQFSLKLSSVTAA |
| | | | DTAVYYCARDYGPGNYDWYFDLWGRGTLVTVSS |
| 119. | 41BB | VL | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQRSNWPPALTFGGGTKVEIKR |

TABLE 4
Amino acid sequences of parental mAb CDRs

| | | | SEQ |
|---|---|---|---|
| Antibody | CDR | Sequence | ID NO: |
| PD1VH | CDR1 | GITFSNSGMH | 120. |
| | CDR2 | VIWYDGSKRYYADSVKG | 121. |
| | CDR3 | NDDY | 122. |
| PD1VL | CDR1 | RASQSVSSYLA | 123. |
| | CDR2 | YDASNRA | 124. |
| | CDR3 | QQSSNWPRT | 125. |
| CTLA4VH | CDR1 | GFTFSSYTMH | 126. |
| | CDR2 | FISYDGNNKYYADSVKG | 127. |
| | CDR3 | TGWLGPFDY | 128. |
| CTLA4VL | CDR1 | QSVGSSYLA | 129. |
| | CDR2 | YGAFSRA | 130. |
| | CDR3 | QQYGSSPWT | 131. |
| LAG3VH | CDR1 | GGSFSDYYWN | 132. |
| | CDR2 | EINHNGNTNSNPSLKS | 133. |
| | CDR3 | GYSDYEYNWFDP | 134. |
| LAG3VL | CDR1 | QSISSYLA | 135. |
| | CDR2 | YDASNRA | 136. |
| | CDR3 | QQRSNWPLT | 137. |
| 41BBVH | CDR1 | GGSFSGYYWS | 138. |
| | CDR2 | EINHGGYVTYNPSLES | 139. |
| | CDR3 | DYGPGNYDWYFDL | 140. |
| 41BBVL | CDR1 | QSVSSYLA | 141. |
| | CDR2 | YDASNRA | 142. |
| | CDR3 | QQRSNWPPALT | 143. |

TABLE 5
Amino acid sequences of DVD-Ig heavy and light chains

| | DVD | Outer | | Inner | |
|---|---|---|---|---|---|
| | Variable | Variable | | Variable | |
| SEQ | Domain | Domain | | Domain | |
| ID NO | Name | Name | Linker | Name | Sequence |
| 144. | DVD2555H | PD1VH | HG-short | LAG3VH | QVQLVESGGGVVQPGRSLRLDCKASGITFS |
| | | | | | NSGMHWVRQAPGKGLEWVAVIWYDGSKRYY |
| | | | | | ADSVKGRFTISRDNSKNTLFLQMNSLRAED |
| | | | | | TAVYYCATNDDYWGQGTLVTVSSastkgpQ |
| | | | | | VQLQQWGAGLLKPSETLSLTCAVYGGSFSD |
| | | | | | YYWNWIRQPPGKGLEWIGEINHNGNTNSNP |
| | | | | | SLKSRVTLSLDTSKNQFSLKLRSVTAADTA |
| | | | | | VYYCAFGYSDYEYNWFDPWGQGTLVTVSS |
| 145. | DVD2555L | PD1VL | LK-short | LAG3VL | EIVLTQSPATLSLSPGERATLSCRASQSVS |
| | | | | | SYLAWYQQKPGQAPRLLIYDASNRATGIPA |
| | | | | | RFSGSGSGTDFTLTISSLEPEDFAVYYCQQ |
| | | | | | SSNWPRTFGQGTKVEIKRtvaapEIVLTQS |
| | | | | | PATLSLSPGERATLSCRASQSISSYLAWYQ |
| | | | | | QKPGQAPRLLIYDASNRATGIPARFSGSGS |
| | | | | | GTDFTLTISSLEPEDFAVYYCQQRSNWPLT |
| | | | | | FGQGTNLEIKR |
| 146. | DVD2556H | PD1VH | HG-long | LAG3VH | QVQLVESGGGVVQPGRSLRLDCKASGITFS |
| | | | | | NSGMHWVRQAPGKGLEWVAVIWYDGSKRYY |
| | | | | | ADSVKGRFTISRDNSKNTLFLQMNSLRAED |
| | | | | | TAVYYCATNDDYWGQGTLVTVSSastkgps |
| | | | | | vfplapQVQLQQWGAGLLKPSETLSLTCAV |
| | | | | | YGGSFSDYYWNWIRQPPGKGLEWIGEINHN |
| | | | | | GNTNSNPSLKSRVTLSLDTSKNQFSLKLRS |
| | | | | | VTAADTAVYYCAFGYSDYEYNWFDPWGQGT |
| | | | | | LVTVSS |
| 147. | DVD2556L | PD1VL | LK-short | LAG3VL | EIVLTQSPATLSLSPGERATLSCRASQSVS |
| | | | | | SYLAWYQQKPGQAPRLLIYDASNRATGIPA |
| | | | | | RFSGSGSGTDFTLTISSLEPEDFAVYYCQQ |
| | | | | | SSNWPRTFGQGTKVEIKRtvaapEIVLTQS |
| | | | | | PATLSLSPGERATLSCRASQSISSYLAWYQ |
| | | | | | QKPGQAPRLLIYDASNRATGIPARFSGSGS |
| | | | | | GTDFTLTISSLEPEDFAVYYCQQRSNWPLT |
| | | | | | FGQGTNLEIKR |
| 148. | DVD2557H | LAG3VH | HG-short | PD1VH | QVQLQQWGAGLLKPSETLSLTCAVYGGSFS |
| | | | | | DYYWNWIRQPPGKGLEWIGEINHNGNTNSN |
| | | | | | PSLKSRVTLSLDTSKNQFSLKLRSVTAADT |
| | | | | | AVYYCAFGYSDYEYNWFDPWGQGTLVTVSS |
| | | | | | astkgpQVQLVESGGGVVQPGRSLRLDCKA |
| | | | | | SGITFSNSGMHWVRQAPGKGLEWVAVIWYD |
| | | | | | GSKRYYADSVKGRFTISRDNSKNTLFLQMN |
| | | | | | SLRAEDTAVYYCATNDDYWGQGTLVTVSS |
| 149. | DVD2557L | LAG3VL | LK-short | PD1VL | EIVLTQSPATLSLSPGERATLSCRASQSIS |
| | | | | | SYLAWYQQKPGQAPRLLIYDASNRATGIPA |
| | | | | | RFSGSGSGTDFTLTISSLEPEDFAVYYCQQ |
| | | | | | RSNWPLTFGQGTNLEIKRtvaapEIVLTQS |
| | | | | | PATLSLSPGERATLSCRASQSVSSYLAWYQ |
| | | | | | QKPGQAPRLLIYDASNRATGIPARFSGSGS |
| | | | | | GTDFTLTISSLEPEDFAVYYCQQSSNWPRT |
| | | | | | FGQGTKVEIKR |
| 150. | DVD2558H | LAG3VH | HG-long | PD1VH | QVQLQQWGAGLLKPSETLSLTCAVYGGSFS |
| | | | | | DYYWNWIRQPPGKGLEWIGEINHNGNTNSN |
| | | | | | PSLKSRVTLSLDTSKNQFSLKLRSVTAADT |
| | | | | | AVYYCAFGYSDYEYNWFDPWGQGTLVTVSS |
| | | | | | astkgpsvfplapQVQLVESGGGVVQPGRS |
| | | | | | LRLDCKASGITFSNSGMHWVRQAPGKGLEW |
| | | | | | VAVIWYDGSKRYYADSVKGRFTISRDNSKN |
| | | | | | TLFLQMNSLRAEDTAVYYCATNDDYWGQGT |
| | | | | | LVTVSS |
| 151. | DVD2558L | LAG3VL | LK-short | PD1VL | EIVLTQSPATLSLSPGERATLSCRASQSIS |
| | | | | | SYLAWYQQKPGQAPRLLIYDASNRATGIPA |
| | | | | | RFSGSGSGTDFTLTISSLEPEDFAVYYCQQ |
| | | | | | RSNWPLTFGQGTNLEIKRtvaapEIVLTQS |
| | | | | | PATLSLSPGERATLSCRASQSVSSYLAWYQ |
| | | | | | QKPGQAPRLLIYDASNRATGIPARFSGSGS |
| | | | | | GTDFTLTISSLEPEDFAVYYCQQSSNWPRT |
| | | | | | FGQGTKVEIKR |
| 152. | DVD2567H | 41BBVH | HG-short | LAG3VH | QVQLQQWGAGLLKPSETLSLTCAVYGGSFS |
| | | | | | GYYWSWIRQSPEKGLEWIGEINHGGYVTYN |
| | | | | | PSLESRVTISVDTSKNQFSLKLSSVTAADT |
| | | | | | AVYYCARDYGPGNYDWYFDLWGRGTLVTVS |
| | | | | | SastkgpQVQLQQWGAGLLKPSETLSLTCA |
| | | | | | VYGGSFSDYYWNWIRQPPGKGLEWIGEINH |
| | | | | | NGNTNSNPSLKSRVTLSLDTSKNQFSLKLR |
| | | | | | SVTAADTAVYYCAFGYSDYEYNWFDPWGQG |
| | | | | | TLVTVSS |
| 153. | DVD2567L | 41BBVL | LK-short | LAG3VL | EIVLTQSPATLSLSPGERATLSCRASQSVS |
| | | | | | SYLAWYQQKPGQAPRLLIYDASNRATGIPA |
| | | | | | RFSGSGSGTDFTLTISSLEPEDFAVYYCQQ |
| | | | | | RSNWPPALTFGGGTKVEIKRtvaapEIVLT |
| | | | | | QSPATLSLSPGERATLSCRASQSISSYLAW |
| | | | | | YQQKPGQAPRLLIYDASNRATGIPARFSGS |
| | | | | | GSGTDFTLTISSLEPEDFAVYYCQQRSNWP |
| | | | | | LTFGQGTNLEIKR |
| 154. | DVD2568H | 41BBVH | HG-long | LAG3VH | QVQLQQWGAGLLKPSETLSLTCAVYGGSFS |
| | | | | | GYYWSWIRQSPEKGLEWIGEINHGGYVTYN |
| | | | | | PSLESRVTISVDTSKNQFSLKLSSVTAADT |
| | | | | | AVYYCARDYGPGNYDWYFDLWGRGTLVTVS |
| | | | | | SastkgpsvfplapQVQLQQWGAGLLKPSE |
| | | | | | TLSLTCAVYGGSFSDYYWNWIRQPPGKGLE |
| | | | | | WIGEINHNGNTNSNPSLKSRVTLSLDTSKN |
| | | | | | QFSLKLRSVTAADTAVYYCAFGYSDYEYNW |
| | | | | | FDPWGQGTLVTVSS |
| 155. | DVD2568L | 41BBVL | LK-short | LAG3VL | EIVLTQSPATLSLSPGERATLSCRASQSVS |
| | | | | | SYLAWYQQKPGQAPRLLIYDASNRATGIPA |
| | | | | | RFSGSGSGTDFTLTISSLEPEDFAVYYCQQ |
| | | | | | RSNWPPALTFGGGTKVEIKRtvaapEIVLT |
| | | | | | QSPATLSLSPGERATLSCRASQSISSYLAW |
| | | | | | YQQKPGQAPRLLIYDASNRATGIPARFSGS |
| | | | | | GSGTDFTLTISSLEPEDFAVYYCQQRSNWP |
| | | | | | LTFGQGTNLEIKR |
| 156. | DVD2569H | LAG3VH | HG-short | 41BBVH | QVQLQQWGAGLLKPSETLSLTCAVYGGSFS |
| | | | | | DYYWNWIRQPPGKGLEWIGEINHNGNTNSN |
| | | | | | PSLKSRVTLSLDTSKNQFSLKLRSVTAADT |
| | | | | | AVYYCAFGYSDYEYNWFDPWGQGTLVTVSS |
| | | | | | astkgpQVQLQQWGAGLLKPSETLSLTCAV |
| | | | | | YGGSFSGYYWSWIRQSPEKGLEWIGEINHG |
| | | | | | GYVTYNPSLESRVTISVDTSKNQFSLKLSS |
| | | | | | VTAADTAVYYCARDYGPGNYDWYFDLWGRG |
| | | | | | TLVTVSS |
| 157. | DVD2569L | LAG3VL | LK-short | 41BBVL | EIVLTQSPATLSLSPGERATLSCRASQSIS |
| | | | | | SYLAWYQQKPGQAPRLLIYDASNRATGIPA |
| | | | | | RFSGSGSGTDFTLTISSLEPEDFAVYYCQQ |
| | | | | | RSNWPLTFGQGTNLEIKRtvaapEIVLTQS |
| | | | | | PATLSLSPGERATLSCRASQSVSSYLAWYQ |
| | | | | | QKPGQAPRLLIYDASNRATGIPARFSGSGS |
| | | | | | GTDFTLTISSLEPEDFAVYYCQQRSNWPPA |
| | | | | | LTFGGGTKVEIKR |
| 158. | DVD2570H | LAG3VH | HG-long | 41BBVH | QVQLQQWGAGLLKPSETLSLTCAVYGGSFS |
| | | | | | DYYWNWIRQPPGKGLEWIGEINHNGNTNSN |
| | | | | | PSLKSRVTLSLDTSKNQFSLKLRSVTAADT |
| | | | | | AVYYCAFGYSDYEYNWFDPWGQGTLVTVSS |
| | | | | | astkgpsvfplapQVQLQQWGAGLLKPSET |
| | | | | | LSLTCAVYGGSFSGYYWSWIRQSPEKGLEW |
| | | | | | IGEINHGGYVTYNPSLESRVTISVDTSKNQ |
| | | | | | FSLKLSSVTAADTAVYYCARDYGPGNYDWY |
| | | | | | FDLWGRGTLVTVSS |
| 159. | DVD2570L | LAG3VL | LK-short | 41BBVL | EIVLTQSPATLSLSPGERATLSCRASQSIS |
| | | | | | SYLAWYQQKPGQAPRLLIYDASNRATGIPA |
| | | | | | RFSGSGSGTDFTLTISSLEPEDFAVYYCQQ |
| | | | | | RSNWPLTFGQGTNLEIKRtvaapEIVLTQS |
| | | | | | PATLSLSPGERATLSCRASQSVSSYLAWYQ |
| | | | | | QKPGQAPRLLIYDASNRATGIPARFSGSGS |
| | | | | | GTDFTLTISSLEPEDFAVYYCQQRSNWPPA |
| | | | | | LTFGGGTKVEIKR |

### Example 13

### Suppression of Cytokine Release by Human T-Cells by Engagement of Two Co-Repressors Simultaneously

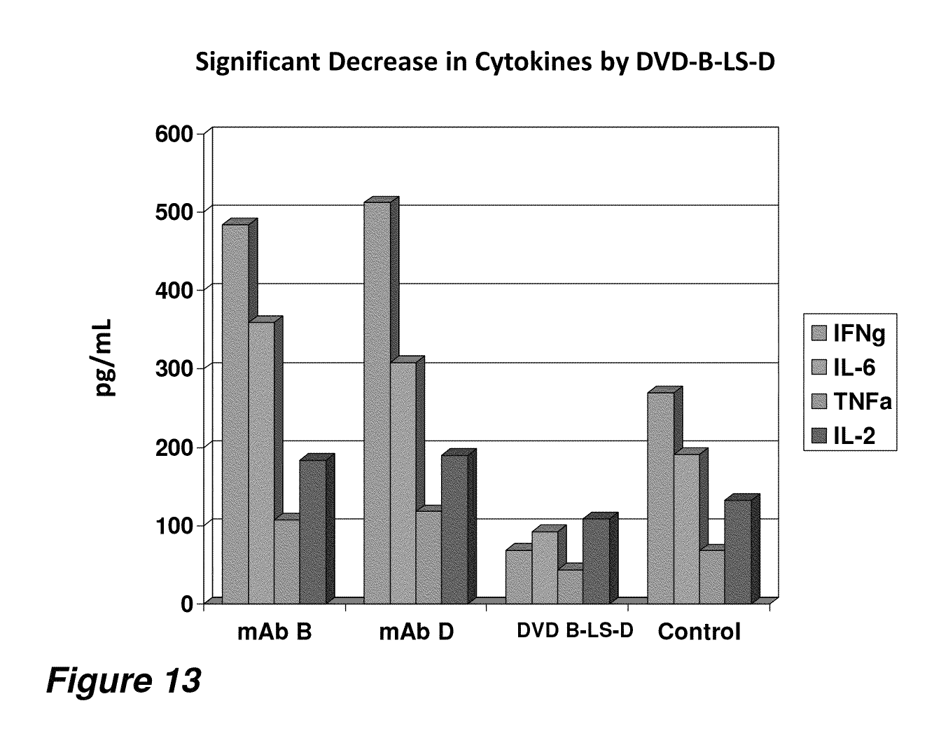

The effect of co-repressor engagement by monospecific antibodies or a bispecific DVD-Ig was assessed in Examples herein. Specifically, human T-cells were co-cultured with irradiated CD14+ derived dendritic cells in the presence of the antibodies, DVD-Ig, or control IgG. The amount of IFNγ, IL-6, TNFα, and IL-2 produced/expressed by the T-cells was measured (see FIG. 13). A monoclonal antibody that binds to co-repressor B (MAb B) and monoclonal antibody that binds to co-repressor D (MAb D) were used. Also prepared and used was a DVD-Ig binds to both co-repressors B and D (DVD-B-LS-D) simultaneously. The cytokine release data set forth in FIG. 13 indicates that, as expected, MAb B or MAb D enhanced T-cell cytokine release compared administering the IgG control. However, it was observed that administering DVD-B-LS-D resulted in greater suppression of T-cell cytokine release compared to administering IgG control. Accordingly, these data identify the simultaneous engagement of co-repressors B and D as an effective treatment for suppressing T-cell cytokine release.

### Example 14

### Generation and Characterization of DVD-Ig Binding Proteins Capable of Binding Two T-Cell Co-Stimulatory Molecules

Four-chain DVD-Ig binding proteins comprising parent antibodies with known amino acid sequences and demonstrated affinity for T-cell co-stimulatory molecules were generated by synthesizing polynucleotide fragments encoding DVD-Ig binding protein variable heavy chain and DVD-Ig binding protein variable light chain sequences and cloning the fragments into a pHybE-D2 vector according to art known methods. The DVD-Ig binding protein constructs were cloned into and expressed in 293 cells and purified according to art known methods. DVD-Ig VH and VL chains for the DVD binding proteins, as well as selected CDR sequences are provided in Table 4.

14.1: Preparation of Activated T-Cells and Flow Cytometry Analysis.

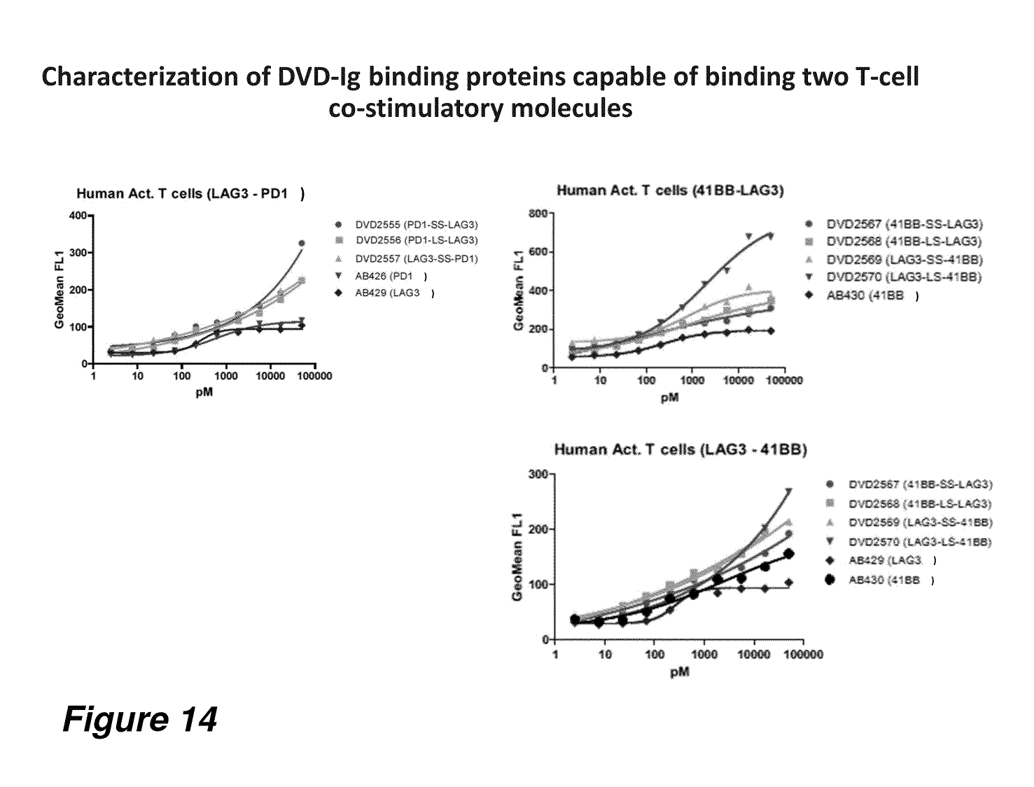

PBMCs were isolated from heparinized leukopaks by use of Ficoll density gradient centrifugation. PBMCs were stimulated overnight with CD2/CD3/CD28 beads from a T-Cell Activation/Expansion Kit (Miltenyi Biotech, Germany) according to the manufacturer's protocol. Activated T-cells were incubated with primary antibody for 1 hour at 4° C., washed in PBS, and then incubated for 30 minutes with fluorescently-labeled secondary anti-Fc specific antibody (Jackson ImmunoResearch, West Grove, Pa.). Activated T-cell samples were analyzed on a FACSCantoII cytometer (BD) and data were analyzed with FlowJo software. Geometric mean fluorescence intensity was compared to DVD-Ig protein concentration by graphing in the GraphPad Prism software package (see FIG. 14).

Plots of Geomean against antibody/DVD concentration are set forth in FIGS. 14A-14C, herein. DVD-Ig molecules that simultaneously bind to two co-stimulatory molecules on activated T-cells exhibited a greater functional affinity for the T-cells than monospecific antibodies that bind to the same co-stimulatory molecules individually. Both variable domains in DVD2555, DVD2556, DVD2557, DVD2567, DVD2568, DVD2569, and DVD25707 were capable of binding the intended T-cell surface marker. It was observed that DVD2555 and DVD2570 bound activated T-cells with an greater affinity than similar DVD-Ig proteins. Together these data indicate that binding affinity is adaptable in part, by the particular orientation of the variable domains and/or the linker peptides between the variable domains. These data identify DVD2555 and DVD2570 as potential therapeutic compositions for use in diseases in which T-cell modulation and/or activation is beneficial.

### Example 15

### Mixed Lymphocyte Reaction (MLR) to Measure T-Cell Activity

15.1 Monocyte Isolation and Dendritic Cell Differentiation

Using CD14 MicroBeads (Miltenyi Biotech, Germany) monocytes were isolated according to manufactures instructions. Monocytes were cultured for 5 days in GM-CSF and human IL-4 (R&D) to induce differentiation into dendritic cells. Dendritic cells were matured by culturing in 1 μg/mL LPS (Sigma) overnight.

15.2 Measuring T-Cell Proliferation in MLR

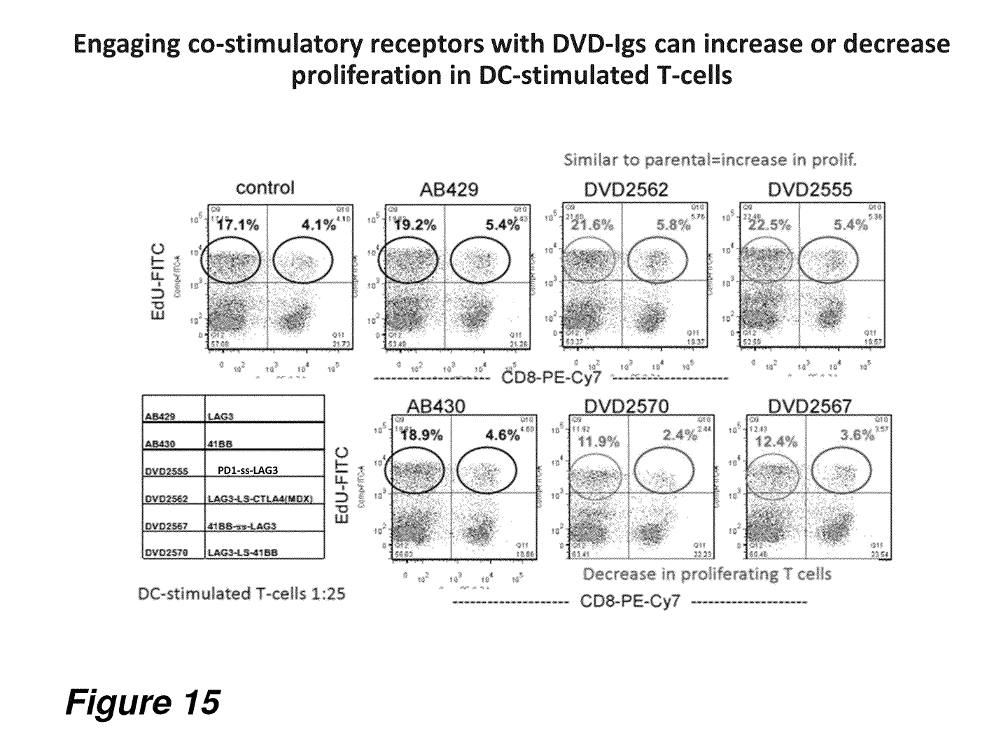

T-cells were cultured with irradiated dendritic cells at a 25:1 ratio for 5 days. Proliferation was measured by flow cytometry using the Click-iT EdU reagent (Life Tech), according to manufacturer's recommendations. FACS staining for T-cell markers CD4, CD8, CD25 and CD69 (eBioscience antibodies) were carried out simultaneously (see FIG. 15). Supernatants were kept for cytokine analysis by multiplex MSD (Meso-scale).

CD8+ T-cell proliferation was characterized as set forth in FIG. 15, herein. The upper right quadrant in FIG. 15 shows percentage of CD8+ T-cells incorporating the EdU dye as a measurement of proliferation. The anti-LAG3 co-inhibitory receptor and the anti-4-1BB co-stimulatory receptor antibodies showed evidence of increased proliferation as compared to the untreated control MLR. Interestingly, the DVD-Ig molecules DVD2567 and DVD2570 containing the combination of these two parental domains showed inhibited T-cell proliferation and activation (data not shown). Combination of two co-inhibitory receptor antibodies in a DVD-Ig so as to produce an additive effect is shown herein. For example data show that DVD2555 and DVD2562 resulted in enhanced T-cell proliferation compared to data using the parental antibodies.

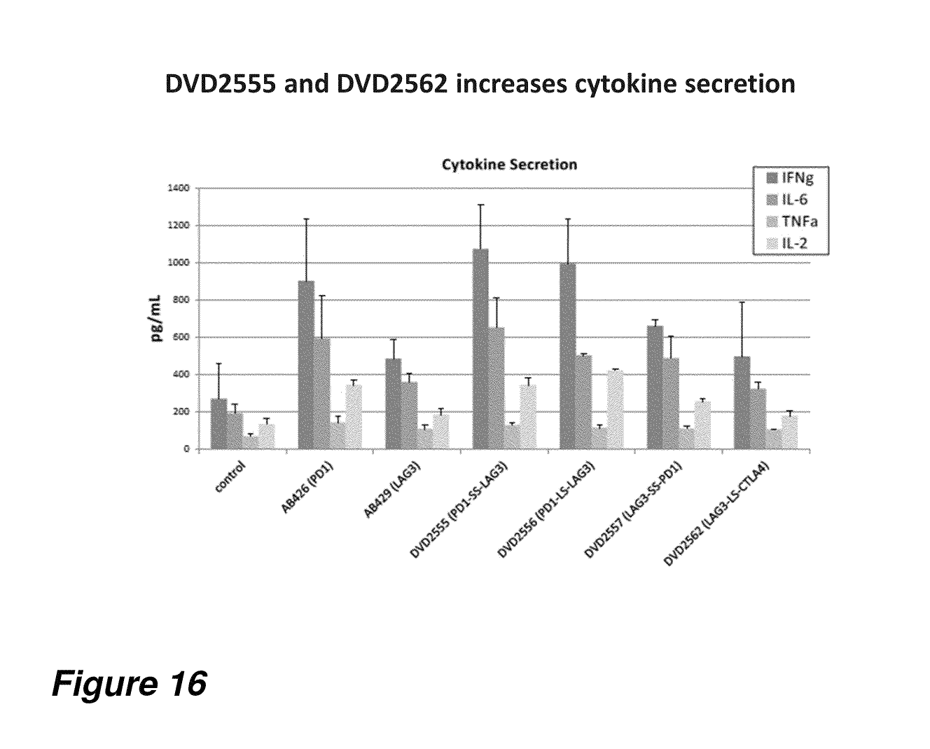

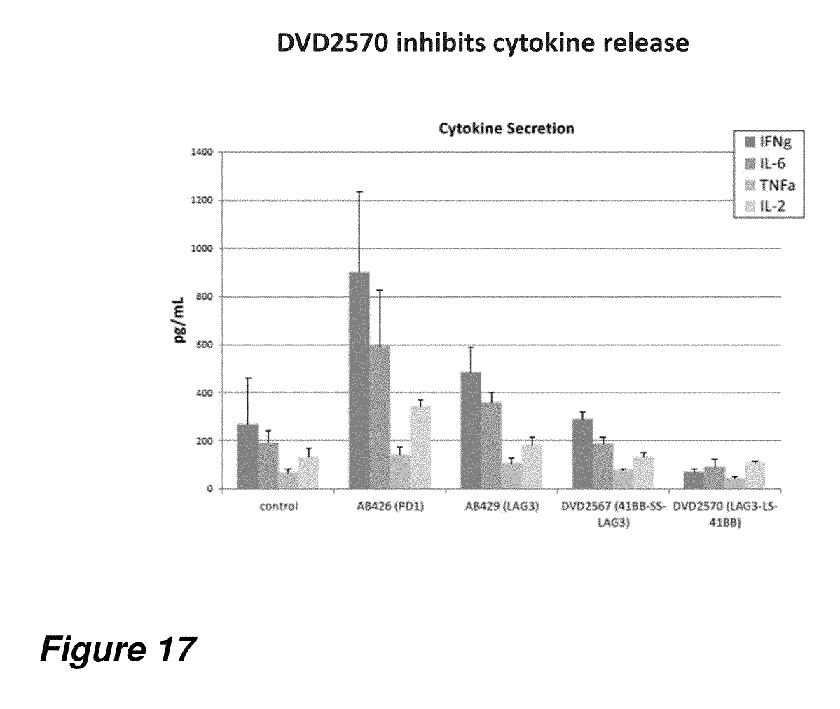

Assays measuring cytokines IFNγ, IL-6, TNFα, and IL-2 in MLR supernatants demonstrated the additive or synergistic effect of DVD-Ig molecules herein such as DVD2555 and DVD2562 (FIG. 16). The increase in T-cell activity was observed to be most pronounced in the IFNγ levels elicited by these DVD-Ig molecules DVD2555 and DVD2562. Furthermore a substantial reduction in the IFNγ levels was observed in the MLR supernatants contacted with DVD2570 (FIG. 17). Accordingly, these data show that the simultaneous engagement of T-cell co-receptors using binding proteins described herein is an effective treatment to modulate T-cell activation and cytokine release.

Other embodiments or variants as afforded by law, for example, under the doctrine of equivalents, are encompassed by the claims and supported by the specification as set forth herein.

1. A multispecific binding protein comprising a first binding site that specifically binds to a target cell antigen, a second binding site that specifically binds to a cell surface receptor on an immune cell, and a third binding site that specifically binds to cell surface modulator on the immune cell.

2. The multispecific binding protein of claim 1, wherein the immune cell is a myeloid cell, a lymphoid cell; an effector cell, a T-cell, a macrophage, a dendritic cell, a natural killer cell, an eosinophil, or a cytotoxic T-cell.

3-5. (canceled)

6. The multispecific binding protein of claim 1, wherein the cell surface receptor on the immune cell is a T-cell receptor (TCR) complex component.

7. The multispecific binding protein of claim 1, comprising a first binding site that specifically binds to a target cell antigen, a second binding site that specifically binds to a T-cell receptor (TCR) complex component on a T-cell, and a third binding site that specifically binds to a T-cell modulator on the T-cell.

8. The multispecific binding protein of claim 1, wherein the target cell antigen is a disease-associated antigen.

9. The multispecific binding protein of claim 1, wherein the target cell antigen is tumor-associated antigen.

10. (canceled)

11. The multispecific binding protein of claim 6, wherein the TCR complex component is CD3γ, CD3δ, or CD3ε.

12. (canceled)

13. The multispecific binding protein of claim 1, wherein the modulator is a stimulator of T-cell activation.

14. The multispecific binding protein of claim 1, wherein the modulator is an inhibitor of T-cell activation.

15-17. (canceled)

18. The multispecific binding protein of claim 1 which is a DVD-Ig, half-DVD-Ig, a scDVD-Ig, an fDVD-Ig, an rDVD-Ig, a pDVD-Ig, an mDVD-Ig or a coDVD-Ig.

19. (canceled)

20. The method of claim 18, wherein the pDVD-Ig comprises first, second, third and fourth polypeptide chains,
wherein said first polypeptide chain comprises VD1-(X1)n-VD2-C-(X2)n, wherein VD1 is a first heavy chain variable domain, VD2 is a second heavy chain variable domain, C is a constant domain, X1 is a linker with the proviso that it is not a constant domain, and X2 is an Fc region;
wherein said second polypeptide chain comprises VD3-(X1)n-VD4-C-(X2)n, wherein VD3 is a first light chain variable domain, VD4 is a second light chain variable domain, C is a constant domain, X1 is a linker with the proviso that it is not a constant domain, and X2 does not comprise an Fc region;
wherein said third polypeptide chain comprises VD5-C-(X3)n, wherein VD5 is a third heavy chain variable domain, C is a constant domain, and X3 is an Fc region;
wherein said fourth polypeptide chain comprises VD6-C-(X3)n, wherein VD6 is a third light chain variable domain, C is a constant domain, and X4 does not comprise an Fc region;
wherein n is 0 or 1, and wherein the VD1 and VD3 domains on the first and second polypeptide chains form one functional binding site for a first antigen, the VD2 and VD4 domains on the first and second polypeptide chains form one functional binding site for a second antigen, and the VD5 and VD6 domains on the third and fourth polypeptide chains form one functional binding site for a third antigen.

21. The multispecific binding protein of claim 20, wherein the Fc region of the first and third polypeptide chains each comprises a mutation, wherein said mutations on the two Fc regions enhance heterodimerization of the first and third polypeptide chains.

22. The multispecific binding protein of claim 20, wherein the first antigen is the target cell antigen.

23. The multispecific binding protein of claim 20, wherein the second antigen is the TCR complex component.

24. The multispecific binding protein of claim 20, wherein the third antigen is the modulator.

25. The multispecific binding protein of claim 20, wherein VD1, VD2, or VD5 comprises a CDR region amino acid sequence selected from SEQ ID NO: 88-111.

26-27. (canceled)

28. The multispecific binding protein of claim 20, which comprises a polypeptide sequence selected from the group consisting of SEQ ID NO: 32-79.

29. (canceled)

30. A therapeutic combination comprising: a first multispecific binding protein comprising a first binding site that specifically binds to a target cell antigen, and a second binding site that specifically binds to an immune cell receptor complex component on the immune cell; and a second multispecific binding protein comprising a first binding site that specifically binds to a target cell antigen, and a second binding site that specifically binds to a cell surface modulator on the immune cell.

31. The therapeutic combination of claim 30, wherein the target cell antigen is a disease-associated antigen.

32. The therapeutic combination of claim 30, wherein the target cell antigen is tumor-associated antigen.

33-40. (canceled)

41. The therapeutic combination of claim 30, wherein the first and/or second multispecific binding protein is a DVD-Ig, half-DVD-Ig, a scDVD-Ig, an fDVD-Ig, an rDVD-Ig, a pDVD-Ig, an mDVD-Ig or a coDVD-Ig.

42. The therapeutic combination of claim 30, wherein the first multispecific binding protein comprises a polypeptide chain having the formula VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 100, 101, 102; 106, 107, and 108;
(b) VD1 or VD2 comprises HCDR1-3 regions selected from the group consisting of SEQ ID NO: 100, 101, 102; 106, 107, and 108;
(c) VD1 and VD2 comprise HCDR1-3 regions selected from the group consisting of SEQ ID NO: 100, 101, 102; 106, 107, and 108;
(d) VD1 or VD2 comprise a VH amino acid sequence selected from the group consisting of SEQ ID NO: 84 and 86;
(e) VD1 and VD2 comprise SEQ ID NO: 84 and 86, respectively.

43. The therapeutic combination of claim 30, wherein the first multispecific binding protein comprises a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 103, 104, 105, 109, 110, and 111;
(b) VD1 or VD2 comprises LCDR1-3 regions selected from the group consisting of SEQ ID NO: 103, 104, and 105; and 109, 110, and 111;
(c) VD1 and VD2 comprise LCDR1-3 regions selected from the group consisting of SEQ ID NO: 103, 104, and 105; and 109, 110, and 111;
(d) VD1 or VD2 comprise a VL amino acid sequence selected from the group consisting of SEQ ID NO: 85, and 87;
(e) VD1 and VD2 comprise SEQ ID NO: 85 and 87, respectively.

44. The therapeutic combination of claim 30, wherein the second multispecific binding protein comprises a polypeptide chain having the formula VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 88, 89, 90; 94, 95, 96; 100, 101, and 102;
(b) VD1 or VD2 comprises HCDR1-3 regions selected from the group consisting of SEQ ID NO: 88, 89, and 90; 94, 95, and 96; and 100, 101, and 102;
(c) VD1 and VD2 comprise HCDR1-3 regions selected from the group consisting of SEQ ID NO: 88, 89, and 90; 94, 95, and 96; and 100, 101, and 102;
(d) VD1 or VD2 comprise a VH amino acid sequence selected from the group consisting of SEQ ID NO: 80, 82, and 84;
(e) VD1 and VD2 comprise SEQ ID NO: 80 and 84, respectively; or
(f) VD2 and VD2 comprise SEQ ID NO: 82 and 84, respectively.

45. The therapeutic combination of claim 30, wherein the second multispecific binding protein comprises a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 91, 92, 93; 97, 98, 99; 103, 104, and 105;
(b) VD1 or VD2 comprises LCDR1-3 regions selected from the group consisting of SEQ ID NO: 91, 92, and 93; 97, 98, and 99; and 103, 104, and 105;
(c) VD1 and VD2 comprise LCDR1-3 regions selected from the group consisting of SEQ ID NO: 91, 92, and 93; 97, 98, and 99; and 103, 104, and 105;
(d) VD1 or VD2 comprise a VL amino acid sequence selected from the group consisting of SEQ ID NO: 81, 83, and 87;
(e) VD1 and VD2 comprise SEQ ID NO: 81 and 85, respectively; or
(f) VD2 and VD2 comprise SEQ ID NO: 83 and 85, respectively.

46. A method of killing a target cell in a subject comprising administering to the subject an effective amount of the multispecific binding protein of claim 1 or therapeutic combination of claim 45, wherein the therapeutic combination comprises the first multispecific binding protein comprising a first binding site that specifically binds to a target cell antigen, and a second binding site that specifically binds to an immune cell receptor complex component on the immune cell; and a second multispecific binding protein comprising a first binding site that specifically binds to a target cell antigen, and a second binding site that specifically binds to a cell surface modulator on the immune cell.

47. A method of killing a target cell in a subject comprising administering to the subject an effective amount of a multispecific binding protein of any one of claim 1, wherein the multispecific binding protein induces less cytokine production in the subject compared to administration of an equivalent effective amount of a therapeutic combination, wherein the therapeutic combination comprises a first multispecific binding protein comprising a first binding site that specifically binds to a target cell antigen, and a second binding site that specifically binds to an immune cell receptor complex component on the immune cell; and a second multispecific binding protein comprising a first binding site that specifically binds to a target cell antigen, and a second binding site that specifically binds to a cell surface modulator on the immune cell.

48. A method of killing a target cell in a subject comprising administering to the subject an effective amount of a multispecific binding protein of claim 1, wherein the multispecific binding protein induces greater immune cell activation in the subject compared to administration of an equivalent effective amount of a therapeutic combination, wherein the therapeutic combination comprises a first multispecific binding protein comprising a first binding site that specifically binds to a target cell antigen, and a second binding site that specifically binds to an immune cell receptor complex component on the immune cell; and a second multispecific binding protein comprising a first binding site that specifically binds to a target cell antigen, and a second binding site that specifically binds to a cell surface modulator on the immune cell.

49-50. (canceled)

51. A multispecific binding protein comprising: a first binding site that specifically binds to a first T-cell modulator; and a second binding site that specifically binds to a second T-cell modulator.

52-63. (canceled)

64. The multispecific binding protein of claim 51 which is a DVD-Ig, half-DVD-Ig, a scDVD-Ig, an fDVD-Ig, an rDVD-Ig, a pDVD-Ig, an mDVD-Ig or a coDVD-Ig.

65. The multispecific binding protein of claim 51, comprising a polypeptide chain having the formula VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 120, 121, 122, 126, 127, 128, 132, 134, 134, 138, 139, 140;
(b) VD1 or VD2 comprises HCDR1-3 regions selected from the group consisting of SEQ ID NO: 120, 121, and 122; 126, 127, and 128; 132, 134, and 134, and 138, 139, and 140;
(c) VD1 and VD2 comprise HCDR1-3 regions selected from the group consisting of SEQ ID NO: 120, 121, and 122; 126, 127, and 128; 132, 134, and 134, and 138, 139, and 140;
(d) VD1 or VD2 comprise a VH amino acid sequence selected from the group consisting of SEQ ID NO: 112, 114, 115, 116, 118;
(e) VD1 and VD2 comprise a VH amino acid sequence selected from the group consisting of SEQ ID NO: 112, 114, 115, 116, 118; or
(f) the polypeptide chain comprises an amino acid sequence selected from the group consisting of SEQ ID NO: 144, 146, 148, 150, 152, 154, 156, and 158.

66. The multispecific binding protein of claim 51, comprising a polypeptide chain a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein
(a) VD1 or VD2 comprise a CDR region amino acid sequence selected from SEQ ID NO: 123, 124, 125, 129, 130, 131, 135, 136, 137, 141, 142, 143;
(b) VD1 or VD2 comprises HCDR1-3 regions selected from the group consisting of SEQ ID NO: 123, 124, and 125; 129, 130, and 131; 135, 136, and 137; and 141, 142, and 143;
(c) VD1 and VD2 comprise HCDR1-3 regions selected from the group consisting of SEQ ID NO: 123, 124, and 125; 129, 130, and 131; 135, 136, and 137; and 141, 142, and 143;
(d) VD1 or VD2 comprise a VL amino acid sequence selected from the group consisting of SEQ ID NO: 113, 115, 117, and 119;
(e) VD1 and VD2 comprise a VL amino acid sequence selected from the group consisting of SEQ ID NO: 113, 115, 117, and 119; or
(f) the polypeptide chain comprises an amino acid sequence selected from the group consisting of SEQ ID NO: 145, 147, 149, 151, 153, 155, 157, and 159.

67. A method of modulating the activation state of an immune cell, the method comprising contacting the immune cell with a multispecific binding protein of claim 51.

68-69. (canceled)

70. A method of directing a cytotoxic T-cell to lyse a target cell in a subject, the method comprising administering to the subject an effective amount of a multispecific binding protein of claim 51.

71. (canceled)

72. A method of reducing inflammation in a subject, the method comprising administering to the subject an effective amount of a multispecific binding protein of claim 51.

73. (canceled)

74. A multispecific binding protein of claim 1, wherein the binding protein is a crystallized binding protein.

75. An isolated nucleic acid encoding the binding protein amino acid sequence of claim 1.

76. A vector comprising an isolated nucleic acid of claim 75.

77. A host cell comprising a vector of claim 76.

78. A method of producing a multispecific binding protein, comprising culturing the host cell described in claim 77 in culture medium under conditions sufficient to produce the binding protein.

79. A pharmaceutical composition comprising the binding protein of claim 1, and a pharmaceutically acceptable carrier.

80. A method of identifying a multispecific binding protein that modulates a T-cell response, the method comprising:
a) contacting a population of cytotoxic T-cells with a population of target cells in the presence and absence of the multispecific binding protein of claim 1, wherein the multispecific binding protein simultaneously and specifically binds to a cell surface co-stimulator or co-repressor on T-cells and to a cell surface antigen on a target cell; and
b) measuring the amount cell killing of the population of target cells, wherein an increase or decrease in cell killing in the presence of the multispecific binding, compared to the absence of the multispecific binding protein, identifies the multispecific binding protein as modulator of a T-cell response.

81. A method of identifying a multispecific binding protein that modulates a T-cell response, the method comprising:
a) contacting a population of cytotoxic T-cells with a population of antigen presenting cells in the presence and absence of the multispecific binding protein of claim 1, wherein the multispecific binding protein simultaneously and specifically binds to two cell surface co-stimulators or co-repressors on T-cells; and
b) measuring the amount of cytokine released from the population of T-cells, wherein an increase or decrease in amount of cytokine released in the presence of the multispecific binding, compared to the absence of the multispecific binding protein, identifies the multispecific binding protein as modulator of a T-cell response.

82. A method of modulating an immune response in a subject, the method comprising: administering a therapeutic amount of the multispecific binding protein of claim 1 that simultaneously and specifically binds to a cell surface co-stimulator or co-repressor on an T-cell and to a cell surface antigen on a target cell, wherein the multispecific binding protein modulates a normal response of the T-cell to target cell binding.


SEQUENCE LISTING (159 sequences)

SEQ ID NO: 1 - protein, 16 aa, Artificial Sequence
  Description of Artificial Sequence Synthetic peptide
AKTTPKLEEGEFSEAR

SEQ ID NO: 2 - protein, 17 aa, Artificial Sequence
  Description of Artificial Sequence Synthetic peptide
AKTTPKLEEGEFSEARV

SEQ ID NO: 3 - protein, 9 aa, Artificial Sequence
  Description of Artificial Sequence Synthetic peptide
AKTTPKLGG

SEQ ID NO: 4 - protein, 10 aa, Artificial Sequence
  Description of Artificial Sequence Synthetic peptide
SAKTTPKLGG

SEQ ID NO: 5 - protein, 6 aa, Artificial Sequence
  Description of Artificial Sequence Synthetic peptide
SAKTTP

SEQ ID NO: 6 - protein, 6 aa, Artificial Sequence
  Description of Artificial Sequence Synthetic peptide
RADAAP

SEQ ID NO: 7 - protein, 9 aa, Artificial Sequence
  Description of Artificial Sequence Synthetic peptide
RADAAPTVS

SEQ ID NO: 8 - protein, 12 aa, Artificial Sequence
  Description of Artificial Sequence Synthetic peptide
RADAAAAGGPGS

SEQ ID NO: 9 - protein, 27 aa, Artificial Sequence
  Description of Artificial Sequence Synthetic peptide
RADAAAAGGGGSGGGGSGGGGSGGGGS

SEQ ID NO: 10 - protein, 18 aa, Artificial Sequence
  Description of Artificial Sequence Synthetic peptide
SAKTTPKLEEGEFSEARV

SEQ ID NO: 11 - protein, 5 aa, Artificial Sequence
  Description of Artificial Sequence Synthetic peptide
ADAAP

SEQ ID NO: 12 - protein, 12 aa, Artificial Sequence
  Description of Artificial Sequence Synthetic peptide
ADAAPTVSIFPP

SEQ ID NO: 13 - protein, 5 aa, Artificial Sequence
  Description of Artificial Sequence Synthetic peptide
TVAAP

SEQ ID NO: 14 - protein, 12 aa, Artificial Sequence
  Description of Artificial Sequence Synthetic peptide
TVAAPSVFIFPP

SEQ ID NO: 15 - protein, 6 aa, Artificial Sequence
  Description of Artificial Sequence Synthetic peptide
QPKAAP

SEQ ID NO: 16 - protein, 13 aa, Artificial Sequence
  Description of Artificial Sequence Synthetic peptide
QPKAAPSVTLFPP

SEQ ID NO: 17 - protein, 6 aa, Artificial Sequence
  Description of Artificial Sequence Synthetic peptide
AKTTPP

SEQ ID NO: 18 - protein, 13 aa, Artificial Sequence
  Description of Artificial Sequence Synthetic peptide
AKTTPPSVTPLAP

SEQ ID NO: 19 - protein, 6 aa, Artificial Sequence
  Description of Artificial Sequence Synthetic peptide
AKTTAP

SEQ ID NO: 20 - protein, 13 aa, Artificial Sequence
  Description of Artificial Sequence Synthetic peptide
AKTTAPSVYPLAP

SEQ ID NO: 21 - protein, 6 aa, Artificial Sequence
  Description of Artificial Sequence Synthetic peptide
ASTKGP

SEQ ID NO: 22 - protein, 13 aa, Artificial Sequence
  Description of Artificial Sequence Synthetic peptide
ASTKGPSVFPLAP

SEQ ID NO: 23 - protein, 15 aa, Artificial Sequence
  Description of Artificial Sequence Synthetic peptide
GGGGSGGGGSGGGGS

SEQ ID NO: 24 - protein, 15 aa, Artificial Sequence
  Description of Artificial Sequence Synthetic peptide
GENKVEYAPALMALS

SEQ ID NO: 25 - protein, 15 aa, Artificial Sequence
  Description of Artificial Sequence Synthetic peptide
GPAKELTPLKEAKVS

SEQ ID NO: 26 - protein, 15 aa, Artificial Sequence
  Description of Artificial Sequence Synthetic peptide
GHEAAAVMQVQYPAS

SEQ ID NO: 27 - protein, 24 aa, Artificial Sequence
  Description of Artificial Sequence Synthetic peptide
TVAAPSVFIFPPTVAAPSVFIFPP

SEQ ID NO: 28 - protein, 26 aa, Artificial Sequence
  Description of Artificial Sequence Synthetic peptide
ASTKGPSVFPLAPASTKGPSVFPLAP

SEQ ID NO: 29 - protein, 5 aa, Artificial Sequence
  Description of Artificial Sequence Synthetic peptide
GGGGS

SEQ ID NO: 30 - protein, 10 aa, Artificial Sequence
  Description of Artificial Sequence Synthetic peptide
GGGGSGGGGS

SEQ ID NO: 31 - protein, 9 aa, Artificial Sequence
  Description of Artificial Sequence Synthetic peptide
GGSGGGGSG

SEQ ID NO: 32 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 585 aa, Artificial Sequence
  Description of Artificial Sequence Synthetic polypeptide
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 323 aa, Artificial Sequence
  Description of Artificial Sequence Synthetic polypeptide
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRGGSGGGGSGQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 36 - protein, 214 aa, Artificial Sequence; identical to SEQ ID NO: 32
  Description of Artificial Sequence Synthetic polypeptide

SEQ ID NO: 37 - protein, 449 aa, Artificial Sequence; identical to SEQ ID NO: 33
  Description of Artificial Sequence Synthetic polypeptide

SEQ ID NO: 38 - protein, 585 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSEVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39 - protein, 323 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRGGSGGGGSGDIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 40 - protein, 214 aa, Artificial Sequence; identical to SEQ ID NO: 32
  Description of Artificial Sequence Synthetic polypeptide

SEQ ID NO: 41 - protein, 449 aa, Artificial Sequence; identical to SEQ ID NO: 33
  Description of Artificial Sequence Synthetic polypeptide

SEQ ID NO: 42 - protein, 585 aa, Artificial Sequence; identical to SEQ ID NO: 38
  Description of Artificial Sequence Synthetic polypeptide

SEQ ID NO: 43 - protein, 323 aa, Artificial Sequence; identical to SEQ ID NO: 39
  Description of Artificial Sequence Synthetic polypeptide

SEQ ID NO: 44 - protein, 214 aa, Artificial Sequence; identical to SEQ ID NO: 32
  Description of Artificial Sequence Synthetic polypeptide

SEQ ID NO: 45 - protein, 449 aa, Artificial Sequence; identical to SEQ ID NO: 33
  Description of Artificial Sequence Synthetic polypeptide

SEQ ID NO: 46 - protein, 591 aa, Artificial Sequence
  Description of Artificial Sequence Synthetic polypeptide
EVQLVESGGGLVQPGGSLRLSCAASGFTFSTYAMNWVRQAPGKGLEWVGRIRSKYNNYATYYADSVKDRFTISRDDSKNTAYLQMNSLRAEDTAVYYCTRHGNFGNSYVSWFAYWGQGTLVTVSSGGGGSGGGGSEVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 327 aa, Artificial Sequence
  Description of Artificial Sequence Synthetic polypeptide
DAQVTQSPSSLSASVGDRVTITCRSSTGAVTTSNYANWVQEKPGKLFKGLIGGTNKRAPGVPSRFSGSGSGTDATLTISSLQPEDFATYFCALWYSNLWVFGGGTKVEIKRGGSGGGGSGDIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 48 - protein, 214 aa, Artificial Sequence
  Description of Artificial Sequence Synthetic polypeptide
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 393 aa, Artificial Sequence
  Description of Artificial Sequence Synthetic polypeptide
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKT

SEQ ID NO: 50 - protein, 584 aa, Artificial Sequence
  Description of Artificial Sequence Synthetic polypeptide
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 323 aa, Artificial Sequence
  Description of Artificial Sequence Synthetic polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRGGSGGGGSGQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 52 - protein, 214 aa, Artificial Sequence; identical to SEQ ID NO: 48
  Description of Artificial Sequence Synthetic polypeptide

SEQ ID NO: 53 - protein, 393 aa, Artificial Sequence; identical to SEQ ID NO: 49
  Description of Artificial Sequence Synthetic polypeptide

SEQ ID NO: 54 - protein, 590 aa, Artificial Sequence
  Description of Artificial Sequence Synthetic polypeptide
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSGGGGSGGGGSEVQLVESGGGLVQPGGSLRLSCAASGFTFSTYAMNWVRQAPGKGLEWVGRIRSKYNNYATYYADSVKDRFTISRDDSKNTAYLQMNSLRAEDTAVYYCTRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 327 aa, Artificial Sequence
  Description of Artificial Sequence Synthetic polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRGGSGGGGSGDAQVTQSPSSLSASVGDRVTITCRSSTGAVTTSNYANWVQEKPGKLFKGLIGGTNKRAPGVPSRFSGSGSGTDATLTISSLQPEDFATYFCALWYSNLWVFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 56 - protein, 214 aa, Artificial Sequence; identical to SEQ ID NO: 48
  Description of Artificial Sequence Synthetic polypeptide

SEQ ID NO: 57 - protein, 393 aa, Artificial Sequence; identical to SEQ ID NO: 49
  Description of Artificial Sequence Synthetic polypeptide

SEQ ID NO: 58 - protein, 587 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 323 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGSGGGGSGQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 60 - protein, 216 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 451 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 584 aa, Artificial Sequence; identical to SEQ ID NO: 50
  Description of Artificial Sequence Synthetic polypeptide

SEQ ID NO: 63 - protein, 323 aa, Artificial Sequence; identical to SEQ ID NO: 51
  Description of Artificial Sequence Synthetic polypeptide

SEQ ID NO: 64 - protein, 216 aa, Artificial Sequence; identical to SEQ ID NO: 60
  Description of Artificial Sequence Synthetic polypeptide

SEQ ID NO: 65 - protein, 451 aa, Artificial Sequence; identical to SEQ ID NO: 61
  Description of Artificial Sequence Synthetic polypeptide

SEQ ID NO: 66 - protein, 590 aa, Artificial Sequence; identical to SEQ ID NO: 54
  Description of Artificial Sequence Synthetic polypeptide

SEQ ID NO: 67 - protein, 327 aa, Artificial Sequence; identical to SEQ ID NO: 55
  Description of Artificial Sequence Synthetic polypeptide

SEQ ID NO: 68 - protein, 216 aa, Artificial Sequence; identical to SEQ ID NO: 60
  Description of Artificial Sequence Synthetic polypeptide

SEQ ID NO: 69 - protein, 451 aa, Artificial Sequence; identical to SEQ ID NO: 61
  Description of Artificial Sequence Synthetic polypeptide

SEQ ID NO: 70 - protein, 587 aa, Artificial Sequence; identical to SEQ ID NO: 58
  Description of Artificial Sequence Synthetic polypeptide

SEQ ID NO: 71 - protein, 323 aa, Artificial Sequence; identical to SEQ ID NO: 59
  Description of Artificial Sequence Synthetic polypeptide

SEQ ID NO: 72 - protein, 214 aa, Artificial Sequence; identical to SEQ ID NO: 48
  Description of Artificial Sequence Synthetic polypeptide

SEQ ID NO: 73 - protein, 447 aa, Artificial Sequence
  Description of Artificial Sequence Synthetic polypeptide
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 581 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 333 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 216 aa, Artificial Sequence; identical to SEQ ID NO: 60
  Description of Artificial Sequence Synthetic polypeptide

SEQ ID NO: 77 - protein, 451 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 581 aa, Artificial Sequence; identical to SEQ ID NO: 74
  Description of Artificial Sequence Synthetic polypeptide

SEQ ID NO: 79 - protein, 333 aa, Artificial Sequence; identical to SEQ ID NO: 75
  Description of Artificial Sequence Synthetic polypeptide

SEQ ID NO: 80 - protein, 447 aa, Artificial Sequence
  Description of Artificial Sequence Synthetic polypeptide
EVQLVESGGGLVMPGGSLKLSCAASGFAFXSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, Artificial Sequence; identical to SEQ ID NO: 48
  Description of Artificial Sequence Synthetic polypeptide

SEQ ID NO: 82 - protein, 451 aa, Artificial Sequence; identical to SEQ ID NO: 61
  Description of Artificial Sequence Synthetic polypeptide

SEQ ID NO: 83 - protein, 216 aa, Artificial Sequence; identical to SEQ ID NO: 60
  Description of Artificial Sequence Synthetic polypeptide

SEQ ID NO: 84 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 214 aa, Artificial Sequence; identical to SEQ ID NO: 32
  Description of Artificial Sequence Synthetic polypeptide

SEQ ID NO: 86 - protein, 119 aa, Artificial Sequence [sequence omitted]

SEQ ID NO: 87 - protein, 107 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINR

SEQ ID NO: 88 - protein, 5 aa, Artificial Sequence
  Description of Artificial Sequence Synthetic peptide
SYDMS

SEQ ID NO: 89 - protein, 16 aa, Artificial Sequence
  Description of Artificial Sequence Synthetic peptide
YISGGGFTYYPDTVKG

SEQ ID NO: 90 - protein, 9 aa, Artificial Sequence
  Description of Artificial Sequence Synthetic peptide
QGANWELVY

SEQ ID NO: 91 - protein, 11 aa, Artificial Sequence
  Description of Artificial Sequence Synthetic peptide
RASQSISDFLH

SEQ ID NO: 92 - protein, 7 aa, Artificial Sequence
  Description of Artificial Sequence Synthetic peptide
YASQSIS

SEQ ID NO: 93 - protein, 9 aa, Artificial Sequence
  Description of Artificial Sequence Synthetic peptide
QNGHNFPPT

SEQ ID NO: 94 - protein, 5 aa, Artificial Sequence
  Description of Artificial Sequence Synthetic peptide
GYYWS

SEQ ID NO: 95 - protein, 16 aa, Artificial Sequence
  Description of Artificial Sequence Synthetic peptide
EINHGGYVTYNPSLES

SEQ ID NO: 96 - protein, 13 aa, Artificial Sequence
  Description of Artificial Sequence Synthetic peptide
DYGPGNYDWYFDL

SEQ ID NO: 97 - protein, 11 aa, Artificial Sequence
  Description of Artificial Sequence Synthetic peptide
RASQSVSSYLA

SEQ ID NO: 98 - protein, 7 aa, Artificial Sequence
  Description of Artificial Sequence Synthetic peptide
DASNRAT

SEQ ID NO: 99 - protein, 11 aa, Artificial Sequence
  Description of Artificial Sequence Synthetic peptide
QQRSNWPPALT

SEQ ID NO: 100 - protein, 5 aa, Artificial Sequence
  Description of Artificial Sequence Synthetic peptide
NYGVH

SEQ ID NO: 101 - protein, 16 aa, Artificial Sequence
  Description of Artificial Sequence Synthetic peptide
VIWSGGNTDYNTPFTS

SEQ ID NO: 102 - protein, 11 aa, Artificial Sequence
  Description of Artificial Sequence Synthetic peptide
ALTYYDYEFAY

SEQ ID NO: 103 - protein, 11 aa, Artificial Sequence
  Description of Artificial Sequence Synthetic peptide
RASQSIGTNIH

SEQ ID NO: 104 - protein, 7 aa, Artificial Sequence
  Description of Artificial Sequence Synthetic peptide
YASESIS

SEQ ID NO: 105 - protein, 9 aa, Artificial Sequence
  Description of Artificial Sequence Synthetic peptide
QQNNNWPTT

SEQ ID NO: 106 - protein, 5 aa, Artificial Sequence
  Description of Artificial Sequence Synthetic peptide
RYTMH

SEQ ID NO: 107 - protein, 17 aa, Artificial Sequence
  Description of Artificial Sequence Synthetic peptide
YINPSRGYTNYNQKFKD

SEQ ID NO: 108 - protein, 10 aa, Artificial Sequence
  Description of Artificial Sequence Synthetic peptide
YYDDHYCLDY

SEQ ID NO: 109 - protein, 10 aa, Artificial Sequence
  Description of Artificial Sequence Synthetic peptide
RASSSVSYMN

SEQ ID NO: 110 - protein, 7 aa, Artificial Sequence
  Description of Artificial Sequence Synthetic peptide
DTSKVAS

SEQ ID NO: 111 - protein, 9 aa, Artificial Sequence
  Description of Artificial Sequence Synthetic peptide
QQWSSNPLT

SEQ ID NO: 112 - protein, 113 aa, Artificial Sequence [sequence omitted]

SEQ ID NO: 113 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 114 - protein, 118 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 115 - protein, 109 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 116 - protein, 120 aa, Artificial Sequence
  Description of Artificial Sequence Synthetic polypeptide
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 117 - protein, 108 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKR

SEQ ID NO: 118 - protein, 121 aa, Artificial Sequence [sequence omitted]

SEQ ID NO: 119 - protein, 110 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKR

SEQ ID NO: 120 - protein, 10 aa, Artificial Sequence
  Description of Artificial Sequence Synthetic peptide
GITFSNSGMH

SEQ ID NO: 121 - protein, 17 aa, Artificial Sequence
  Description of Artificial Sequence Synthetic peptide
VIWYDGSKRYYADSVKG

SEQ ID NO: 122 - protein, 4 aa, Artificial Sequence
  Description of Artificial Sequence Synthetic peptide
NDDY

SEQ ID NO: 123 - protein, 11 aa, Artificial Sequence
  Description of Artificial Sequence Synthetic peptide
RASQSVSSYLA

SEQ ID NO: 124 - protein, 7 aa, Artificial Sequence
  Description of Artificial Sequence Synthetic peptide
YDASNRA

SEQ ID NO: 125 - protein, 9 aa, Artificial Sequence
  Description of Artificial Sequence Synthetic peptide
QQSSNWPRT

SEQ ID NO: 126 - protein, 10 aa, Artificial Sequence
  Description of Artificial Sequence Synthetic peptide
GFTFSSYTMH

SEQ ID NO: 127 - protein, 17 aa, Artificial Sequence
  Description of Artificial Sequence Synthetic peptide
FISYDGNNKYYADSVKG

SEQ ID NO: 128 - protein, 9 aa, Artificial Sequence
  Description of Artificial Sequence Synthetic peptide
TGWLGPFDY

SEQ ID NO: 129 - protein, 9 aa, Artificial Sequence
  Description of Artificial Sequence Synthetic peptide
QSVGSSYLA

SEQ ID NO: 130 - protein, 7 aa, Artificial Sequence
  Description of Artificial Sequence Synthetic peptide
YGAFSRA

SEQ ID NO: 131 - protein, 9 aa, Artificial Sequence
  Description of Artificial Sequence Synthetic peptide
QQYGSSPWT

SEQ ID NO: 132 - protein, 10 aa, Artificial Sequence
  Description of Artificial Sequence Synthetic peptide
GGSFSDYYWN

SEQ ID NO: 133 - protein, 16 aa, Artificial Sequence
  Description of Artificial Sequence Synthetic peptide
EINHNGNTNSNPSLKS

SEQ ID NO: 134 - protein, 12 aa, Artificial Sequence
  Description of Artificial Sequence Synthetic peptide
GYSDYEYNWFDP

SEQ ID NO: 135 - protein, 8 aa, Artificial Sequence
  Description of Artificial Sequence Synthetic peptide
QSISSYLA

SEQ ID NO: 136 - protein, 7 aa, Artificial Sequence
  Description of Artificial Sequence Synthetic peptide
YDASNRA

SEQ ID NO: 137 - protein, 9 aa, Artificial Sequence
  Description of Artificial Sequence Synthetic peptide
QQRSNWPLT

SEQ ID NO: 138 - protein, 10 aa, Artificial Sequence
  Description of Artificial Sequence Synthetic peptide
GGSFSGYYWS

SEQ ID NO: 139 - protein, 16 aa, Artificial Sequence
  Description of Artificial Sequence Synthetic peptide
EINHGGYVTYNPSLES

SEQ ID NO: 140 - protein, 13 aa, Artificial Sequence
  Description of Artificial Sequence Synthetic peptide
DYGPGNYDWYFDL

SEQ ID NO: 141 - protein, 8 aa, Artificial Sequence
  Description of Artificial Sequence Synthetic peptide
QSVSSYLA

SEQ ID NO: 142 - protein, 7 aa, Artificial Sequence
  Description of Artificial Sequence Synthetic peptide
YDASNRA

SEQ ID NO: 143 - protein, 11 aa, Artificial Sequence
  Description of Artificial Sequence Synthetic peptide
QQRSNWPPALT

SEQ ID NO: 144 - protein, 239 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 145 - protein, 221 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPEIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKR

SEQ ID NO: 146 - protein, 246 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 147 - protein, 221 aa, Artificial Sequence; identical to SEQ ID NO: 145
  Description of Artificial Sequence Synthetic polypeptide

SEQ ID NO: 148 - protein, 239 aa, Artificial Sequence
  Description of Artificial Sequence Synthetic polypeptide
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSASTKGPQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 149 - protein, 221 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 150 - protein, 246 aa, Artificial Sequence
  Description of Artificial Sequence Synthetic polypeptide
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSASTKGPSVFPLAPQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 151 - protein, 221 aa, Artificial Sequence; identical to SEQ ID NO: 149
  Description of Artificial Sequence Synthetic polypeptide

SEQ ID NO: 152 - protein, 247 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 153 - protein, 223 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPEIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKR

SEQ ID NO: 154 - protein, 254 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPQVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 155 - protein, 223 aa, Artificial Sequence; identical to SEQ ID NO: 153
  Description of Artificial Sequence Synthetic polypeptide

SEQ ID NO: 156 - protein, 247 aa, Artificial Sequence
  Description of Artificial Sequence Synthetic polypeptide
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSASTKGPQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSS

SEQ ID NO: 157 - protein, 223 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKR

SEQ ID NO: 158 - protein, 254 aa, Artificial Sequence
  Description of Artificial Sequence Synthetic polypeptide
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSSASTKGPSVFPLAPQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSS

SEQ ID NO: 159 - protein, 223 aa, Artificial Sequence; identical to SEQ ID NO: 157
  Description of Artificial Sequence Synthetic polypeptide